(19) United States
(12) Patent Application Publication
(10) Pub. No.: US 2023/0091336 A1
(43) Pub. Date: Mar. 23, 2023

(54) CANNABINOID COMPOSITIONS

(71) Applicant: The Queen's Medical Center, Honolulu, HI (US)

(72) Inventors: Reinhold PENNER, Honolulu, HI (US); Ram P. NEUPANE, Aiea, HI (US); Sayuri SUZUKI, Honolulu, HI (US); Malika FAOUZI, Honolulu, HI (US)

(21) Appl. No.: 17/904,934

(22) PCT Filed: Feb. 24, 2021
(86) PCT No.: PCT/US2021/019474

§ 371 Date: Aug. 24, 2022

Related U.S. Application Data
(60) Provisional application No. 62/981,424, filed on Feb. 25, 2020.
(60) Provisional application No. 63/072,806, filed on Aug. 31, 2020.

(51) Int. Cl. A61K 31/192; A61K 31/05; A61K 31/352

(52) U.S. Cl. CPC A61K 31/192; A61K 31/05; A61K 31/352

(57) ABSTRACT

Described herein are pharmaceutical compositions comprising cannabinoids. In some embodiments, such compositions are useful for the treatment of inflammatory or autoimmnune diseases or disorders. Further provided herein are pharmaceutical compositions comprising combinations of cannabinoids possessing entourage effects.

### CROSS-REFERENCE

This patent application claims the benefit of U.S. Provisional Patent Application No. 62/981,424, filed on Feb. 25, 2020; and U.S. Provisional Patent Application No. 63/072,806, filed on Aug. 31, 2020; each of which is incorporated herein by reference in its entirety.

### INCORPORATION BY REFERENCE

All publications, patents, and patent applications mentioned, disclosed or referenced in this specification are herein incorporated by reference in their entirety and to the same extent as if each individual publication, patent, or patent application was specifically and individually indicated to be incorporated by reference.

### BACKGROUND

Chronic pain and inflammation—often arising from musculoskeletal injury, nervous system dysfunction, chronic diseases, cancer, and autoimmune disorders—affects millions of individuals across the globe. Opioid analgesics are commonly prescribed in some countries because they are effective in acutely relieving many types of pain, however, their long-term use carries the risk for addiction and abuse. Overreliance on opioid analgesics has resulted in a public health crisis in many jurisdictions where the population has access to opioids. There is an urgent need to identify and develop alternative pain management strategies for treating chronic pain and the underlying diseases causing pain.

### SUMMARY OF THE INVENTION

Described herein are compositions, methods, and systems comprising cannabinoids. In some embodiments, disclosed herein are compositions, methods and systems for compositions comprising cannabigerolic acid (CBGA) and a second cannabinoid compound. In some embodiments, disclosed herein are compositions, methods and systems for compositions comprising cannabigerolic acid (CBGA) and a second cannabinoid compound, wherein the CBGA is present in an amount from 1 mg to 2500 mg, and the second cannabinoid compound is present in an amount from 1 mg to 2500 mg. In some embodiments, the compositions are pharmaceutical. In other embodiments, the CBGA is present in an amount from 5 mg to 1200 mg. In yet other instances, the second cannabinoid compound is present in an amount from 5 mg to 1200 mg. In some embodiments, the compositions inhibit secretion of inflammatory cytokines from at least one immune cell. In other instances, the at least one immune cell type is a lymphocyte. In still other instances, the at least one immune cell type is a monocyte or a macrophage. In yet other instances, the at least one immune cell type is a microglia cell. In some embodiments, the composition inhibits secretion of inflammatory cytokines by at least two immune cell types. In some embodiments, at least one immune cell type is a lymphocyte and at least one immune cell type is a monocyte or a macrophage. In other embodiments, at least one immune cell type is a lymphocyte and at least one immune cell type is a mast cell. In other embodiments, the second cannabinoid compound and the CBGA disclosed herein have an additive effect as measured by combination indices (CI) according to the method of isoboles. In other embodiments, the second cannabinoid and the CBGA have a supra-additive effect as measured by combination indices (CI) according to the method of isoboles. In still other embodiments, the second cannabinoid and the CBGA have a sub-additive effect as measured by combination indices (CI) according to the method of isoboles. In some embodiments, the second cannabinoid is cannabidiolic acid (CBDA). In other instances, the second cannabinoid is cannabidivarin (CBDV). In still other embodiments, the second cannabinoid is cannabigerol (CBG). In yet other instances, the second cannabinoid is cannabidiol (CBD). In still other instances, the second cannabinoid is tetrahydrocannabinolic acid (THCA). In yet other embodiments, the second cannabinoid is cannabigerovarinic acid (CBGVA). In some instances, the second cannabinoid is tetrahydrocannabivarinic acid (THCVA). In other instances, the compositions disclosed herein comprise a starting material for the CBGA derived from a plant. In still other instances, temperatures below 45° C. are used to extract the CBGA from the plant. In some instances, the CBGA is synthetic. In yet other embodiments, the CBGA is recombinantly expressed. In still other embodiments, a starting material for the second cannabinoid is plant based. In yet other instances, a starting material for the second cannabinoid is synthetic. In still other instances, a starting material for the second cannabinoid is recombinantly expressed. In some instances, the composition is in a unit dose form. In other instances, the unit dose form is packaged into a container selected from the group consisting of a tube, a jar, a vial, a bag, a tray, a drum, a bottle, a syringe, a vape cartridge, and a can. In still other instances, the container contains information describing directions for use in a subject. In yet other instances, the subject is a human.

Also disclosed herein are compositions, systems and methods of treating a pain or inflammation in a subject in need thereof, comprising administering to the subject a pharmaceutical composition comprising cannabigerolic acid (CBGA) and a second cannabinoid compound, wherein the CBGA is present in an amount from 1 mg to 2500 mg, and the second cannabinoid compound is present in an amount from 1 mg to 2500 mg. In some instances, the CBGA is present in an amount from 5 mg to 1200 mg. In other instances, the second cannabinoid compound is present in an amount from 5 mg to 1200 mg. In some instances, the pharmaceutical composition inhibits secretion of inflammatory cytokines from at least one immune cell. In other instances, the at least one immune cell type is a lymphocyte. In still other instances, the at least one immune cell type is a monocyte or a macrophage. In yet other instances, the at least one immune cell type is a microglia. In some instances, the pharmaceutical composition inhibits secretion of inflammatory cytokines by at least two immune cell types. In some instances, at least one immune cell type is a lymphocyte and at least immune cell type is a monocyte or a macrophage. In other instances, at least one immune cell type is a lymphocyte and at least immune cell type is a mast cell. In some embodiments, the second cannabinoid and the CBGA have an additive effect as measured by combination indices (CI) according to the method of isoboles. In yet other instances, the second cannabinoid and the CBGA have a supra-additive effect as measured by combination indices (CI) according to the method of isoboles. In still other instances, the second cannabinoid and the CBGA have a sub-additive effect as measured by combination indices (CI) according to the method of isoboles. In some embodiments, the second cannabinoid is cannabidiolic acid (CBDA). In other instances, the second cannabinoid is cannabidivarin (CBDV). In still other instances, the second cannabinoid is cannabigerol (CBG). In still other instances, the second cannabinoid is cannabidiol (CBD). In yet other instances, the second cannabinoid is tetrahydrocannabinolic acid (THCA). In yet other instances, the second cannabinoid is cannabigerovarinic acid (CBGVA). In still other instances, the second cannabinoid is tetrahydrocannabivarinic acid (THCVA). In some instances, a starting material for the CBGA and/or second cannabinoid is plant based. In other instances, the CBGA and/or second cannabinoid is synthetic. In yet other instances, the CBGA and/or second cannabinoid is recombinantly expressed.

Also disclosed herein are systems, methods and pharmaceutical compositions comprising a therapeutically-effective amount of cannabigerolic acid (CBGA), wherein the pharmaceutical composition has no more than 2500 mg of cannabidivarin (CBDV), wherein the pharmaceutical composition is formulated for administration to a subject. In some instances, the therapeutically-effective amount of CBGA is at least 1 mg. In some instances, the pharmaceutical composition has no more than 1200 mg of CBDV. In yet other instances, the pharmaceutical composition is substantially free of CBDV. In yet other instances, the cannabigerolic acid (CBGA) is substantially pure. In some embodiments, the pharmaceutical compositions disclosed herein further comprise an amount of cannabidiolic acid (CBDA). In some embodiments, the pharmaceutical compositions further comprise an amount of tetrahydrocannabinolic acid (THCA). In yet other instances, the pharmaceutical compositions further comprise an amount of cannabigerol (CBG). In still other embodiments, the pharmaceutical composition furthers comprise an amount of cannabidiol (CBD). In still other instances, the pharmaceutical compositions do not comprise delta-9-tetrahydrocannabinol (Δ9-THC).

Also disclosed herein are compositions, systems and methods of treating a pain or inflammation in a subject in need thereof, comprising administering to the subject a pharmaceutical composition comprising an amount of cannabigerolic acid (CBGA), and comprising no more than 1 mg of a second cannabinoid, wherein the second cannabinoid is CBG, CBD, DBCV, THC, THCA and CBDA, wherein the CBGA suppresses a pro-inflammatory activity of an immune cell. In some instances, the CBGA suppresses the pro-inflammatory activity of the immune cell by inhibiting immune cell activation. In other instances, the CBGA inhibits a Ca²⁺ influx mechanisms present in an immune cell of the subject. In still other embodiments, the Ca²⁺ influx mechanism is Store-Operated Calcium Entry. In yet other instances, the pharmaceutical compositions further comprise an amount of cannabidiolic acid (CBDA), wherein the CBDA and the CBGA have a sub-additive effect in suppressing the pro-inflammatory activity of the immune cell. In some instances, the pharmaceutical composition further comprises an amount of tetrahydrocannabinolic acid (THCA), wherein the THCA and the CBGA have an additive effect in suppressing the pro-inflammatory activity of the immune cell. In yet other instances, the pharmaceutical compositions further comprise an amount of cannabigerol (CBG), wherein the CBG and the CBGA have a supra-additive effect in suppressing the pro-inflammatory activity of the immune cell. In still other embodiments, the pharmaceutical compositions further comprise an amount of cannabiodiol (CBD), wherein the CBD and the CBGA have a supra-additive effect in suppressing the pro-inflammatory activity of the immune cell. In still other embodiments, the pharmaceutical compositions further comprise an amount of cannabidivarin (CBDV), wherein the CBD and the CBDV have a supra-additive effect in suppressing the pro-inflammatory activity of the immune cell. In some embodiments, the immune cell is a mast cell, a neutrophil, a monocyte, a macrophage, or a lymphocyte. In other instances, the pain is selected from the group consisting of chronic pain, acute, nociceptive, breakthrough, soft tissue, visceral, somatic, phantom, cancer, inflammatory, and neuropathic pain. In yet other instances, the pain is chronic neuropathic pain.

Also disclosed herein are methods, systems and compositions for treating fibrosis in a subject in need thereof, comprising administering to the subject a pharmaceutical composition comprising a therapeutically effective amount of cannabigerolic acid (CBGA). In some instances, the therapeutically effective amount of CBGA is between 0.1-50 mg/kg. In other instances, CBGA inhibits TRPM7 activity. In yet other instances, the pharmaceutical composition further comprises an amount of cannabidiolic acid (CBDA), wherein the CBDA and the CBGA have a sub-additive effect in treating fibrosis. In still other instances, the pharmaceutical composition further comprises an amount of tetrahydrocannabinolic acid (THCA), wherein the THCA and the CBGA have an additive effect in treating fibrosis. In some instances, the pharmaceutical composition further comprises an amount of cannabigerol (CBG), wherein the CBG and the CBGA have a supra-additive effect in treating fibrosis. In still other instances, the pharmaceutical composition further comprises an amount of cannabiodiol (CBD), wherein the CBD and the CBGA have a supra-additive effect in treating fibrosis. In yet other instances, the pharmaceutical composition further comprises an amount of cannabidivarin (CBDV), wherein the CBD and the CBDV have a supra-additive effect in treating fibrosis. In some instances, the fibrosis is renal fibrosis. In other instances, the renal fibrosis is associated with Chronic Kidney Disease (CKD).

Also disclosed herein are methods, systems and compositions comprising (i) no more than 80% by weight of cannabidiolic acid (CBDA); (ii) additives at a concentration of at most 5% by weight, wherein the additives are selected from the group consisting of pharmaceutically acceptable excipients, carriers, diluents, solubilizers, flavorants, colorants, and adjuvants; and (iii) impurities at a concentration of at most 15% by weight, wherein impurities may be cannabinoid compounds, terpenoid compounds, water, solvents, or salts, as measured by high performance liquid chromatography (HPLC). In some embodiments, the compositions further comprise a methyl analog of a cannabinoid. In yet other embodiments, the compositions further comprise a dimethyl analog of a cannabinoid. In some embodiments, the composition comprises no more than 80%, no more than 75%, no more than 70%, or no more than 65% by weight of cannabidiolic acid (CBDA) and cannabigerolic acid (CBGA). In yet other embodiments, the composition comprises no more than 80%, no more than 75%, no more than 70%, or no more than 65% by weight of cannabidiolic acid (CBDA), cannabigerolic acid (CBGA), and cannabigerol (CBG). In still other embodiments, the composition comprises no more than 80% no more than 75%, no more than 70%, or no more than 65% by weight of cannabidiolic acid (CBDA), cannabigerolic acid (CBGA), cannabigerol (CBG), and cannabiodiol (CBD). In some instances, the impurities comprise terpenoid compounds, wherein one or more terpenoid compounds may be camphene, 3-carene, β-caryophyllene, caryophyllene oxide, fenchol β-myrcene, α-humulene, limonene, linalool, ocimene, α-phellandrene, α-pinene, β-pinene, terpineol, γ-terpinene, or terpinolene. In some embodiments, one or more of the impurities is a flavonoid, wherein the one or more flavonoids may be apigenin, cannflavin A, cannflavin B, kaempferol, luteolin, orientin, quercetin, or vitexin. In other instances, one or more of the impurities is a lignan, wherein the one or more lignans may be Cannabisin A, Cannabisin B, Cannabisin D, Cannabisin F, N-trans-caffeoyltyramine, N-trans-coumaroyltyramine, or N-trans-feruloyltyramine. In some embodiments, the impurities comprise cannabinoid compounds, wherein the impurities may be cannabidivarinic acid (CBDVA), cannabidinodiol (CBND), cannabigerovarinic acid (CBGVA), cannabidivarin (CBDV), cannabidiolic acid (CBDA), tetrahydrocannabivarin (THCV), cannabichromevarin (CBCV), tetrahydrocannabivarinic acid (THCVA), cannabichromevarin (CBCV), cannabinol (CBN), cannabinolic acid (CBNA), delta-9-tetrahydrocannabinol (Δ9-THC), delta-8-tetrahy drocannabinol (Δ8-THC), cannabicyclol (CBL), cannabichromene (CBC), tetrahydrocannabinolic acid (THCA), cannabichromenic acid (CBLA), or cannabinol methyl ether (CBNM). In yet other embodiments, the impurities comprise at least two of cannabidivarinic acid (CBDVA), cannabidinodiol (CBND), cannabigerovarinic acid (CBGVA), cannabidivarin (CBDV), cannabidiolic acid (CBDA), tetrahydrocannabivarin (THCV), cannabichromevarin (CBCV), tetrahydrocannabivarinic acid (THCVA), cannabichromevarin (CBCV), cannabinol (CBN), cannabinolic acid (CBNA), delta-9-tetrahydrocannabinol (Δ9-THC), delta-8-tetrahy drocannabinol (Δ8-THC), cannabicyclol (CBL), cannabichromene (CBC), tetrahydrocannabinolic acid (THCA), cannabichromenic acid (CBLA), cannabinol methyl ether (CBNM). In some embodiments, compositions disclosed herein comprise impurities at a concentration of at most 10%, or at most 5%. In some embodiments, the compositions are in a unit dose form. In yet other instances, the compositions are packaged into a container selected from the group consisting of a tube, a jar, a vial, a bag, a tray, a drum, a bottle, a syringe, and a can. In some instances, the container contains information describing directions for use.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a Store-Operated Calcium Entry (SOCE) Fura-2 bioassay in various cell types, in accordance with embodiments. Traces in black indicate full activation by appropriate agonist (Tg: Thapsigargin 1 μM) as indicated in panels. Traces labeled Gd³⁺ represent data obtained in the presence of inhibitor compound (Gd³⁺: Gadolinium Chloride 1 M).

FIG. 2 illustrates Fura-2 bioassays of over-expressed TRP ion channels involved in pain, in accordance with embodiments. Traces in black indicate full activation by appropriate agonists as indicated in panels. Traces in gray represent data obtained in the presence of inhibitor compound. Arrows indicate time of agonist application. PS: pregnenolone sulfate; AITC: allyl isothiocyanate. FIG. 2A depicts ion channel TRPM3. FIG. 2B depicts ion channel TRPM8. FIG. 2C depicts ion channel TRPA1. FIG. 2D depicts ion channel TRPV1.

FIG. 3 illustrates agonist-induced Ca²⁺ oscillations in three intact Jurkat T lymphocytes, in accordance with embodiments.

FIGS. 4A-4F illustrate whole-cell patch clamp electrophysiology of various ion channels in tetracycline-induced overexpressing HEK293 cells, in accordance with embodiments.

FIG. 4A shows activation of TRPV1, in accordance with embodiments. Left panel is averaged current development before, during and after agonist application (n=3-5, S.E.M.). Right panel is representative current-voltage traces extracted at the time of maximal current activation.

FIG. 4B shows activation of TRPM3, in accordance with embodiments. Left panel is averaged current development before, during and after agonist application (n=3-5, S.E.M.). Right panel is representative current-voltage traces extracted at the time of maximal current activation.

FIG. 4C shows activation of TRPA1, in accordance with embodiments. Left panel is averaged current development before, during and after agonist application (n=3-5, S.E.M.). Right panel is representative current-voltage traces extracted at the time of maximal current activation.

FIG. 4D shows activation of Kv1.3, in accordance with embodiments. Left panel shows averaged current development by voltage activation (Kv1.3). Right panel is representative current-voltage traces extracted at the time of maximal current activation. Right panel is representative current-voltage traces extracted at the time of maximal current activation.

FIG. 4E shows activation of I_{CRAC}, in accordance with embodiments. Left panel shows averaged current development by internal perfusion with 50 μM inositol 1,4,5-trisphosphate (IP₃). Right panel is representative current-voltage traces extracted at the time of maximal current activation.

FIG. 4F shows activation of TRPM8, in accordance with embodiments. Left panel shows an example cell activated with menthol. Right panel is representative current-voltage traces extracted at the time of maximal current activation.

FIG. 5 illustrates cytokine release in human immune cells, in accordance with embodiments.

FIG. 6 illustrates HPLC-UV (210 nm) traces of the terpene-deficient (TerpDefExt) and terpene-rich (TerpRichExt) extracts of the Cannabis plant material (NIDA Chemovar S04) and mixtures of commercial standards of terpenes and cannabinoids, in accordance with embodiments.

FIGS. 7A-7D illustrate the effect of cannabinoids on SOCE in Jurkat cells, in accordance with embodiments. Calcium signals are solicited in intact cells by applying 1 μM thapsigargin (Tg). Gadolinium (1 μM) was used as a positive control (pos ctl) of SOCE inhibition. All data are averages of three independent runs. FIG. 7A shows screening of seven THC derivatives, in accordance with embodiments. FIG. 7B shows screening of one high-THC extract, in accordance with embodiments. FIG. 7C shows screening of nine non-THC cannabinoids, in accordance with embodiments. FIG. 7D shows screening of one high-CBD extract, in accordance with embodiments.

FIGS. 8A-8D illustrates the effect of cannabinoids on SOCE in HEK293 cells, in accordance with embodiments. All data are averages of three independent runs. FIG. 8A shows screening of seven THC derivatives, in accordance with embodiments. FIG. 8B shows screening of one high-THC extract, in accordance with embodiments. FIG. 8C shows screening of non-THC cannabinoids, in accordance with embodiments. FIG. 8D shows screening of one high-CBD extract, in accordance with embodiments.

FIGS. 9A-9P illustrate dose-response behavior of cannabinoids on store-operated calcium entry (SOCE), in accordance with embodiments. All data are averages of three independent runs±SEM. FIG. 9A depicts dose-response behavior of CBGA, CBG, and a vehicle control. FIG. 9B depicts dose-response behavior of CBGVA, and CBGV. FIG. 9C depicts dose-response behavior of CBDA, CBD, CBDVA, and CBDV. FIG. 9D depicts dose-response behavior of CBCA, CBC, and CBCV. FIG. 9E depicts dose-response behavior of CBLA, and CBL. FIG. 9F depicts dose-response behavior of CBNA, CBN, CBND, and CBNM. FIG. 9G depicts dose-response behavior of THCA, delta9-THC, delta8-THC, and THCVA. FIG. 9H depicts dose-response behavior of CBGA, CBG, and a vehicle control. FIG. 9-I depicts dose-response behavior of CBGVA, and CBGV. FIG. 9J depicts dose-response behavior of CBDA, and CBD. FIG. 9K depicts dose-response behavior of CBCA, CBC, and CBCV. FIG. 9L depicts dose-response behavior of CBDVA, and CBDV. FIG. 9M depicts dose-response behavior of CBLA and CBL. FIG. 9N depicts dose-response behavior of CBNA, CBN, CBND, and CBNM. FIG. 9-O depicts dose-response behavior of THCVA and THCV. FIG. 9P depicts dose-response behavior of THCA, delta8 THC, and delta9 THC.

FIGS. 10A-10Z illustrate combinatory effect of CBGA and other cannabinoids, in accordance with embodiments. FIGS. 10A-10T were obtained in Jurkat-NFAT cells and FIGS. 10U-10Y were obtained from THP-1 cells. All the data shown here are average of three independent runs and the values are mean±SEM. FIG. 10A depicts varying ratios of CBG to CBGA. FIG. 10B depicts varying ratios of CBGV to CBGA. FIG. 10C depicts varying ratios of THCVA to CBGA. FIG. 10D depicts varying ratios of THCV to CBGA. FIG. 10E depicts varying ratios of CBGVA to CBGA. FIG. 10F depicts varying ratios of THCA to CBGA. FIG. 10G depicts varying ratios of CBNA to CBGA. FIG. 10H depicts varying ratios of CBN to CBGA. FIG. 10-I depicts varying ratios of CBCA to CBGA. FIG. 10J depicts varying ratios of CBD to CBGA. FIG. 10K depicts varying ratios of CBND to CBGA. FIG. 10L depicts varying ratios of CBL to CBGA. FIG. 10M depicts varying ratios of CBDA to CBGA. FIG. 10N depicts varying ratios of CBDVA to CBGA. FIG. 10-O depicts varying ratios of delta8 THC to CBGA. FIG. 10P depicts varying ratios of delta9 THC to CBGA. FIG. 10Q depicts varying ratios of CBDV to CBGA. FIG. 10R depicts varying ratios of CBLA to CBGA. FIG. 10S depicts varying ratios of CBC to CBGA. FIG. 10T depicts varying ratios of CBCV to CBGA. FIGS. 10U-10Y were obtained from THP-1 cells. FIG. 10U depicts varying ratios of CBDA to CBGA. FIG. 10V depicts varying ratios of CBGVA to CBGA. FIG. 10W depicts varying ratios of THCA to CBGA. FIG. 10X depicts varying ratios of THCVA to CBGA. FIG. 10Y depicts varying ratios of CBNA to CBGA. FIG. 10Z depicts % SOC inhibition for various ratios of cannabinoids and CBGA.

FIG. 11A-FIG. 11-SS show store-operated calcium entry (SOCE) dose response curves in human cells for various hemp extracts under heated or unheated conditions, in accordance with embodiments. FIGS. 11A-11-I depict store-operated calcium entry (SOCE) dose response curves in HEK293 cells for various hemp extracts under heated or unheated conditions.

FIG. 11A depicts dose response curves for hemp variety CW. FIG. 11B depicts dose response curves for hemp variety LIF. FIG. 11C depicts dose response curves for hemp variety WCBG. FIG. 11D depicts dose response curves for hemp variety ELEK. FIG. 11E depicts dose response curves for hemp variety SH. FIG. 11F depicts dose response curves for hemp variety SSC. FIG. 11G depicts dose response curves for hemp variety GS. FIG. 11H depicts dose response curves for hemp variety SS. FIG. 11-I depicts dose response curves for hemp variety HH.

FIGS. 11J-11R depict store-operated calcium entry (SOCE) dose response curves in Jurkat cells for various hemp extracts under heated or unheated conditions. FIG. 11J depicts dose response curves for hemp variety CW. FIG. 11K depicts dose response curves for hemp variety HH.

FIG. 11L depicts dose response curves for hemp variety SSC. FIG. 11M depicts dose response curves for hemp variety ELEK. FIG. 11N depicts dose response curves for hemp variety LIF. FIG. 11-O depicts dose response curves for hemp variety SS. FIG. 11P depicts dose response curves for hemp variety GS. FIG. 11Q depicts dose response curves for hemp variety SH. FIG. 11R depicts dose response curves for hemp variety WCBG.

FIGS. 11S-11AA depict store-operated calcium entry (SOCE) dose response curves in LUVA cells for various hemp extracts under heated or unheated conditions. FIG. 11S depicts dose response curves for hemp variety CW. FIG. 11T depicts dose response curves for hemp variety HH. FIG. 11U depicts dose response curves for hemp variety SSC. FIG. 11V depicts dose response curves for hemp variety ELEK. FIG. 11W depicts dose response curves for hemp variety LIF. FIG. 11X depicts dose response curves for hemp variety SS. FIG. 11Y depicts dose response curves for hemp variety GS. FIG. 11Z depicts dose response curves for hemp variety SH. FIG. 11AA depicts dose response curves for hemp variety WCBG.

FIGS. 11BB-11JJ depict store-operated calcium entry (SOCE) dose response curves in RBL2H3 cells for various hemp extracts under heated or unheated conditions. FIG. 11BB depicts dose response curves for hemp variety CW. FIG. 11CC depicts dose response curves for hemp variety HH. FIG. 11DD depicts dose response curves for hemp variety SSC. FIG. 11EE depicts dose response curves for hemp variety ELEK. FIG. 11FF depicts dose response curves for hemp variety LIF. FIG. 11GG depicts dose response curves for hemp variety SS. FIG. 11HH depicts dose response curves for hemp variety GS. FIG. 11-II depicts dose response curves for hemp variety SS. FIG. 11JJ depicts dose response curves for hemp variety WCBG.

FIGS. 11KK-11SS depict store-operated calcium entry (SOCE) dose response curves in U937 cells for various hemp extracts under heated or unheated conditions. FIG. 11KK depicts dose response curves for hemp variety CW. FIG. 11LL depicts dose response curves for hemp variety HH. FIG. 11MM depicts dose response curves for hemp variety SSC. FIG. 11NN depicts dose response curves for hemp variety ELEK. FIG. 11-OO depicts dose response curves for hemp variety LIF. FIG. 11PP depicts dose response curves for hemp variety SS. FIG. 11QQ depicts dose response curves for hemp variety GS. FIG. 11RR depicts dose response curves for hemp variety SH. FIG. 11SS depicts dose response curves for hemp variety WCBG.

FIG. 11TT-FIG. 11-XX show store-operated calcium entry (SOCE) dose response curves in human cells for various hemp extracts, in accordance with embodiments. FIG. 11TT depicts SOCE dose response curves with Jurkat cells. FIG. 11UU depicts SOCE dose response curves with Luva cells. FIG. 11VV depicts SOCE dose response curves with RBL2H3 cells. FIG. 11-WW depicts SOCE dose response curves with U937 cells. FIG. 11-XX depicts SOCE dose response curves with HEK293 cells.

FIG. 12 illustrates the effect of CBGA in blocking Ca²⁺ Release-activated Ca²⁺ inward current (I_{CRAC}), in accordance with embodiments.

FIG. 13 illustrates the effect of CBGA on inward current and outward currents at −120 mV and +40 mV, respectively. CBGA blocks inward currents carried by Ca²⁺ Release-activated Ca²⁺ (CRAC) channels (gray symbols) in parallel with outward currents (black symbols) carried by TRPM7 (Transient receptor potential cation channel, subfamily M, member 7), in accordance with embodiments.

FIG. 14 illustrates activation of TRPM7 over-expressed in HEK293 cells by perfusing cell with intracellular solution containing 0 ATP and 0 Mg²⁺, resulting in fast and maximal activation of TRPM7 outward currents at +40 mV. CBGA dose-dependently inhibits TRPM7 currents, in accordance with embodiments.

FIG. 15 illustrates dose-response curves for the inhibition of TRPM7 currents (dark gray symbols) obtained in FIG. 14 and SOCE-mediated increases in intracellular Ca²⁺ (light gray symbols), in accordance with embodiments.

FIG. 16 illustrates the measured body weights of mice in UUO mouse experiments, in accordance with embodiments. Black circles represent the vehicle treatment control group, light gray circles are CBGA treatment group, medium gray circles are CBD treatment group and dark gray circles are CBGA+CBD treatment group.

FIG. 17A depicts representative contralateral kidney (CLK) (Left side) and UUO kidneys (Right side) isolated from UUO mice at day 7 after ureteral obstruction surgery, in accordance with embodiments. Scale bars represent 5 mm.

FIG. 17B illustrates the weight of UUO kidney at day 7, in accordance with embodiments. From left to right, bars represent vehicle control group, CBGA treatment group, CBD treatment group, and CBGA+CBD treatment group. *p<0.05, **p<0.01 vs. vehicle UUO kidneys.

FIG. 18 illustrates the output of magnesium in urine is reduced in unilateral ureteral obstruction (UUO) mice treated with cannabinoids, in accordance with embodiments.

FIG. 19A shows representative pictures of HE staining taken from CLK and UUO kidney sections (magnification ×200), in accordance with embodiments. Scale bars, 100 μm.

FIG. 19B illustrates the number of dilated tubules assessed in one representative field. White bars represent CLK kidneys and black bars represent UUO kidneys, in accordance with embodiments. **p<0.01 vs. CLK kidneys, ##p<0.01 vs. vehicle UUO kidneys.

FIG. 19C illustrates the total number of renal tubules assessed in one representative field, in accordance with embodiments. **p<0.01 vs. CLK kidneys, ##p<0.01 vs. vehicle UUO kidneys.

FIG. 19D illustrates the assessment of interstitial area in one representative field, in accordance with embodiments. *p<0.05, **p<0.01 vs. CLK kidneys, ##p<0.01 vs. vehicle UUO kidneys.

FIG. 20A depicts representative pictures of immunostainings for F4/80 as a marker of macrophage in CLK (upper panels) and UUO kidneys (lower panels, magnification ×200) Vehicle or cannabinoid treatment, in accordance with embodiments. Scale bars are 100 m.

FIG. 20B illustrates the number of macrophages counted in CLK kidneys (white bars) and UUO kidneys (black bar), in accordance with embodiments. **p<0.01 vs. CLK kidneys, ##p<0.01 vs. vehicle UUO kidneys. ††p<0.01 vs. vehicle CLK kidneys.

FIG. 21A depicts representative pictures of immunostainings for Collagen type I as a marker of macrophage in CLK (upper panels) and UUO kidneys (lower panels, magnification ×200) Vehicle or cannabinoid treatment, in accordance with embodiments. Scale bars are 100 μm.

FIG. 21B illustrates the average percentage of the Collagen type I-positive area in CLK kidneys (white bars) and UUO kidneys (black bar), in accordance with embodiments. The staining intensity in the interstitium was computed using Image J software. **p<0.01 vs. CLK kidneys, ##p<0.01 vs. vehicle UUO kidneys.

FIG. 22A shows representative pictures of immunostainings for fibronectin in CLK (top panels) and UUO kidneys (lower panels, magnification ×200) with or without cannabinoid treatment, in accordance with embodiments. Scale bars are 100 m.

FIG. 22B illustrates graph plots of the average percentage of the fibronectin-positive area in kidneys in CLK (white bar) and UUO (black bar) kidneys, in accordance with embodiments. The staining intensity in the interstitium was computed using Image J software. **p<0.01 vs. CLK kidneys. ##p<0.01, vs. vehicle UUO kidneys.

FIG. 23 shows representative images of Western blotting assay results showing expression of α-SMA and phosphorylated Smad3 in UUO kidneys treated with vehicle or cannabinoid extracts, in accordance with embodiments.

FIG. 24A shows representative micrograph images of α-SMA immunostaining in CLK (top panels) and UUO kidneys (lower panels, magnification ×200) with or without cannabinoid treatment, in accordance with embodiments. Scale bars are 100 μm.

FIG. 24B shows quantification of average α-SMA-positive area in CLK (white bar) and UUO (black bar) kidneys from UUO experiments, in accordance with embodiments. *p<0.05, **p<0.01 vs. CLK kidneys. #p<0.05, ##p<0.01, vs. vehicle UUO kidneys.

FIG. 25 shows measured body weights of mice during cisplatin nephritis model experiments, in accordance with embodiments.

FIGS. 26A-26B show kidney weights, in accordance with embodiments described herein. FIG. 26A shows weight of the left kidney. FIG. 26B shows weight of the right kidney.

FIGS. 27A-27B show quantification of magnesium in blood serum and urine from cisplatin-induced nephritic mice, in accordance with embodiments. FIG. 27A shows measured magnesium concentrations in serum. FIG. 27B shows magnesium concentrations in urine. p<0.05, **p<0.01 vs. cis(+) vehicle treatment group.

FIG. 28 shows quantification of kidney function in cisplatin-induced mouse experiments using blood urea nitrogen (BUN) measurement, in accordance with embodiments. **p<0.01 vs. cis(+) vehicle treatment group.

FIGS. 29A-29B shows quantification of creatinine in serum and urine from cisplatin-induced nephritic mice, in accordance with embodiments. FIG. 29A shows measured creatine concentrations in serum. FIG. 29B shows creatinine concentrations in urine. *p<0.05 vs. cis(+) vehicle treatment group.

FIGS. 30A-30B show evaluation of apoptosis in cisplatin-induced mouse nephritis experiments, in accordance with embodiments. FIG. 30A shows representative Western blotting assay images for full-length PARP-1 in accordance with embodiments. FIG. 30B shows densitometric analysis of PARP-1 protein bands from Western blotting images (n=4-5). *p<0.05, **p<0.01 vs. cis(+) vehicle treatment group.

FIGS. 31A-31G show mRNA expression of cytokines and inflammatory related proteins in kidneys analyzed in cisplatin nephritis mouse model experiments. Shown in these figures are mRNA expression levels of tumor necrosis factor alpha (TNF-α)(FIG. 31A), interleukin 6 (IL-6) (FIG. 31B), C-X-C motif chemokine ligand 10 (CxCl 10) (FIG. 31C), intercellular adhesion molecule 1 (ICAM-1) (FIG. 31D), monocyte chemoattractant protein-1 (MCP-1) (FIG. 31E), C-reactive protein (CRP) (FIG. 31F), and endothelin-1 (ET-1) (FIG. 31G), in accordance with embodiments. *p<0.05, **p<0.01 vs. cis(+) vehicle treatment group.

### DETAILED DESCRIPTION

The present disclosure relates to compositions, methods, and systems comprising analgesic, anti-inflammatory, phytochemicals derived from the Cannabis plant and methods of treatment using the same. Cannabis sativa has two major classes of compounds: cannabinoid and terpenoid compounds. Terpenes represent one of the largest classes of natural products with greater than 55,000 known compounds and have a range of pharmacological properties that include anticancer, antimicrobial, antifungal, antiviral, antihyperglycemic, antiparasitic, anti-inflammatory, and analgesic effects. Similarly, cannabinoids have been reported to exhibit a wide range of biological effects, including some efficacy in the treatment of pain, chemotherapy-induced nausea and vomiting.

Cannabinoid drugs are presently used as analgesics, but experimental pain studies have produced mixed results with regards to analgesic activity of cannabinoids, particularly with respect to neuropathic pain. The two main cannabinoids in Cannabis, the psychoactive Δ9-tetrahydrocannabinol (Δ9-THC) and the non-psychoactive cannabidiol (CBD), are both available in the United States as therapeutics. Marinol™ is a soft gelatin capsule containing Δ9-THC dissolved in sesame oil to treat nausea and vomiting associated with cancer chemotherapy in patients who have failed to respond adequately to conventional therapies. Epidiolex® is an oral solution comprising purified CBD for treating seizures associated with 2 rare forms of epilepsy Dravet and Lennox-Gastaut Syndromes. Sativex® is a specific extract of Cannabis containing equal amounts of THC and CBD that was approved as a botanical drug in the United Kingdom in 2010 as a mouth spray to alleviate neuropathic pain, spasticity, overactive bladder, and other symptoms of multiple sclerosis.

The present disclosure describes high-throughput assays used to assess the efficacy and potency of various Cannabis phytochemicals—alone or in combination—in suppressing the pro-inflammatory activity of key immune cell types involved in inflammatory pain. The disclosure describes the characterization of relevant targets affected by various, non-hallucinatory, Cannabis phytochemicals and their analgesic properties in animal models of inflammatory nociceptive and neuropathic pain.

### Phytochemicals of the Cannabis Plant

Described herein are compositions present in the Cannabis plant (e.g., C. sativa). Such compounds may be categorized as cannabinoids. Exemplary cannabinoids, without limitation, are described in Table 1. In some embodiments, cannabinoids are extracted or otherwise obtained from plants such as Cannabis spp. (i.e., “plant based”). In some embodiments, cannabinoids are synthesized using chemical synthesis, recombinant biosynthesis, or a combination of both.

Cannabinoids in some instances comprise a diverse array of chemical functional groups or structural shapes which influence their biological activity. For example, acidic cannabinoids in some instances comprise at least one carboxylic acid group. Acidic cannabinoids include but are not limited to cannabidivarinic acid, cannabigerovarinic acid, cannabidiolic acid, cannabigerolic acid, tetrahydrocannabivarinic acid, cannabinolic acid, tetrahydrocannabinolic acid, cannabichromenic acid, or cannabicyclolic acid. In some embodiments, cannabinoids comprise one, two, three, or more than three chemical ring systems.

Table 1 describes exemplary cannabinoids.

| Cmpd | | | | |
|---|---|---|---|---|
| # | Abbrev. | Name | Structure | Chemical name |
| 1 | CBDVA | cannabidivarinic acid | 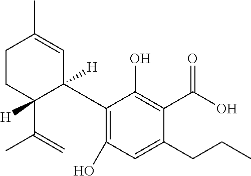 [SMILES: C=C(C)[C@@H]1CCC(C)=C[C@H]1c1c(O)cc(CCC)c(C(=O)O)c1O] | (1′R,2′R)-2,6- dihydroxy-5′-methyl-2′- (prop-1-en-2-yl)-4- propyl-1′,2′,3′,4′- tetrahydro-[1,1′- biphenyl]-3-carboxylic acid |
| 2 | CBND | cannabidinodiol | 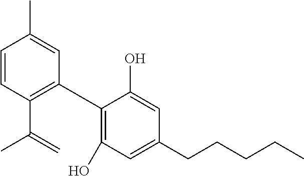 [SMILES: C=C(C)c1ccc(C)cc1-c1c(O)cc(CCCCC)cc1O] | 5′-methyl-4-pentyl-2′- (prop-1-en-2-yl)-[1,1′ biphenyl]-2,6-diol |
| 3 | CBGVA | cannabigerovarinic acid | 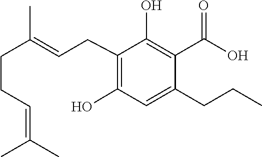 [SMILES: CCCc1cc(O)c(C/C=C(\C)CCC=C(C)C)c(O)c1C(=O)O] | (E)-3-(3,7- dimethylocta-2,6-dien- 1-yl)-2,4-dihydroxy-6- propylbenzoic acid |
| 4 | CBDV | cannabidivarin | 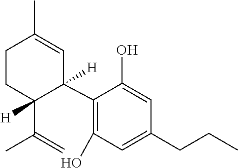 [SMILES: C=C(C)[C@@H]1CCC(C)=C[C@H]1c1c(O)cc(CCC)cc1O] | (1′R,2′R)-5′-methyl-2′- (prop-1-en-2-yl)-4- propyl-1′,2′,3′,4′- tetrahydro-[1,1′- biphenyl]-2,6-diol |
| 5 | CBDA | cannabidiolic acid | 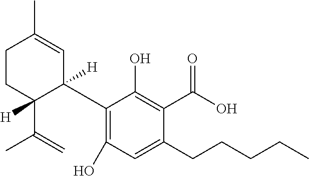 [SMILES: C=C(C)[C@@H]1CCC(C)=C[C@H]1c1c(O)cc(CCCCC)c(C(=O)O)c1O] | (1′R,2′R)-2,6- dihydroxy-5′-methyl-4- penlyl-2′-(prop-1-en-2- yl)-1′,2′,3′,4′-tetrahydro- [1,1′-biphenyl]-3- carboxylic acid |
| 6 | CBGA | cannabigerolic acid | 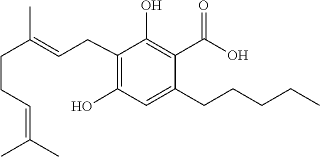 [SMILES: CCCCCc1cc(O)c(C/C=C(\C)CCC=C(C)C)c(O)c1C(=O)O] | (E)-3-(3,7- dimethylocta-2,6-dien- 1-yl)-2,4-dihydroxy-6- pentylbenzoic acid |
| 7 | CBG | cannabigerol | 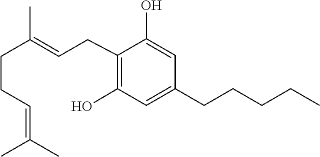 [SMILES: CCCCCc1cc(O)c(C/C=C(\C)CCC=C(C)C)c(O)c1] | (E)-2-(3,7- dimethylocta-2,6-dien- 1-yl)-5-penlylbenzene- 1,3-diol |
| 8 | CBD | cannabidiol | 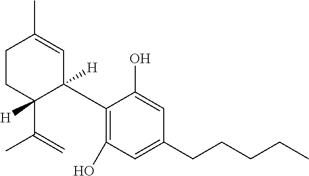 [SMILES: C=C(C)[C@@H]1CCC(C)=C[C@H]1c1c(O)cc(CCCCC)cc1O] | (1′R,2′R)-5′-methyl-4- penlyl-2′-(prop-1-en-2- yl)-1′,2′,3′,4′-tetrahydro- [1,1′-biphenyl]-2,6-diol |
| 9 | THCV | tettabydrocannabivarin | 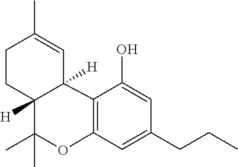 [SMILES: CCCc1cc(O)c2c(c1)OC(C)(C)[C@@H]1CCC(C)=C[C@@H]21] | trimethyl-3-propyl- 6a,7,8,10a-tetrahydro- 6H-benzo[c]chromen-1- ol |
| 10 | CBCV | cannabichromevarin | 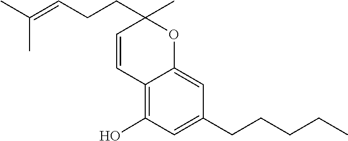 [SMILES: CCCCCc1cc(O)c2c(c1)OC(C)(CCC=C(C)C)C=C2] | 2-methyl-2-(4- methylpent-3-en-1-yl)- 7-pentyl-2H-chromen- 5-ol |
| 11 | THCVA | tetrahydrocannabivarinic acid | 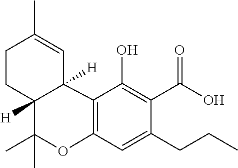 [SMILES: CCCc1cc2c(c(O)c1C(=O)O)[C@@H]1C=C(C)CC[C@H]1C(C)(C)O2] | 6,6,9-trimethyl-3- propyl-6a,7,8,10a- tetrahydro-6H- benzo[c]chromene-2- carboxylic acid |
| 12 | CBN | cannabinol | 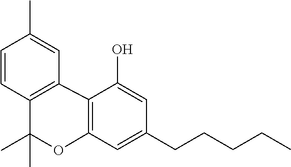 [SMILES: CCCCCc1cc(O)c2c(c1)OC(C)(C)c1ccc(C)cc1-2] | 6,6,9-trimethyl-3- pentyl-6H- benzo[c]chromen-1-ol |
| 13 | CBNA | cannabinolic acid | 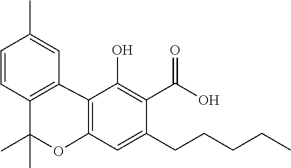 [SMILES: CCCCCc1cc2c(c(O)c1C(=O)O)-c1cc(C)ccc1C(C)(C)O2] | 1-hydroxy-6,6,9- trimethyl-3-pentyl-6H- benzo[c]chromene-2- carboxylic acid |
| 14 | Δ9-THC | delta-9- tetrahydrocannabinol | 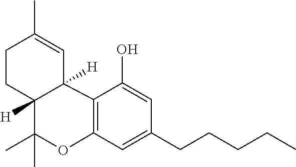 [SMILES: CCCCCc1cc(O)c2c(c1)OC(C)(C)[C@@H]1CCC(C)=C[C@@H]21] | (6aR,10aR)-6,6,9- trimethyl-3-pentyl- 6a,7,8,10a-tetrahydro- 6H-benzo[c]chromen-1- ol |
| 15 | Δ8-THC | delta-8- tetrahydrocannabinol | 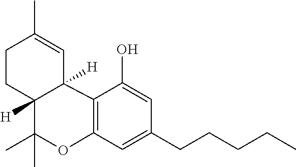 [SMILES: CCCCCc1cc(O)c2c(c1)OC(C)(C)[C@@H]1CCC(C)=C[C@@H]21] | (6aR,10aR)-6,6,9- trimethyl-3-pentyl- 6a,7,10,10a-tetrahydro- 6H-benzo[c]chromen-1- ol |
| 16 | CBL | cannabicyclol | 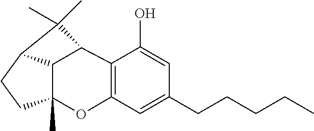 | (1aS,1a1R,3aR,8bR)- 1,1,3a-trimethyl-6- pentyl-1a,1a1,2,3,3a,8b- hexahydro-1H-4- oxabenzo[f]cyclobuta[c d]inden-8-ol |
| 17 | CBC | cannabichromene | 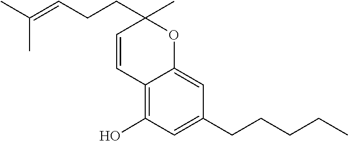 [SMILES: CCCCCc1cc(O)c2c(c1)OC(C)(CCC=C(C)C)C=C2] | 2-methyl-2-(4- methylpent-3-en-1-yl)- 7-pentyl-2H-chromen- 5-ol |
| 18 | THCA | tetrahydrocannabinolic acid | 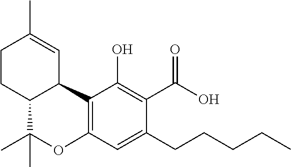 [SMILES: CCCCCc1cc2c(c(O)c1C(=O)O)[C@@H]1C=C(C)CC[C@H]1C(C)(C)O2] | (6aR, 10aR)-1-hydroxy- 6,6,9-trimethyl-3- pentyl-6a,7,8,10a- tetrahydro-6H- benzo[c]chromene-2- carboxylic acid |
| 19 | CBCA | cannabichromenic acid | 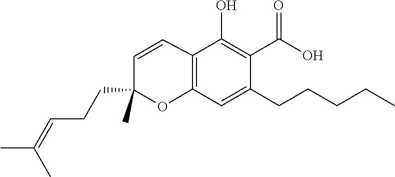 [SMILES: CCCCCc1cc2c(c(O)c1C(=O)O)C=C[C@@](C)(CCC=C(C)C)O2] | (R)-5-hydroxy-2- methyl-2-(4- methylpent-3-en-1-yl)- 7-pentyl-2H-chromene- 6-carboxylic acid |
| 20 | CBLA | cannabicyclolic acid | 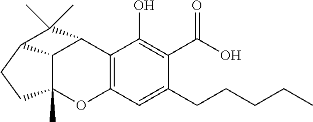 | (1aS,1a1R,3aR,8bR)-8- hydroxy-1,1,3a- trimethyl-6-pentyl- 1a, 1a1, 2,3,3a, 8b- hexahydro-1H-4- oxabenzo[f]cyclobuta[c d]indene-7-carboxylic acid |
| 21 | CBNM | cannabinol methyl ether | 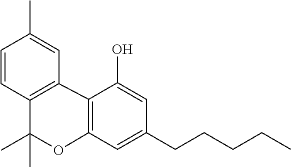 [SMILES: CCCCCc1cc(O)c2c(c1)OC(C)(C)c1ccc(C)cc1-2] | 1-methoxy-6,6,9- trimethyl-3-pentyl-6H- benzo[c]chromene |
| 22 | CBGV | cannabigerovarin | 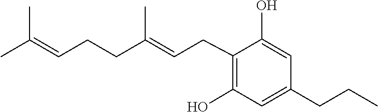 [SMILES: CCCc1cc(O)c(C/C=C(\C)CCC=C(C)C)c(O)c1] | (E)-2-(3,7- dimethylocta-2,6-dien- 1-yl)-5-propylbenzene- 1,3-diol |
| 23 | 8β-OH-Δ9 THC | 8β-hydroxy-Δ9 tetrahydrocannabinol | 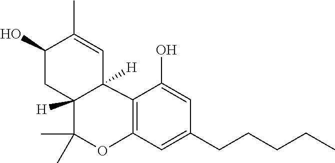 [SMILES: CCCCCc1cc(O)c2c(c1)OC(C)(C)[C@@H]1C[C@@H](O)C(C)=C[C@@H]21] | (6aR,8R,10aR)-6,6,9- trimethyl-3-pentyl- 6a,7,8,10a-tetrahydro- 6H-benzo[c]chromene- 1,8-diol |
| 24 | 11-OH-Δ9 THC | 11-hydroxy-Δ9 tetrahydrocannabinol | 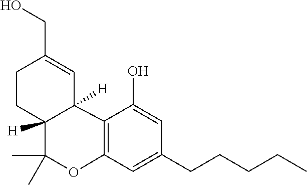 [SMILES: CCCCCc1cc(O)c2c(c1)OC(C)(C)[C@@H]1CCC(CO)=C[C@@H]21] | (6aR,10aR)-9- (hydroxymethyl)-6,6- dimethyl-3-pentyl- 6a,7,8,10a-tetrahydro- 6H-benzo[c]chromen-1- ol |
| 25 | 11- COOH- Δ9-THC | 11-nor-9-carboxy- Δ9- tetrahydrocannabinol | 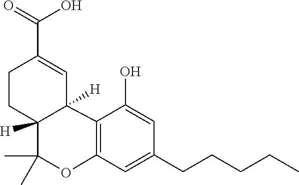 [SMILES: CCCCCc1cc(O)c2c(c1)OC(C)(C)[C@@H]1CCC(C(=O)O)=C[C@@H]21] | (6aR,10aR)-1-hydroxy- 6,6-dimethyl-3-pentyl- 6a,7,8,10a-tetrahydro- 6H-benzo[c]chromene- 9-carboxylic acid |

### Sources of Cannabinoids

Extraction of Cannabinoids from Plant Source

Cannabinoids may be obtained as an extract from plant-based materials, such as Cannabis spp or from organisms genetically modified to recombinantly synthesize them. The Cannabis spp plant extract source may be plant material from regulated sources, for example, the National Institute on Drug Abuse (NIDA), or from hemp (obtained from various vendors, including Berkshire CBD, Plain Jane, Earth Matters, Ventura Seed Company), which by definition comprises low to negligible levels of THC. Such extracts are in some instances used directly as pharmaceutical compositions. In some embodiments, extracts may be exposed to elevated temperatures, as disclosed herein.

Cannabinoids and extracts thereof may be combined with additional components. In some embodiments, additional components are added to such extracts. In some embodiments, extracts comprise increased amounts of desired cannabinoids (e.g., cannabidiolic acid), and decreased amounts of undesired cannabinoids, or other impurity. Amounts of impurities may be measured by any method known in the art. In some embodiments, the amount of impurities is measured using HPLC, GC, GC/MS, NMR or other analytical method. In some embodiments, purity is measured against a standard sample of known purity. The commercial standards of the cannabinoids, terpenes, flavonoids and other phytochemicals of Cannabis spp are obtained from various chemical vendors, including Cayman Chemical Company, Sigma-Aldrich, NIDA, etc. In some embodiments, extracts comprise at most 50%, 45%, 40%, 35%, 30%, 25%, 20%, 15%, 10%, or at most 5% (w/w) impurities. In some embodiments, extracts comprise about 50%, 45%, 40%, 35%, 30%, 25%, 20%, 15%, 10%, or about 5% (w/w) impurities. In some embodiments, extracts comprise 1-2%, 1-5%, 1-15%, 2-10%, 2-15%, 5-10%, 5-20%, 10-25%, or 5-25% (w/w) impurities.

Extracts may comprise one or more cannabinoids. In some embodiments, extracts comprise no more than 99%, 98%, 95%, 90%, 85%, 80%, 75%, 70%, 65%, 60%, 55%, or no more than 50% cannabinoids. In some embodiments, extracts comprise no more than 99%, 98%, 95%, 90%, 85%, 80%, 75%, 70%, 65%, 60%, 55%, or no more than 50% cannabidiolic acid (CBDA) or cannabigerolic acid (CBGA). In some embodiments, extracts comprise 50-99%, 50-98%, 50-95%, 50-90%, 50-85%, 20-95%, 30-90%, 50-80%, or 50-50% cannabidiolic acid (CBDA) or cannabigerolic acid (CBGA). In some embodiments, extracts comprise no more than 99%, 98%, 95%, 90%, 85%, 80%, 75%, 70%, 65%, 60%, 55%, or no more than 50% cannabidiolic acid (CBDA) and cannabigerolic acid (CBGA). In some embodiments, extracts comprise no more than 99%, 98%, 95%, 90%, 85%, 80%, 75%, 70%, 65%, 60%, 55%, or no more than 50% cannabidiolic acid (CBDA), cannabigerolic acid (CBGA), or cannabigerol (CBG). In some embodiments, extracts comprise no more than 99%, 98%, 95%, 90%, 85%, 80%, 75%, 70%, 65%, 60%, 55%, or no more than 50% cannabidiolic acid (CBDA), cannabigerolic acid (CBGA), and cannabigerol (CBG). In some embodiments, extracts comprise 50-99%, 50-98%, 50-95%, 50-90%, 50-85%, 20-95%, 30-90%, 50-80%, or 50-50% cannabidiolic acid (CBDA), cannabigerolic acid (CBGA), and cannabigerol (CBG). In some embodiments, extracts comprise no more than 99%, 98%, 95%, 90%, 85%, 80%, 75%, 70%, 65%, 60%, 55%, or no more than 50% cannabidiolic acid (CBDA), cannabigerolic acid (CBGA), cannabigerol (CBG), or cannabidiol (CBD). In some embodiments, extracts comprise no more than 99%, 98%, 95%, 90%, 85%, 80%, 75%, 70%, 65%, 60%, 55%, or no more than 50% cannabidiolic acid (CBDA), cannabigerolic acid (CBGA), cannabigerol (CBG), and cannabidiol (CBD). In some embodiments, extracts comprise 50-99%, 50-98%, 50-95%, 50-90%, 50-85%, 20-95%, 30-90%, 50-80%, or 50-50% cannabidiolic acid (CBDA), cannabigerolic acid (CBGA), cannabigerol (CBG), and cannabidiol (CBD).

Extracts may comprise one or more additional impurities. Such impurities include but are not limited to non-cannabinoid terpenes, flavonoids, lignans, or other cannabinoids. In some embodiments, terpenes comprise camphene, 3-carene, β-caryophyllene, caryophyllene oxide, fenchol, β-myrcene, α-humulene, limonene, linalool, ocimene, α-phellandrene, α-pinene, 3-pinene, terpineol, γ-terpinene, or terpinolene. In some embodiments, flavonoids comprise apigenin, cannflavin A, cannflavin B, kaempferol, luteolin, orientin, quercetin, or vitexin. In some embodiments, lignans comprise Cannabisin A, Cannabisin B, Cannabisin D, Cannabisin F, N-trans-caffeoyltyramine, N-trans-coumaroyltyramine, or N-trans-feruloyltyramine. In some embodiments, cannabinoid impurities comprise at least one of cannabidivarinic acid (CBDVA), cannabidinodiol (CBND), cannabigerovarinic acid (CBGVA), cannabidivarin (CBDV), cannabidiolic acid (CBDA), tetrahydrocannabivarin (THCV), cannabichromevarin (CBCV), tetrahydrocannabivarinic acid (THCVA), cannabichromevarin (CBCV), cannabinol (CBN), cannabinolic acid (CBNA), delta-9-tetrahydrocannabinol (Δ9-THC), delta-8-tetrahydrocannabinol (Δ8-THC), cannabicyclol (CBL), cannabichromene (CBC), tetrahydrocannabinolic acid (THCA), cannabichromenic acid (CBLA), cannabinol methyl ether (CBNM). In some embodiments, cannabinoid impurities comprise at least two of cannabidivarinic acid (CBDVA), cannabidinodiol (CBND), cannabigerovarinic acid (CBGVA), cannabidivarin (CBDV), cannabidiolic acid (CBDA), tetrahydrocannabivarin (THCV), cannabichromevarin (CBCV), tetrahydrocannabivarinic acid (THCVA), cannabichromevarin (CBCV), cannabinol (CBN), cannabinolic acid (CBNA), delta-9-tetrahydrocannabinol (Δ9-THC), delta-8-tetrahydrocannabinol (Δ8-THC), cannabicyclol (CBL), cannabichromene (CBC), tetrahydrocannabinolic acid (THCA), cannabichromenic acid (CBLA), cannabinol methyl ether (CBNM). In some instances, compositions described herein comprise no more than 1, 2, 3, 4, 5, 6, 7, or 8 impurities.

Extracted cannabinoids may be purified to a known purity. In some embodiments, the purified extracted cannabinoids are cannabidiolic acid (CBDA) or cannabigerolic acid (CBGA). In some embodiments, the extracted cannabinoids are purified such that it comprises at least 50%, 55%, 60%, 65%, 70%, 75%, 80%, 85%, 90%, 91%, 92%, 93%, 94%, 95%, 96%, 97%, 98%, 99% or at least 99.5% (w/w) of the desired cannabinoid. In some embodiments, the extracted cannabinoids are purified such that it comprises no more than 50%, 45%, 40%, 35%, 30%, 25%, 20%, 15%, 10%, or about 5% (w/w) of other cannabinoids. In some embodiments, extracts comprise about 1-2%, 1-5%, 1-15%, 2-10%, 2-15%, 5-10%, 5-20%, 10-25%, or 5-25% (w/w) of other cannabinoids.

Chemical or Biological Synthesis

Compositions described herein may comprise cannabinoids created synthetically (i.e., “synthetic” cannabinoids). Such synthesis methods include chemical synthesis or biological synthesis (e.g., recombinant expression of biosynthetic pathways). In some embodiments, cannabinoids are generated using a combination of chemical and biosynthetic methods (e.g., semi-synthesis). Chemical methods of cannabinoid synthesis are described in Shultz et al. Org. Lett. 2018, 20, 2381-384, and references cited therein. In some embodiments, cannabinoids are recombinantly expressed in a host organism such as a eukaryote or prokaryote. In some embodiments, cannabinoids are recombinantly expressed in a host organism such as a eukaryote cell or prokaryote cell. In some embodiments the host organism is a non-Cannabis plant, such as a tobacco plant or an insect cell. In some the host organism is a microorganism. In some the host organism is yeast. In some the host organism is E. coli. In some embodiments the host organism is not a human. Recombinant methods of cannabinoid synthesis are described in Carvalho et al. FEMS Yeast Res. 2017, 17(4), 1, and references cited therein.

### Temperature Lability

Compositions described herein may comprise temperature labile compounds, wherein exposure to heat or elevated temperature causes structural changes in the compounds. In some instances, control of temperature during processing of compositions (e.g., extraction or other process) influences the chemical composition of the resulting extract or product. Structural changes variously comprise isomerization of bonds, elimination reactions, substitution, ring formation, ring opening, or other chemical reactions. The rate of change and amount of temperature-modified product for such compounds in some instances depends on both temperature and time the compound is exposed to a given temperature. In some instances, compositions or compounds are treated with heat to effect chemical changes in the compounds thereof. Such changes in some embodiments increase the amount of desired compounds and/or decrease the amount of undesired compounds.

In some embodiments, processes are conducted at a temperature of less than 120, 110, 100, 90, 80, 70, 60, 55, 50, 45, 40, 35, 30, 25, 20, 15, 10, or less than 5° C. In some embodiments, processes are conducted at a temperature of about 120, 110, 100, 90, 80, 70, 60, 55, 50, 45, 40, 35, 30, 25, 20, 15, 10, or less than 5° C. In some embodiments, processes are conducted at a temperature of 100-120, 75-120, 10-120, 10-110, 10-100, 10-90, 20-80, 30-70, 40-60, 20-60, 30-50, 25-50, 10-45, 10-50, 20-50, 20-45, or 5-50° C. In some embodiments, extracts comprising one or more of CBGA, CBGVA, THCA, THCVA, CBDA, CBDVA, CBCA, and/or CBCVA is heated. In some embodiments, heat treatment of such an extract results in enrichment of THC, THCV, CBD, CBDV, CBC, CBCV, CBG, and/or CBGV. In some embodiments, a composition is exposed to an elevated temperature by heating for at least 1, 2, 5, 10, 12, 15, 20, 30, 45, or at least 60 seconds. In some embodiments, a composition is exposed to an elevated temperature (such as above 20° C.) by heating for at least 1, 2, 5, 10, 12, 15, 20, 30, 45, or at least 60 minutes. In some embodiments, a composition is exposed to an elevated temperature by heating for at least 1, 2, 5, 10, 12, 15, 20, 30, 45, or at least 60 hours. In some embodiments, a composition is exposed to an elevated temperature by heating for 1-5, 1-10, 2-5, 2-10, 8-15, 10-20, 10-15, 20-30, 20-45, or 30-60 seconds. In some embodiments, a composition is exposed to an elevated temperature by heating for 1-2, 1-5, 1-10, 2-5, 2-10, 8-15, 10-20, 10-15, 20-30, 20-45, or 30-60 minutes. In some embodiments, a composition is exposed to an elevated temperature by heating for 1-2, 1-5, 1-10, 2-5, 2-10, 8-15, 10-20, 10-15, 20-30, 20-45, or 30-60 hours.

### Synergistic Combinations

Described herein are compositions, such as pharmaceutical compositions comprising two or more chemical compounds. In some embodiments, a first chemical compound is a cannabinoid. In some embodiments, a first chemical compound and a second chemical compound are each cannabinoids. In some embodiments, the first cannabinoid is an acidic cannabinoid. Combinations of two or more cannabinoids in some embodiments produce additive, sub-additive, supra-additive, or entourage biological effects. In some embodiments an additive effect is measured by combination indices (CI) according to the method of isoboles. In some embodiments a supra-additive effect is measured by combination indices (CI) according to the method of isoboles.

A composition described herein may comprise at least a first cannabinoid and a second cannabinoid. In some embodiments, the first cannabinoid is an acidic cannabinoid. In some embodiments, the first cannabinoid is cannabidivarinic acid, cannabigerovarinic acid, cannabidiolic acid, cannabigerolic acid, tetrahydrocannabivarinic acid, cannabinolic acid, tetrahydrocannabinolic acid, cannabichromenic acid, or cannabicyclolic acid. In some embodiments, the first cannabinoid is an acidic cannabinoid. In some embodiments, the first cannabinoid is cannabigerolic acid. In some embodiments, the second cannabinoid is an acidic cannabinoid. In some embodiments, the second cannabinoid is cannabidiolic acid (CBDA), cannabidivarin (CBDV), cannabigerol (CBG), cannabidiol (CBD), tetrahydrocannabinolic acid (THCA), cannabigerovarinic acid (CBGVA), or tetrahydrocannabivarinic acid (THCVA).

Compositions comprising two or more cannabinoids may be present in a variety of ratios. In some embodiments, a first cannabinoid is cannabigerolic acid (CBGA). In some embodiments, a second cannabinoid is cannabidivarin (CBDV). Such ratios are described in terms of mole ratio or in terms of a weight ratio. In some embodiments, the mole ratio of a first cannabinoid to a second cannabinoid is about 100:1, 50:1, 25:1, 20:1, 15:1, 10:1, 5:1, 2:1, or 1:1. In some embodiments, the mole ratio of a first cannabinoid to a second cannabinoid is at least 100:1, 50:1, 25:1, 20:1, 15:1, 10:1, 5:1, 2:1, or 1:1. In some embodiments, the mass ratio of a first cannabinoid to a second cannabinoid is about 100:1, 50:1, 25:1, 20:1, 15:1, 10:1, 5:1, 2:1, or 1:1. In some embodiments, the mass ratio of a first cannabinoid to a second cannabinoid is at least 100:1, 50:1, 25:1, 20:1, 15:1, 10:1, 5:1, 2:1, or 1:1. In some embodiments, the mass ratio of a first cannabinoid to a second cannabinoid is 100:1-1:1, 50:1-1:1, 25:1-1:1, 100:1-50:1, 50:1-10:1, 20:1-1:1, 10:1-1.5:1.

### Inhibitory Compounds

Compositions described herein may have one or more effects on cells. In some embodiments, the cells comprise immune cells. Immune cells in some instances comprise lymphocytes, monocytes, neutrophils, leukocytes, phagocytes, macrophages, microglia, mast cells, or other immune cells. Lymphocytes include but are not limited to T-cells, B-cells, NK-cells, helper T-cells, cytotoxic T lymphocytes. In some embodiments, the effect comprises an inhibitory effect on one or more cellular processes in such cells. In some embodiments, the cellular process comprises activation of one or more immune cells. Without being bound by theory, compositions described herein modulate calcium influx in immune cells. In some embodiments, modulation comprises inhibition of calcium channels. In some embodiments, inhibition of calcium influx comprises the mechanism of Store-Operated Calcium Entry. In some embodiments, the cellular process comprises secretion of cytokines or chemokines. In some embodiments, secretion of cytokines is inhibited in two or more immune cells. In some embodiments, the cytokines comprise those involved in inflammation. In some embodiments, the secretion of two or more cytokines is inhibited. Exemplary cytokines include but are not limited to interleukin-1 (IL-1), IL-12, and IL-18, tumor necrosis factor alpha (TNF-α), interferon gamma (IFNγ), and granulocyte-macrophage colony stimulating factor (GM-CSF).

An inhibitory effect may be measured by a percent inhibition relative to cells without treatment using the compositions described herein. In some embodiments, secretion of at least one cytokine (e.g., inflammatory cytokine) is reduced by about 5%, 10%, 20%, 30%, 50%, 75%, 100%, 200%, 500%, or about 1,000%. In some embodiments, secretion of at least one cytokine (e.g., inflammatory cytokine) is reduced by at least 5%, 10%, 20%, 30%, 50%, 75%, 100%, 200%, 500%, or at least 1,000%. In some embodiments, secretion of at least one cytokine (e.g., inflammatory cytokine) is reduced by 5-25%, 20-100%, 30-150%, 15-75%, 100-1,000%, 250-500%, or 500-1000%.

An inhibitory effect may be measured as a degree of inhibition, defined as the ratio of the rate in the absence of inhibitor vₒ vs. the rate in the presence of inhibitor vi. An inhibitory effect may be measured by half the concentration of drug needed to achieve inhibition of the target (IC₅₀). In some embodiments, the composition described herein inhibits release of a cytokine (e.g., inflammatory cytokine) with an IC₅₀ of about 50 μM, 25 μM, 10 μM, 5 μM, 1 μM, 500 nM, 250 nM, 200 nM, 150 nM, 100 nM, 50 nM, 25 nM, 10 nM, 5 nM, or about 1 nM. In some embodiments, the composition described herein inhibits release of a cytokine (e.g., inflammatory cytokine) with an IC₅₀ of no more than 50 μM, 25 μM, 10 μM, 5 μM, 1 μM, 500 nM, 250 nM, 200 nM, 150 nM, 100 nM, 50 nM, 25 nM, 10 nM, 5 nM, 1 nM or no more than 0.1 nM. In some embodiments, the composition described herein inhibits release of a cytokine (e.g., inflammatory cytokine) with an IC₅₀ of about 1-100 μM, 0.5-50 μM, 1-10 μM, 1-100 nM, 0.1-50 nM, 50-500 nM, 10-100 nM, 0.1-100 nM, 100-500 nM, or 0.1-10 nM.

### Pharmaceutical Composition/Formulation.

A pharmaceutical composition can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions in some instances are administered in therapeutically-effective amounts as pharmaceutical compositions by various forms and routes including, for example, intravenous, subcutaneous, intramuscular, oral, rectal, aerosol, parenteral, ophthalmic, pulmonary, transdermal, vaginal, otic, nasal, and topical administration. In some instances, a pharmaceutical composition comprises a cannabinoid and at least one excipient.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated readily by combining the active compounds with pharmaceutically-acceptable carriers or excipients. Such carriers can be used to formulate tablets, powders, pills, dragees, capsules, liquids, gels, syrups, elixirs, slurries, suspensions and the like, for oral ingestion by a subject.

Pharmaceutical preparations for oral use can be obtained by mixing one or more solid excipients with one or more of the compounds described herein, optionally grinding the resulting mixture, and processing the mixture of granules, after adding suitable auxiliaries, if desired, to obtain tablets or dragee cores. Cores in some instances are provided with suitable coatings. For this purpose, concentrated sugar solutions are in some instances used, which can contain an excipient such as gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline-processed. The push-fit capsules can contain the active ingredients in admixture with filler such as lactose, binders such as starches, and/or lubricants such as talc or magnesium stearate and, stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Parental injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

Formulations suitable for transdermal administration of the active compounds can employ transdermal delivery devices and transdermal delivery patches, and can be lipophilic emulsions or buffered aqueous solutions, dissolved and/or dispersed in a polymer or an adhesive. Such patches can be constructed for continuous, pulsatile, or on demand delivery of pharmaceutical compounds. Transdermal delivery can be accomplished by means of iontophoretic patches and the like. Additionally, transdermal patches can provide controlled delivery. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin.

For administration by inhalation, the active compounds can be in a form as an aerosol, a mist, or a powder. Pharmaceutical compositions are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compounds and a suitable powder base such as lactose or starch.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone and PEG. In suppository forms of the compositions, a low-melting wax such as a mixture of fatty acid glycerides or cocoa butter can be used.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising the compounds described herein can be manufactured, for example, by mixing, dissolving, granulating, dragee-making, levigating, emulsifying, encapsulating, entrapping, or compression processes.

The pharmaceutical compositions can include at least one pharmaceutically acceptable carrier, diluent, or excipient and compounds described herein as free-base or pharmaceutically-acceptable salt form. The methods and pharmaceutical compositions described herein include the use crystalline forms (also known as polymorphs), and active metabolites of these compounds having the same type of activity.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Non-limiting examples of pharmaceutically-acceptable excipients suitable for use include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

A composition can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, granular masses, and the like.

A controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Effective dosages may be determined from the blood or plasma concentration of drug. In some embodiments, the effective dosage for CBGA may be about 0.1 ng/mL to about 1000 ng/mL. In other instances, the effective dosage for CBGA may be about 0.5 ng/mL to about 1000 ng/mL. In still other instances, the effective dosage for CBGA may be about 1 ng/mL to about 900 ng/mL. In yet other instances, the effective dosage for CBGA may be about 5 ng/mL to about 700 ng/mL. In some instances, the effective dosage for CBGA may be about 10 ng/mL to about 500 ng/mL. In other instances, the effective dosage for CBGA may be about 15 ng/mL to about 400 ng/mL. In yet other instances, the effective dosage for CBGA may be about 20 ng/mL to about 300 ng/mL. In still other instances, the effective dosage for CBGA may be about 25 ng/mL to about 200 ng/mL. In some embodiments, the effective dosage for CBGA may be about 50 ng/mL to about 100 ng/mL.

In other instances, the effective dosage for CBGA may be at least about 0.1 ng/mL, at least about 0.5 ng/mL, at least about 1.0 ng/mL, at least about 2.5 ng/mL, at least about 5 ng/mL, at least about 10 ng/mL, at least about 25 ng/mL, at least about 50 ng/mL, at least about 100 ng/mL, at least about 250 ng/mL, at least about 500 ng/mL, at least about 750 ng/mL, at least about 900 ng/mL, at least about 950 ng/mL, at least about 990 ng/mL, or at least about 1000 ng/mL. In yet other instances, the effective dosage for CBGA may be not more than about 1000 ng/mL, not more than about 900 ng/mL, not more than about 800 ng/mL, not more than about 750 ng/mL, not more than about 700 ng/mL, not more than about 600 ng/mL, not more than about 500 ng/mL, not more than about 400 ng/mL, not more than about 300 ng/mL, not more than about 200 ng/mL, not more than about 100 ng/mL, not more than about 75 ng/mL, not more than about 50 ng/mL, not more than about 25 ng/mL, or not more than about 10 ng/mL.

In some embodiments, the effective dosage for the second cannabinoid may be about 0.1 ng/mL to about 1000 ng/mL. In other instances, the effective dosage for the second cannabinoid may be about 0.5 ng/mL to about 1000 ng/mL. In still other instances, the effective dosage for may be about 1 ng/mL to about 900 ng/mL. In yet other instances, the effective dosage for the second cannabinoid may be about 5 ng/mL to about 700 ng/mL. In some instances, the effective dosage for the second cannabinoid may be about 10 ng/mL to about 500 ng/mL. In other instances, the effective dosage for the second cannabinoid may be about 15 ng/mL to about 400 ng/mL. In yet other instances, the effective dosage for the second cannabinoid may be about 20 ng/mL to about 300 ng/mL. In still other instances, the effective dosage for the second cannabinoid may be about 25 ng/mL to about 200 ng/mL. In some embodiments, the effective dosage for the second cannabinoid may be about 50 ng/mL to about 100 ng/mL.

In other instances, the effective dosage for the second cannabinoid may be at least about 0.1 ng/mL, at least about 0.5 ng/mL, at least about 1.0 ng/mL, at least about 2.5 ng/mL, at least about 5 ng/mL, at least about 10 ng/mL, at least about 25 ng/mL, at least about 50 ng/mL, at least about 100 ng/mL, at least about 250 ng/mL, at least about 500 ng/mL, at least about 750 ng/mL, at least about 900 ng/mL, at least about 950 ng/mL, at least about 990 ng/mL, or at least about 1000 ng/mL. In yet other instances, the effective dosage for the second cannabinoid may be not more than about 1000 ng/mL, not more than about 900 ng/mL, not more than about 800 ng/mL, not more than about 750 ng/mL, not more than about 700 ng/mL, not more than about 600 ng/mL, not more than about 500 ng/mL, not more than about 400 ng/mL, not more than about 300 ng/mL, not more than about 200 ng/mL, not more than about 100 ng/mL, not more than about 75 ng/mL, not more than about 50 ng/mL, not more than about 25 ng/mL, or not more than about 10 ng/mL.

In still other instances, the effective dosage for CBGA for the treatment of an individual in need thereof, may be about 0.1 mg/kg to about 50 mg/kg body weight. In other instances, the effective dosage for CBGA may be about 0.01 mg/kg to about 500 mg/kg. In still other instances, the effective dosage for CBGA may be about 0.1 mg/kg to about 500 mg/kg. In yet other instances, the effective dosage for CBGA may be about 0.5 mg/kg to about 250 mg/kg. In some instances, the effective dosage for CBGA may be about 0.5 mg/kg to about 100 mg/kg. In other instances, the effective dosage for CBGA may be about 1 mg/kg to about 50 mg/kg. In yet other instances, the effective dosage for CBGA may be about 2.5 mg/kg to about 50 mg/kg. In still other instances, the effective dosage for CBGA may be about 5 mg/kg to about 40 mg/kg. In some embodiments, the effective dosage for CBGA may be about 1 mg/kg to about 25 mg/kg.

In other instances, the effective dosage for CBGA may be at least about 0.1 mg/kg body weight, at least about 0.5 mg/kg, at least about 1.0 mg/kg, at least about 2.5 mg/kg, at least about 5 mg/kg, at least about 10 mg/kg, at least about 25 mg/kg, at least about 50 mg/kg, at least about 100 mg/kg, at least about 250 mg/kg, or at least about 500 mg/kg. In yet other instances, the effective dosage for CBGA may be not more than about 500 mg/kg, not more than about 400 mg/kg, not more than about 300 mg/kg, not more than about 200 mg/kg, not more than about 100 mg/kg, not more than about 75 mg/kg, not more than about 50 mg/kg, not more than about 25 mg/kg, or not more than about 10 mg/kg.

In some embodiments, the effective dosage for a second cannabinoid in combination with CBGA for the treatment of an individual in need thereof may be about 0.01 mg/kg to about 500 mg/kg. In other instances, the effective dosage for a second cannabinoid. In still other instances, the effective dosage for a second cannabinoid may be about 0.1 mg/kg to about 500 mg/kg. In yet other instances, the effective dosage for a second cannabinoid may be about 0.5 mg/kg to about 250 mg/kg. In some instances, the effective dosage for a second cannabinoid may be about 0.5 mg/kg to about 100 mg/kg. In other instances, the effective dosage for a second cannabinoid may be about 1 mg/kg to about 50 mg/kg. In yet other instances, the effective dosage for a second cannabinoid may be about 2.5 mg/kg to about 50 mg/kg. In still other instances, the effective dosage for a second cannabinoid may be about 5 mg/kg to about 40 mg/kg. In some embodiments, the effective dosage for a second cannabinoid may be about 1 mg/kg to about 25 mg/kg.

In other instances, the effective dosage for a second cannabinoid in combination with CBGA for the treatment of an individual in need thereof may be at least about 0.1 mg/kg body weight, at least about 0.5 mg/kg, at least about 1.0 mg/kg, at least about 2.5 mg/kg, at least about 5 mg/kg, at least about 10 mg/kg, at least about 25 mg/kg, at least about 50 mg/kg, at least about 100 mg/kg, at least about 250 mg/kg, or at least about 500 mg/kg. In yet other instances, the effective dosage for a second cannabinoid in combination with CBGA may be not more than about 500 mg/kg, not more than about 400 mg/kg, not more than about 300 mg/kg, not more than about 200 mg/kg, not more than about 100 mg/kg, not more than about 75 mg/kg, not more than about 50 mg/kg, not more than about 25 mg/kg, or not more than about 10 mg/kg.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Methods of Administration and Treatment Methods.

Pharmaceutical compositions containing compounds described herein (e.g., cannabinoids of Table 1) can be administered for prophylactic and/or therapeutic treatments. In some embodiments, compositions described herein are used to treat inflammatory diseases. In some embodiments, compositions described herein are used to treat pain (acute or chronic). In some embodiments, the inflammatory pain relates to pain from skin, joints or GI tract disease or disorders. In therapeutic applications, the compositions can be administered to a subject already suffering from a disease or condition, in an amount sufficient to cure or at least partially arrest the symptoms of the disease or condition, or to cure, heal, improve, or ameliorate the condition. Compounds can also be administered to lessen a likelihood of developing, contracting, or worsening a condition. Amounts effective for this use can vary based on the severity and course of the disease or condition, previous therapy, the subject's health status, weight, and response to the drugs, and the judgment of the treating physician.

Treatment of Pain

Compositions described herein may be used for the treatment of pain. In some embodiments, pain is described by duration, such as acute or chronic pain. In some instances, acute pain is relatively short term, caused by a specific stimulus such as surgery, dental work, burns/lacerations, childbirth/labor, or broken bones. In some embodiments, chronic pain is defined as pain lasting at least a week, two weeks, a month, two months, three months, six months, nine months, a year, two years, or more than 5 years. In some embodiments, chronic pain is defined as pain lasting at least six months. In some embodiments chronic pain is manifested or caused by headaches, arthritis, cancer, nerve pain, back pain, or fibromyalgia. In some embodiments, chronic or acute pain is nociceptive, neurogenic, or psychogenic pain. In some embodiments, pain is described based on the underlying cause of the pain (e.g., disease, disorder, or trauma). In some embodiments, pain includes but is not limited to chronic pain, acute, nociceptive, breakthrough, soft tissue, visceral, somatic, phantom, cancer, inflammatory, or neuropathic pain. In some embodiments, pain is described relative to the area afflicted, such as head, skin, organs, muscles, tendons, spine, bone, or other part of the body.

Compositions described herein may be used to treat nociceptive pain. In some embodiments nociceptive pain includes but is not limited to radicular pain, somatic pain, or visceral pain. In some embodiments, radicular pain is caused by a radiculopathy, such as cervical, thoracic, or lumbar radiculopathy. In some embodiments, somatic pain is manifested by muscle pain, bone pain, skin pain, or headaches. In some embodiments, somatic pain is superficial (e.g., skin, mucus, and mucus membranes). In some embodiments, somatic pain is deep (tendons, joints, bones, muscles). In some embodiments, visceral pain is caused by inflammation. In some embodiments, somatogenic pain is muscular or skeletal (e.g., osteoarthritis, lumbosacral back pain, posttraumatic, myofascial), visceral (e.g., pancreatitis, ulcer, irritable bowel), ischemic (e.g., arteriosclerosis obliterans), or related to the progression of cancer (e.g., malignant or non-malignant).

Compositions described herein may be used to treat neurogenic pain. In some embodiments, neurogenic pain comprises neuropathic pain, central pain, or deafferentation pain. In some embodiments, neuropathic pain is caused by nerve damage or disease. In some embodiments, neuropathic pain comprises pain related to carpal tunnel syndrome, diabetic neuropathy, thalamic stroke and/or spinal cord injury. In some embodiments, central pain is caused by lesions of the central nervous system (e.g., thalamic pain). In some embodiments, deafferentation pain is caused by loss or interruption of sensory nerve fiber transmissions. In some embodiments, neurogenic pain is caused by posttraumatic and postoperative neuralgia. In some embodiments, neurogenic pain is caused by neuropathies (such as toxicity, or diabetes), causalgia, nerve entrapment, facial neuralgia, perineal neuralgia, postamputation, thalamic, or reflex sympathetic dystrophy.

Compositions described herein may be used to treat psychogenic pain. In some embodiments psychogenic pain results from psychological causes, such as mental, emotional, or behavioral factors. In some instances, psychogenic pain is manifested by headache, back pain, or stomach pain. In some instances, psychogenic pain is diagnosed by eliminating all other causes of pain.

Compositions described herein may be used to treat pain caused by specific disease, condition, disorder, or origin of pain. In some embodiments, compositions described herein are used to treat cancer pain (including metastatic or non-metastatic cancer), inflammatory disease pain, neuropathic pain, postoperative pain, iatrogenic pain (e.g., pain following invasive procedures or high dose radiation therapy, e.g., involving scar tissue formation resulting in a debilitating compromise of freedom of motion and substantial pain), complex regional pain syndromes, failed-back pain (e.g., acute or chronic back pain), soft tissue pain, joints and bone pain, central pain, injury (e.g., debilitating injuries, e.g., paraplegia, quadriplegia, etc., as well as non-debilitating injury (e.g., to back, neck, spine, joints, legs, arms, hands, feet, etc.)), arthritic pain (e.g., rheumatoid arthritis, osteoarthritis, arthritic symptoms of unknown etiology, etc.), hereditary disease (e.g., sickle cell anemia), infectious disease and resulting syndromes (e.g., Lyme disease, AIDS, etc.), headaches (e.g., migraine), causalgia, hyperesthesia, sympathetic dystrophy, phantom limb syndrome, denervation, and the like. In some embodiments, compositions described herein are used to treat pain associated with specific areas of the body, such the musculoskeletal system, visceral organs, head, bones, tendons, skin, nervous system, or other area of the body.

Treatment of Inflammatory Diseases

Compositions described herein may be used for the treatment of prevention of inflammatory diseases. In some embodiments, inflammatory diseases comprise diseases involving chronic inflammation. In some embodiments, such diseases include asthma, chronic peptic ulcer, tuberculosis, rheumatoid arthritis, periodontitis, ulcerative colitis, Crohn's disease, sinusitis, and active hepatitis. In some embodiments, such diseases include fibrosis, including Chronic Kidney Disease (CKD), renal fibrosis and other fibrotic diseases. In some embodiments, inflammatory diseases comprise autoimmune diseases. In some embodiments, an inflammatory disease includes but is not limited to Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (AIED); Autoimmune myocarditis; Autoimmune oophoritis; Autoimmune orchitis; Autoimmune pancreatitis; Autoimmune retinopathy; Autoimmune urticaria; Axonal & neuronal neuropathy (AMAN); Baló disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic Kidney Disease (CKD); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease; Congenital heart block; Coxsackie myocarditis; CREST syndrome; Crohn's disease; Dermatitis herpetiformis; Dermatomyositis; Devic's disease (neuromyelitis optica); Discoid lupus; Dressler's syndrome; Endometriosis; Eosinophilic esophagitis (EoE); Eosinophilic fasciitis; Erythema nodosum; Essential mixed cryoglobulinemia; Evans syndrome; Fibromyalgia; Fibrosis; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inversa); Hypogammalglobulinemia; IgA Nephropathy; IgG4-related sclerosing disease; Immune thrombocytopenic purpura (ITP); Inclusion body myositis (IBM); Interstitial cystitis (IC); Interstitial fibrosis (IF); Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus; Ligneous conjunctivitis; Linear IgA disease (LAD); Lupus; Lyme disease chronic; Meniere's disease; Microscopic polyangiitis (MPA); Mixed connective tissue disease (MCTD); Mooren's ulcer; Mucha-Habermann disease; Multifocal Motor Neuropathy (MMN) or MMNCB; Multiple sclerosis; Myasthenia gravis; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (PCD); Paroxysmal nocturnal hemoglobinuria (PNH); Parry Romberg syndrome; Pars planitis (peripheral uveitis); Parsonage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, III; Polymyalgia rheumatica; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PRCA); Pyoderma gangrenosum; Raynaud's phenomenon; Reactive Arthritis; Reflex sympathetic dystrophy; Relapsing polychondritis; Renal (kidney) fibrosis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Tolosa-Hunt syndrome (THS); Tubulointerstitial fibrosis; Transverse myelitis; Type 1 diabetes; Ulcerative colitis (UC); Undifferentiated connective tissue disease (UCTD); Uveitis; Vasculitis; Vitiligo; and Vogt-Koyanagi-Harada Disease.

Administration

Multiple therapeutic agents can be administered in any order or simultaneously. In some embodiments, a therapeutic agent comprises a composition described herein (e.g., comprising a cannabinoid of Table 1). If simultaneously, the multiple therapeutic agents can be provided in a single, unified form, or in multiple forms, for example, as multiple separate pills. The compounds can be packed together or separately, in a single package or in a plurality of packages. One or all of the therapeutic agents can be given in multiple doses. If not simultaneous, the timing between the multiple doses may vary to as much as about a month.

Compounds and compositions can be packaged as a kit. In some embodiments, a kit includes written instructions on the use of the compounds and compositions.

Compounds described herein can be administered before, during, or after the occurrence of a disease or condition, and the timing of administering the composition containing a compound can vary. For example, the compounds can be used as a prophylactic and can be administered continuously to subjects with a propensity to conditions or diseases in order to lessen a likelihood of the occurrence of the disease or condition. The compounds and compositions can be administered to a subject during or as soon as possible after the onset of the symptoms. The administration of the compounds can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A compound can be administered as soon as is practicable after the onset of a disease or condition is detected or suspected, and for a length of time necessary for the treatment of the disease, such as, for example, from about 1 month to about 3 months. The length of treatment can vary for each subject.

Dosage

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged tablets or capsules, and powders in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative. In some embodiments the pharmaceutical formulation unit dosage form is packaged into a container selected from the group consisting of a tube, ajar, a vial, a bag, a tray, a drum, a bottle, a syringe, a vape cartridge, and a can.

A compound described herein (e.g., CBGA and/or a second cannabinoid) can be present in a composition in a range of from about 1 mg to about 2500 mg; 1 mg to about 2000 mg; from about 5 mg to about 1000 mg, from about 5 mg to about 1200 mg, from about 10 mg to about 1000 mg, from about 25 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein (e.g., CBGA and/or a second cannabinoid) can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2100 mg, about 2200 mg, about 2300 mg, about 2400 mg or about 2500 mg.

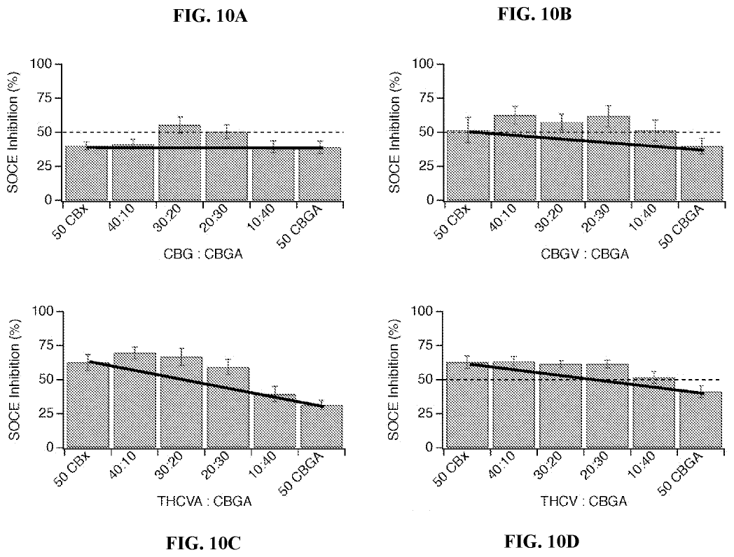

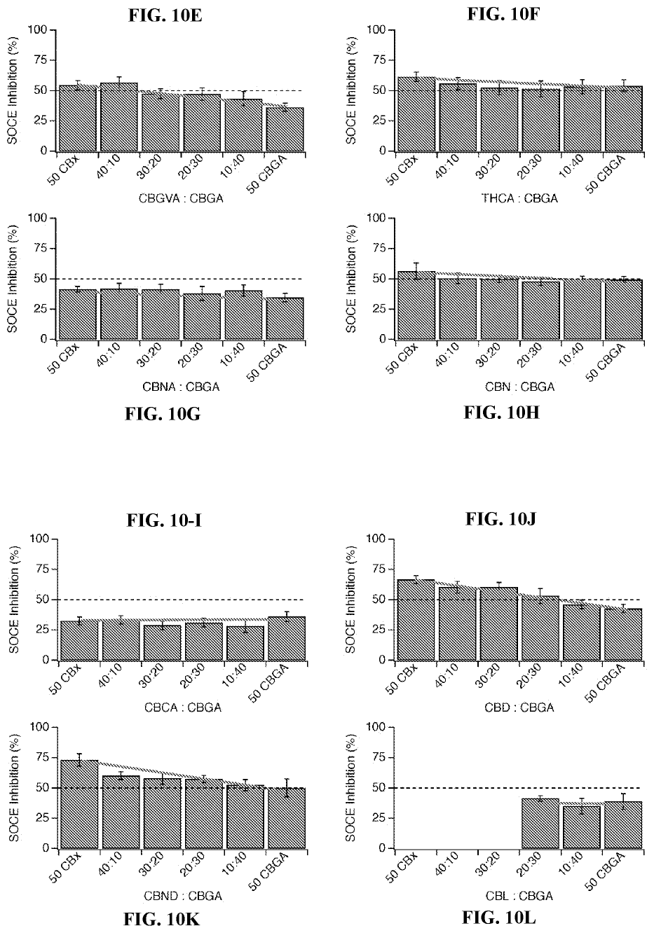

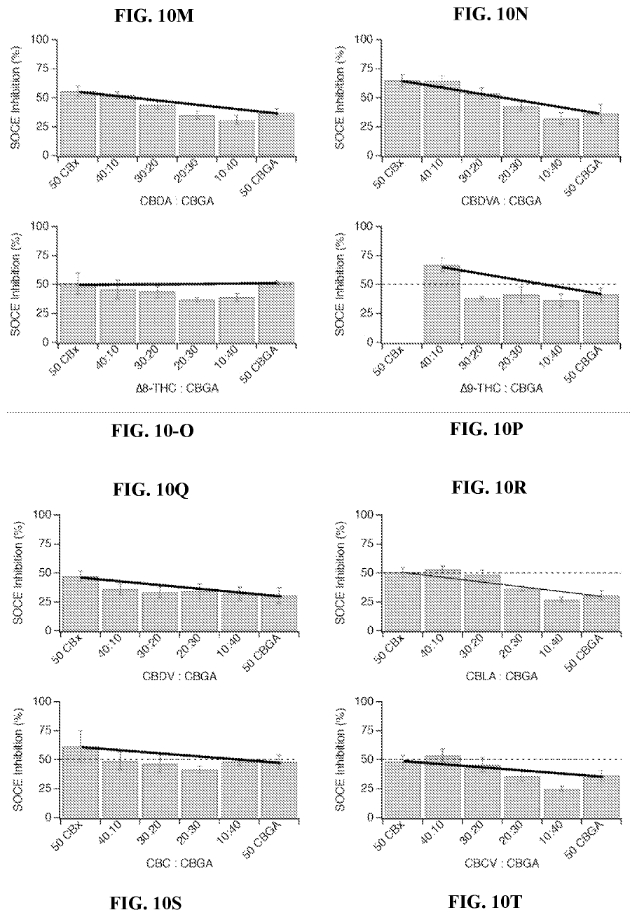

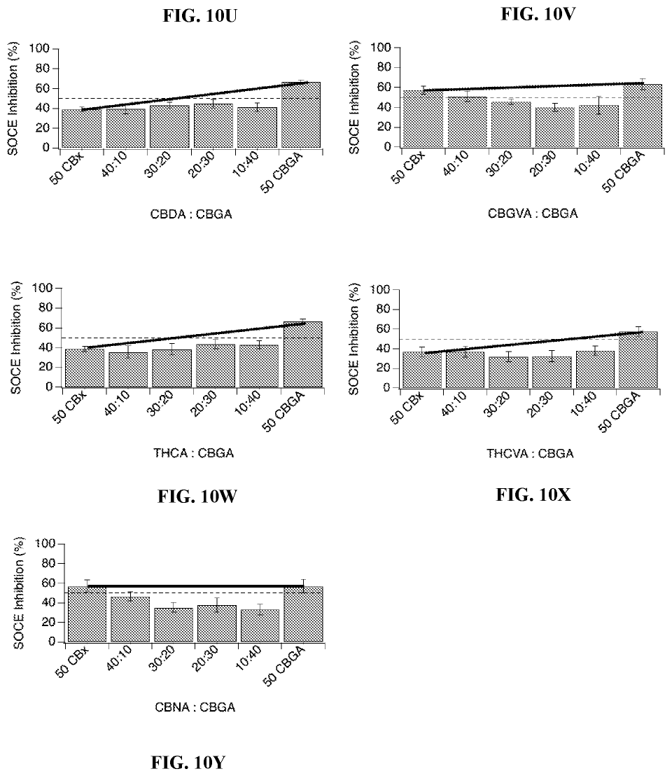

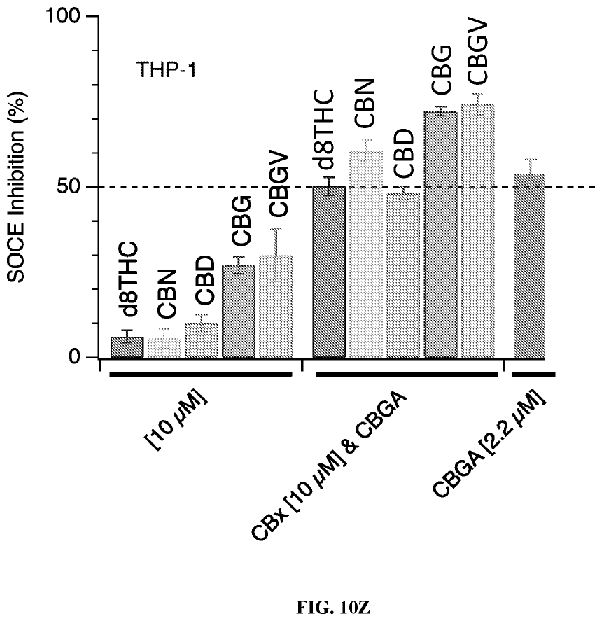

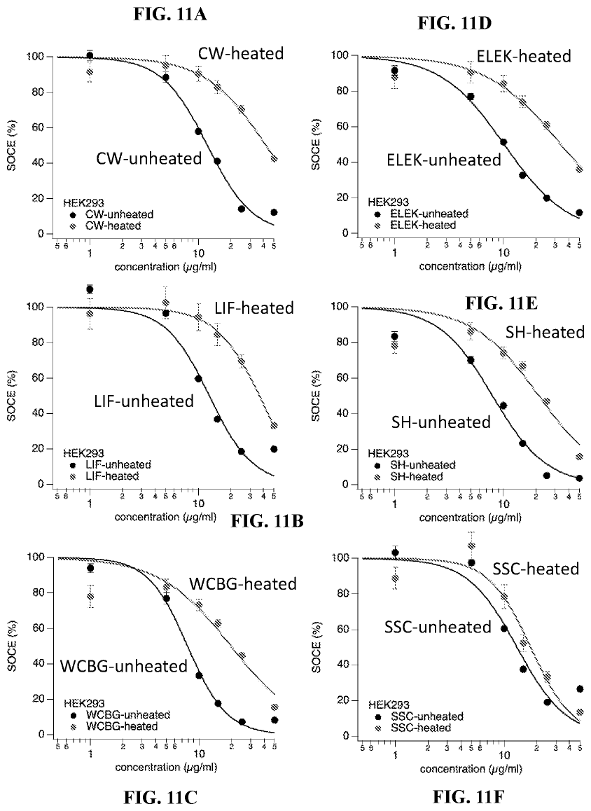

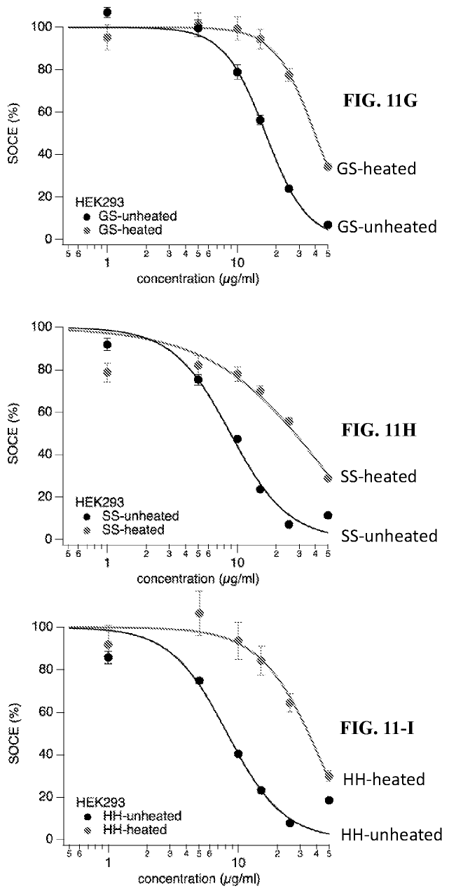

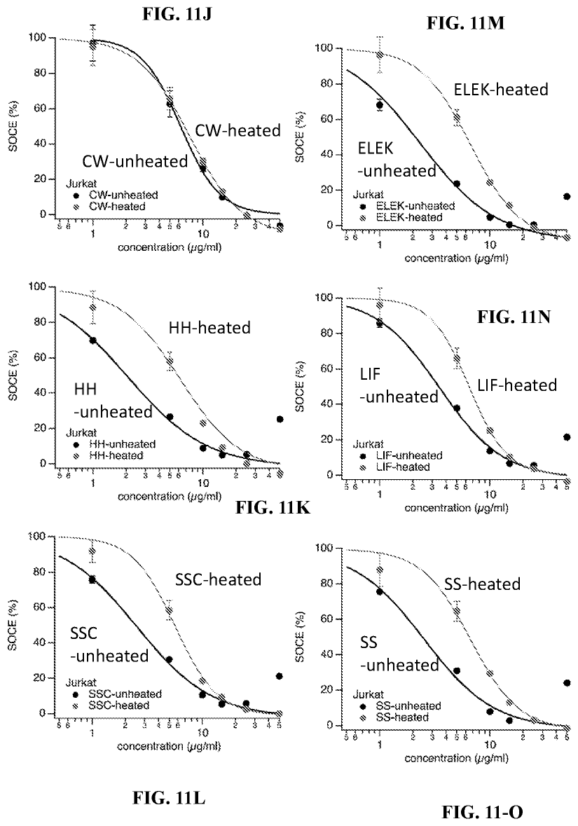

In some embodiments, a combination of CBGA and a second cannabinoid compound can be chosen depending upon the degree of activity, and the second cannabinoid compound provides a means for controlling the initial CBGA response in order to achieve a desired physiological or therapeutic activity or response. For example, in some instances, a synergistic activity can be obtained by combining, for example CBGA and CBG or CBD (see, e.g., FIG. 10A). In these instances, a large physiological or therapeutic activity may be desired, as seen when CBGA and CBG, for example, are added together. FIG. 10A. In other instances, potential side effects can be lessened, for example, if CBGA (and/or vice-versa with CBG) can be added in smaller amounts in order to achieve the same physiological or therapeutic effect. In other instances, a greater physiological or therapeutic response can be obtained when, for example, CBGA and CBG or CBD are used in combination, increasing the physiological or therapeutic effect as compared to the combined effect of CBGA, CBG or CBD when used alone.

In other embodiments, a second cannabinoid compound can be chosen in combination with CBGA, for example, to generate a sub-additive effect, for example, using a combination of CBGA and THCA or CBDA. See, e.g., FIG. 10A. In these instances, a smaller than expected physiological or therapeutic effect is seen when CBGA and THCA, for example, are added together. See FIG. 10A. In some instances, a sub-additive effect provides a means to control or fine-tune a physiological or therapeutic activity or response, for example, degree of calcium-release activated Ca²⁺ (CRAC) channel activation or inhibition, to predictably increase or decrease a desired physiological or therapeutic response. In other instances, use of CBGA with a second cannabinoid for example, THCA or CBDA, to generate a sub-additive effect can be used to decrease or lessen, for example, attendant side-effects.

In yet other instances, CBGA and at least a second cannabinoid compound, can be used to optimize a desired physiological or therapeutic response to a subject, including a human. In these instances, a supra-additive, additive, and/or subadditive combination of CBGA and at least a second cannabinoid compound can be used to obtain a desired or optimized degree of response or duration of action of the CBGA and at least second cannabinoid compound. In some instances, the desired or optimized degree of response or duration of action of the CBGA and at least a second cannabinoid compound is targeted to a specific tissue or organ system. See, e.g., Table 5, below. In some instances, the CBGA and at least a second cannabinoid compound targets, for example, cells, tissues or organ systems related to inflammatory conditions, cancer, pain, neurodegenerative conditions, autoimmune conditions and other diseases or conditions. In yet other instances, the CBGA and at least a second cannabinoid compound is chosen to optimize a degree of response and/or duration of action of the CBGA combination in a specific tissue or organ system, including, for example, cells, tissues or organ systems related to inflammatory conditions, cancer, pain, neurodegenerative conditions, autoimmune conditions and other diseases or conditions.

In still other instances, CBGA may be administered to an individual in need thereof to treat an inflammatory disorder or pain. In some instances, the inflammatory disorder may be fibrosis, including Chronic Kidney Disease (CKD), renal fibrosis, and other fibrotic diseases. In some instances, the effective dosage for CBGA may be about 0.1 ng/mL to about 1000 ng/mL. In other instances, the effective dosage for CBGA may be about 0.5 ng/mL to about 1000 ng/mL. In still other instances, the effective dosage for CBGA may be about 1 ng/mL to about 900 ng/mL. In yet other instances, the effective dosage for CBGA may be about 5 ng/mL to about 700 ng/mL. In some instances, the effective dosage for CBGA may be about 10 ng/mL to about 500 ng/mL. In other instances, the effective dosage for CBGA may be about 15 ng/mL to about 400 ng/mL. In yet other instances, the effective dosage for CBGA may be about 20 ng/mL to about 300 ng/mL. In still other instances, the effective dosage for CBGA may be about 25 ng/mL to about 200 ng/mL. In some embodiments, the effective dosage for CBGA may be about 50 ng/mL to about 100 ng/mL.

In other instances, the effective dosage for CBGA may be at least about 0.1 ng/mL, at least about 0.5 ng/mL, at least about 1.0 ng/mL, at least about 2.5 ng/mL, at least about 5 ng/mL, at least about 10 ng/mL, at least about 25 ng/mL, at least about 50 ng/mL, at least about 100 ng/mL, at least about 250 ng/mL, at least about 500 ng/mL, at least about 750 ng/mL, at least about 900 ng/mL, at least about 950 ng/mL, at least about 990 ng/mL, or at least about 1000 ng/mL. In yet other instances, the effective dosage for CBGA may be not more than about 1000 ng/mL, not more than about 900 ng/mL, not more than about 800 ng/mL, not more than about 750 ng/mL, not more than about 700 ng/mL, not more than about 600 ng/mL, not more than about 500 ng/mL, not more than about 400 ng/mL, not more than about 300 ng/mL, not more than about 200 ng/mL, not more than about 100 ng/mL, not more than about 75 ng/mL, not more than about 50 ng/mL, not more than about 25 ng/mL, or not more than about 10 ng/mL.

In some embodiments, the effective dosage for a second cannabinoid in combination with CBGA for the treatment of an individual in need thereof may be about 0.1 ng/mL to about 1000 ng/mL. In other instances, the effective dosage for second cannabinoid may be about 0.5 ng/mL to about 1000 ng/mL. In still other instances, the effective dosage for may be about 1 ng/mL to about 900 ng/mL. In yet other instances, the effective dosage for second cannabinoid may be about 5 ng/mL to about 700 ng/mL. In some instances, the effective dosage for second cannabinoid may be about 10 ng/mL to about 500 ng/mL. In other instances, the effective dosage for second cannabinoid may be about 15 ng/mL to about 400 ng/mL. In yet other instances, the effective dosage for second cannabinoid may be about 20 ng/mL to about 300 ng/mL. In still other instances, the effective dosage for second cannabinoid may be about 25 ng/mL to about 200 ng/mL. In some embodiments, the effective dosage for second cannabinoid may be about 50 ng/mL to about 100 ng/mL.

In other instances, the effective dosage for a second cannabinoid in combination with CBGA for the treatment of an individual in need thereof may be at least about 0.1 ng/mL, at least about 0.5 ng/mL, at least about 1.0 ng/mL, at least about 2.5 ng/mL, at least about 5 ng/mL, at least about 10 ng/mL, at least about 25 ng/mL, at least about 50 ng/mL, at least about 100 ng/mL, at least about 250 ng/mL, at least about 500 ng/mL, at least about 750 ng/mL, at least about 900 ng/mL, at least about 950 ng/mL, at least about 990 ng/mL, or at least about 1000 ng/mL. In yet other instances, the effective dosage for the second cannabinoid may be not more than about 1000 ng/mL, not more than about 900 ng/mL, not more than about 800 ng/mL, not more than about 750 ng/mL, not more than about 700 ng/mL, not more than about 600 ng/mL, not more than about 500 ng/mL, not more than about 400 ng/mL, not more than about 300 ng/mL, not more than about 200 ng/mL, not more than about 100 ng/mL, not more than about 75 ng/mL, not more than about 50 ng/mL, not more than about 25 ng/mL, or not more than about 10 ng/mL.

In still other instances, the effective dosage for CBGA for the treatment of fibrosis, including renal fibrosis, may be about 0.1 mg/kg to about 50 mg/kg body weight. In other instances, the effective dosage for CBGA may be about 0.01 mg/kg to about 500 mg/kg. In still other instances, the effective dosage for CBGA may be about 0.1 mg/kg to about 500 mg/kg. In yet other instances, the effective dosage for CBGA may be about 0.5 mg/kg to about 250 mg/kg. In some instances, the effective dosage for CBGA may be about 0.5 mg/kg to about 100 mg/kg. In other instances, the effective dosage for CBGA may be about 1 mg/kg to about 50 mg/kg. In yet other instances, the effective dosage for CBGA may be about 2.5 mg/kg to about 50 mg/kg. In still other instances, the effective dosage for CBGA may be about 5 mg/kg to about 40 mg/kg. In some embodiments, the effective dosage for CBGA may be about 1 mg/kg to about 25 mg/kg.

In other instances, the effective dosage for CBGA may be at least about 0.1 mg/kg body weight, at least about 0.5 mg/kg, at least about 1.0 mg/kg, at least about 2.5 mg/kg, at least about 5 mg/kg, at least about 10 mg/kg, at least about 25 mg/kg, at least about 50 mg/kg, at least about 100 mg/kg, at least about 250 mg/kg, or at least about 500 mg/kg. In yet other instances, the effective dosage for CBGA may be not more than about 500 mg/kg, not more than about 400 mg/kg, not more than about 300 mg/kg, not more than about 200 mg/kg, not more than about 100 mg/kg, not more than about 75 mg/kg, not more than about 50 mg/kg, not more than about 25 mg/kg, or not more than about 10 mg/kg.

In some embodiments, the effective dosage for a second cannabinoid in combination with CBGA for the treatment of an individual in need thereof may be about 0.01 mg/kg to about 500 mg/kg. In other instances, the effective dosage for a second cannabinoid. In still other instances, the effective dosage for a second cannabinoid may be about 0.1 mg/kg to about 500 mg/kg. In yet other instances, the effective dosage for a second cannabinoid may be about 0.5 mg/kg to about 250 mg/kg. In some instances, the effective dosage for a second cannabinoid may be about 0.5 mg/kg to about 100 mg/kg. In other instances, the effective dosage for a second cannabinoid may be about 1 mg/kg to about 50 mg/kg. In yet other instances, the effective dosage for a second cannabinoid may be about 2.5 mg/kg to about 50 mg/kg. In still other instances, the effective dosage for a second cannabinoid may be about 5 mg/kg to about 40 mg/kg. In some embodiments, the effective dosage for a second cannabinoid may be about 1 mg/kg to about 25 mg/kg.

In other instances, the effective dosage for a second cannabinoid in combination with CBGA for the treatment of an individual in need thereof may be at least about 0.1 mg/kg body weight, at least about 0.5 mg/kg, at least about 1.0 mg/kg, at least about 2.5 mg/kg, at least about 5 mg/kg, at least about 10 mg/kg, at least about 25 mg/kg, at least about 50 mg/kg, at least about 100 mg/kg, at least about 250 mg/kg, or at least about 500 mg/kg. In yet other instances, the effective dosage for a second cannabinoid in combination with CBGA may be not more than about 500 mg/kg, not more than about 400 mg/kg, not more than about 300 mg/kg, not more than about 200 mg/kg, not more than about 100 mg/kg, not more than about 75 mg/kg, not more than about 50 mg/kg, not more than about 25 mg/kg, or not more than about 10 mg/kg.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

### Example 1

### High-Throughput 96-Well Microfluorimetric Bioassays

Example 1 describes the mechanism and the identification of proteins underlying store-operated calcium entry (SOCE).

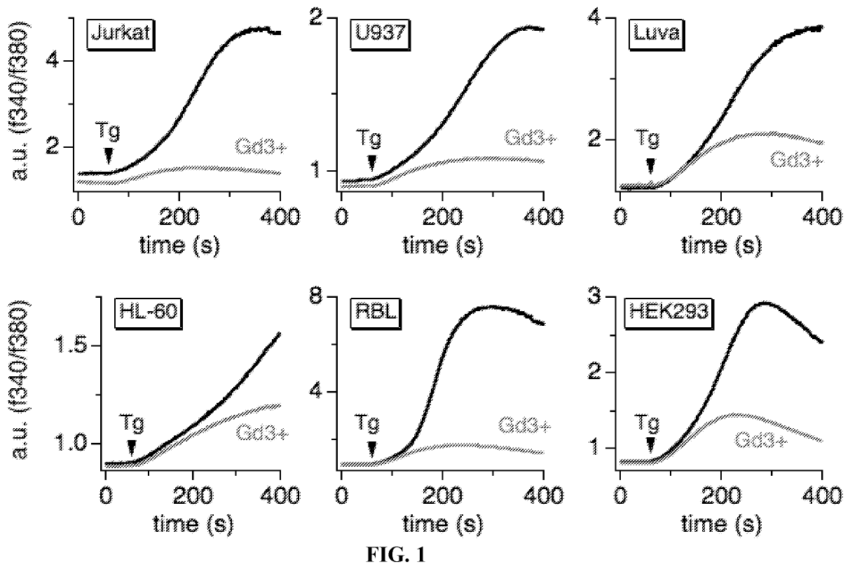

FIG. 1 shows examples of thapsigargin (Tg)-induced Ca²⁺ entry (SOCE) in a variety of immune cells (Jurkat T cells, U937 monocytes, Luva human mast cells, RBL-2H3 rat mast cells, and HL-60 neutrophils) and non-immune cells (HEK-293). A Fura-2 calcium flux assay was used to determine cytoplasmic Ca²⁺ levels. Briefly, cells were preloaded with Fura-2 AM, and fluorescence intensity in cells intensity was measured over time as a ratio of detected 510 nm fluorescent light emission intensity when excited by UV light at 340 nm and 380 nm wavelengths (f340/f380 ratio). Gadolinium (Gd³⁺) is known to block SOCE at a concentration of 1 μM and was used at 1 μM as a positive control in these experiments (gray line). As shown in FIG. 1, Tg treatment induced SOCE in all tested cell types as determined by f340/f380 ratio (black line), and Gd³⁺ inhibited SOCE in Tg-treated cells (gray line).

### Example 2

Intact population and single-cell fluorescent Ca²⁺ measurements with Fura-2-AM

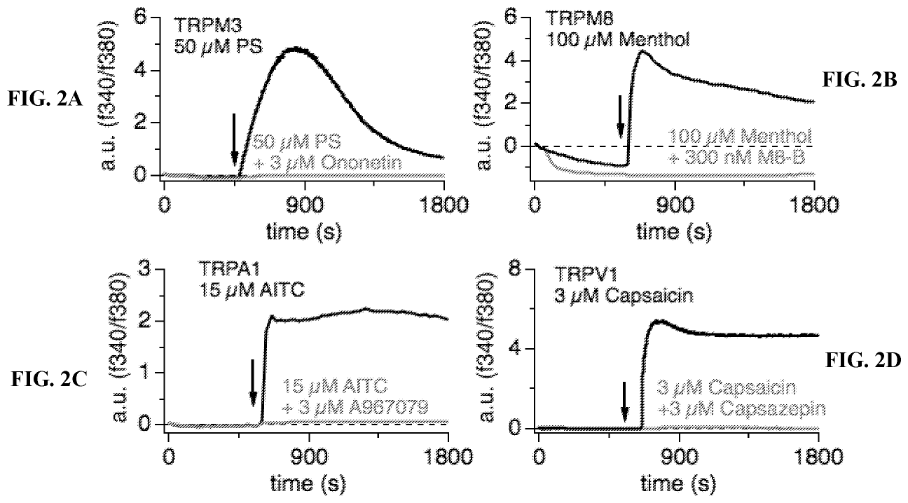

This example shows Fura-2 Ca²⁺ assays in 96- and 384-well high-throughput bioassay (HTS). HTS bioassays were developed to screen against four ion channels (TRPA1, TRPV1, TRPM3 and TRPM8) involved in pain sensation pathways (FIG. 2). These ion channels were overexpressed in tetracycline-inducible HEK293 cells, preloaded with Fura-2 AM as described in Example 1, and chemically stimulated to activate the overexpressed channels. HEK293 cells overexpressing TRPM3 were stimulated with 50 μM pregnenolone sulfate (PS) to activate TRPM3-mediated calcium mobilization (FIG. 2A, arrow; black line). HEK293 cells overexpressing TRPM3 channels were treated with 50 μM PS and 3 μM ononetin in control experiments to inhibit TRPM3-mediated calcium mobilization (FIG. 2A, arrow; gray line). HEK293 cells overexpressing TRPM8 were stimulated with 100 μM menthol to induce TRPM8-mediated calcium mobilization (FIG. 2B, arrow; black line). HEK293 cells overexpressing TRPM8 channels were treated with 100 μM menthol and 300 nM N-(2-aminoethyl)-N-[[3-methyoxy-4-(phenylmethoxy) phenyl]methyl]-2-thiophenecarboxamide, mono hydrochloride (M8-B) in control experiments to inhibit TRPM8-mediated calcium mobilization (FIG. 2B, arrow; gray line). HEK293 cells overexpressing TRPA1 were stimulated with 15 μM allyl isothiocyanate (AITC) to induce TRPA1-mediated calcium mobilization (FIG. 2C, arrow; black line). HEK293 cells overexpressing TRPA1 channels were treated with 15 μM AITC and 3 μM A967079 in control experiments to inhibit TRPA1-mediated calcium mobilization (FIG. 2C, arrow; gray line). HEK293 cells overexpressing TRPV1 were stimulated with 3 μM capsaicin to induce TRPV1-mediated calcium mobilization (FIG. 2D, arrow; black line). HEK293 cells overexpressing TRPV1 channels were treated with 3 μM capsaicin and 3 μM capsazepin in control experiments to inhibit TRPV1-mediated calcium mobilization (FIG. 2D, arrow; gray line).

FIG. 3 shows experimental data assessing agonist-induced Ca²⁺ oscillations, which can be a prerequisite for driving inflammatory cytokine release, in three individual human T lymphocytes (Jurkat cell line) using high-magnification Fura-2 fluorescence microscopy and digital image acquisition of single cells. Cytoplasmic calcium concentration oscillations were evoked by applying agonist phytohemagglutinin (PHA; 20 μg/ml), as shown in FIG. 3.

### Example 3

### Whole-Cell Patch Clamp Electrophysiology

This example shows experimental interrogation of calcium release mechanism in HEK293 immune cells using whole-cell patch clamping.

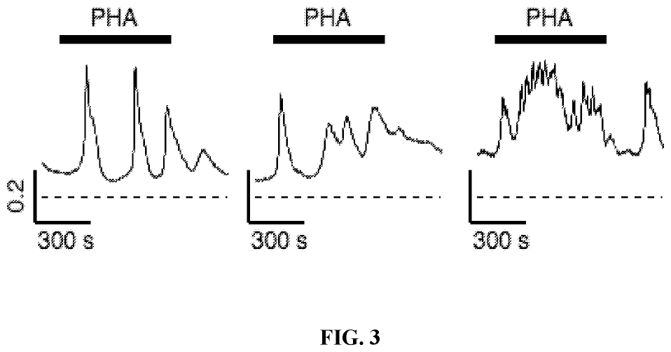

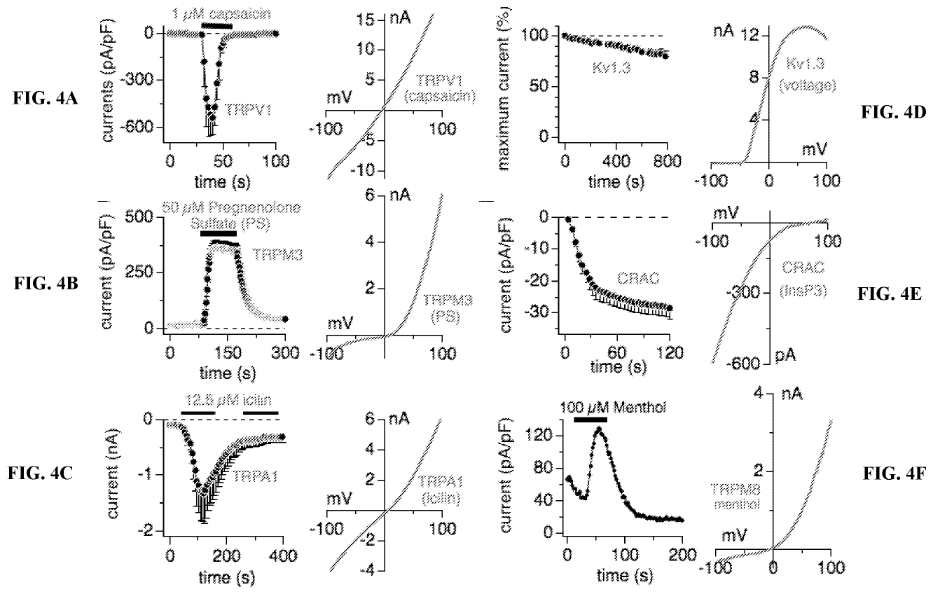

Whole-cell patch clamping was used to activate TRPV1 (FIG. 4A), TRPM3 (FIG. 4B), TRPA1 (FIG. 4C), Kv1.3 (FIG. 4D), I_{CRAC} (FIG. 4E) and TRPM8 (FIG. 4F) ion channels overexpressed in HEK293 cells. FIGS. 4A-4C and 4F: Left panels in each figure are averaged current development before, during and after agonist application (n=3-5, S.E.M.). Right panels are representative current-voltage traces extracted at the time of maximal current activation. Agonists used for assessing ion channel activity for TRPV1, TRPM3, TRPA1, Kv1.3, I_{CRAC}, and TRPM8 were 1 μM capsaicin, 50 μM pregnenolone sulfate (PS), 12.5 μM icilin, membrane depolarization, 50 μM inositol 1,4,5-trisphosphate (IP₃), and 100 μM menthol, respectively.

Voltage-gated potassium channel Kv1.3 was activated using a threshold voltage applied by the patch clamp pipette. I_{CRAC} channel activity was assessed using stimulation of inositol trisphosphate (IP₃) receptors with inositol trisphosphate. The left panels of FIGS. 4D and 4E show averaged current development of Kv1.3 currents by voltage activation (FIG. 4D) and CRAC currents (I_{CRAC}) by internal perfusion with 50 μM inositol 1,4,5-trisphosphate (IP₃) (FIG. 4E). Right panels are representative current-voltage traces extracted at the time of maximal current activation.

### Example 4

### Cytokine Release Assay

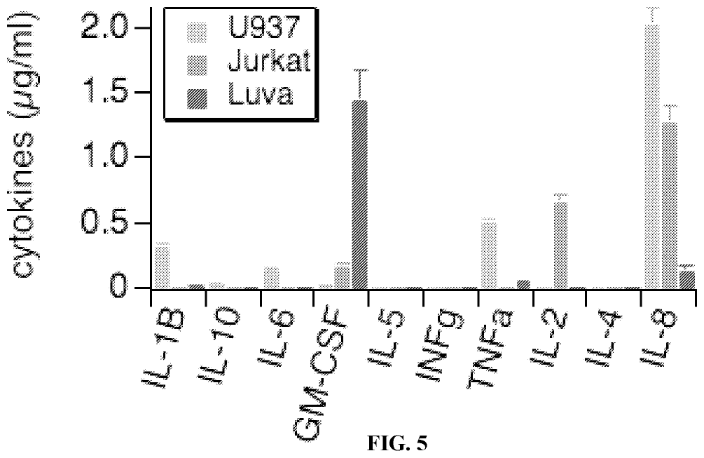

High-throughput multi-analyte bead-based immunoassay (Luminex® technology) combines a flow cytometer, fluorescently dyed microspheres (beads), lasers and digital signal processing to efficiently enable the detection and quantification of up to 100 targets within a single sample. Cytokine release in the immune cells of interest are shown in FIG. 5. The cytokine human 10-plex kit was used to simultaneously analyze a panel of the 10 most common pro-inflammatory cytokines (GM-CSF, IFNγ, IL-1β, IL-2, IL-4, IL-5, IL-6, IL-8, IL-10, TNF-α). In the assay, U937 cells, Jurkat cells, or Luva cells were seeded at a density of 2 million cells/ml and stimulated for 24 h. Supernatants were analyzed for cytokine content. Jurkat cells were stimulated with 50 ng/ml phorbol 12-myristate 13-acetate (PMA) and 1 μM ionomycin, U937 with 10 ng/ml PMA and 20 μg/ml lipopolysaccharide (LPS), Luva by 6 μM ionomycin. Results show that the respective agonists induce cytokine production in U937, Jurkat, and Luva cell lines.

### Example 5

### Analytical Chemistry: Extraction and Characterization of Phytochemicals

Plant material (Chemovar S04 obtained from NIDA) was subjected to a two-step extraction protocol using supercritical carbon dioxide to obtain terpene-rich (P≤1500 psi, T≤45° C.) and terpene-deficient (1500<P≤3500 psi, 45° C.<T≤60° C.) extracts. HPLC-DAD-MS analyses (HPLC-DAD is HPLC diode array detector analysis) of the extracts and commercial standards were used to identify known components (FIG. 6). FIG. 6 shows HPLC-UV (210 nm) traces of the terpene-deficient (TerpDefExt) and terpene-rich (TerpRichExt) extracts of the Cannabis plant material (NIDA Chemovar S04) and mixtures of commercial standards of terpenes and cannabinoids. Terpenes, flavonoids and lignans standards were obtained for use in these experiments. TerpMixA (terpene standards) comprised linalool (peak 2), β-myrcene (peak 13), terpinolene (peak 14), limonene (peak 18), α-pinene (peak 22). TerpMixB (terpene standards) comprised terpineol (peak 1), caryophyllene oxide (peak 8), ocimene (peak 12), γ-terpinene (peak 15), β-pinene (peak 19), A-carene (peak 21). TerpMixC (terpene standards): fenchol (no UV), camphene (peak 16), α-phellandrene (peak 17), α-humulene (peak 27), β-caryophyllene (peak 28). CB Std. (cannabinoid standards) comprised CBDVA (peak 3), CBND (peak 4), CBDV (peak 5), CBDA (peak 6), CBGA (peak 7), CBG (peak 9), CBD (peak 10), THCV (peak 11), CBN (peak 20), Δ⁹-THC (peak 23), Δ⁸-THC (peak 24), THCA (peak 25), CBC (peak 26). HPLC-DAD analysis showed that terpene-deficient extracts comprised CBDA, CBD and/or THCV, Δ⁹-THC, and THCA and/or CBC. Terpene-rich extracts were found to comprise β-myrcene (13), α-humulene (27), and β-caryophyllene (28).

### Example 6

### Cannabis Phytochemicals and Whole-Cannabis Plant Materials

Whole-plant Cannabis extracts, whole-plant dried Cannabis samples/specimens (chemovars) with various THC/CBD ratios, and individual cannabinoids were obtained from various suppliers, including NIDA, Cayman Chemical Company, Sigma-Aldrich. (Tables 2A, 2B and 3).

Table 2A shows percentages of various cannabinoids (by weight) in NIDA raw plant material samples.

| | NIDA Raw Plant Material | | | | | | | | | | |
|---|---|---|---|---|---|---|---|---|---|---|---|
| (weight %) | S01 | S02 | S03 | S04 | S05 | S06 | S07 | S08 | S09 | S10 | S11 |
| CBDVA | 0.0 | 0.0 | 0.0 | 0.0 | 0.1 | 0.1 | 0.1 | 0.0 | 0.0 | 0.0 | 0.0 |
| CBND | 0.0 | 0.0 | 0.0 | — | — | 0.0 | — | — | — | — | — |
| CBGVA | — | — | — | — | — | — | — | — | — | — | — |
| CBDV | 0.0 | 0.0 | 0.0 | 0.0 | 0.0 | 0.0 | 0.0 | 0.0 | 0.0 | 0.0 | 0.0 |
| CBDA | 0.0 | 0.0 | 2.2 | 3.9 | 11.1 | 6.6 | 7.5 | 3.1 | 2.8 | 0.0 | 2.7 |
| CBGA | 0.2 | 0.3 | 0.0 | 0.2 | 0.6 | 0.2 | 0.5 | 0.1 | 0.2 | 0.0 | 0.1 |
| CBG | 0.1 | 0.1 | 0.0 | 0.0 | 0.1 | 0.0 | 0.1 | 0.0 | 0.0 | 0.0 | 0.0 |
| CBD | 0.0 | 0.0 | 0.3 | 0.4 | 0.9 | 0.5 | 0.5 | 0.3 | 0.3 | 0.0 | 0.3 |
| THCV | 0.0 | 0.0 | 0.0 | 0.0 | 0.0 | 0.0 | 0.0 | 0.0 | 0.0 | 0.0 | 0.0 |
| CBCV | — | — | — | — | — | — | — | — | — | — | — |
| THCVA | 0.0 | 0.1 | 0.0 | 0.0 | 0.1 | 0.0 | 0.1 | 0.0 | 0.0 | 0.0 | 0.0 |
| CBN | 0.2 | 0.2 | 0.0 | 0.0 | 0.1 | 0.0 | 0.1 | 0.0 | 0.0 | 0.1 | 0.0 |
| CBNA | 0.3 | 0.4 | 0.0 | 0.1 | 0.2 | 0.0 | 0.2 | 0.1 | 0.1 | 0.1 | 0.0 |
| Δ9-THC | 0.7 | 1.0 | 0.0 | 0.4 | 0.7 | 0.0 | 0.7 | 0.2 | 0.2 | 0.1 | 0.1 |
| Δ8-THC | — | — | 0.0 | 0.0 | 0.0 | 0.0 | — | — | 0.0 | 0.0 | 0.0 |
| CBL | 0.0 | 0.1 | — | — | — | — | 0.1 | 0.0 | — | — | — |
| CBC | 0.0 | 0.0 | 0.0 | 0.0 | 0.0 | 0.0 | 0.0 | 0.0 | 0.0 | 0.0 | 0.0 |
| THCA | 6.3 | 9.6 | 0.1 | 1.9 | 5.7 | 0.2 | 6.5 | 1.6 | 1.4 | 1.0 | 0.6 |
| CBCA | 0.2 | 0.2 | 0.1 | 0.2 | 0.5 | 0.2 | 0.4 | 0.1 | 0.1 | 0.1 | 0.1 |
| CBLA | 0.0 | 0.0 | 0.0 | 0.0 | 0.0 | 0.0 | 0.0 | 0.0 | 0.0 | 0.0 | 0.0 |
| CBNM | — | — | — | — | — | — | — | — | — | — | — |

Table 2B shows percentages of various cannabinoids (by weight) in various hemp samples.

| | Hemp Samples | | | | | | | |
|---|---|---|---|---|---|---|---|---|
| | | | | White | Cherry | Suver | | |
| (weight %) | Otto18 | Har | Boax | CBG | Wine | Haze | Lifter | Elektra |
| CBDVA | 0.0 | 0.0 | 0.0 | 0.0 | 0.0 | 0.1 | 0.1 | 0.1 |
| CBND | 0.0 | — | 0.0 | — | 0.0 | — | — | 0.0 |
| CBGVA | — | — | — | — | — | — | — | — |
| CBDV | 0.0 | 0.0 | 0.0 | — | — | — | 0.0 | 0.0 |
| CBDA | 0.2 | 4.8 | 1.0 | 0.0 | 12.7 | 13.6 | 10.9 | 14.3 |
| CBGA | 0.0 | 0.2 | 0.0 | 14.4 | 0.2 | 0.3 | 0.3 | 0.3 |
| CBG | 0.0 | 0.0 | 0.0 | 0.6 | 0.0 | 0.0 | 0.0 | 0.1 |
| CBD | 0.4 | 0.8 | 0.1 | 0.0 | 1.7 | 0.7 | 0.9 | 1.2 |
| THCV | 0.0 | 0.0 | — | 0.0 | 0.0 | 0.0 | 0.0 | 0.0 |
| CBCV | 0.0 | — | — | — | — | — | — | — |
| THCVA | 0.0 | — | — | 0.0 | 0.0 | 0.0 | 0.0 | 0.0 |
| CBN | 0.0 | 0.0 | 0.0 | 0.0 | 0.0 | 0.0 | 0.0 | — |
| CBNA | 0.0 | 0.0 | — | 0.0 | — | 0.0 | 0.0 | — |
| Δ9-THC | 0.4 | 0.1 | 0.0 | 0.1 | 0.2 | 0.1 | 0.1 | 0.2 |
| Δ8-THC | — | 0.0 | 0.0 | 0.0 | — | 0.0 | 0.0 | 0.0 |
| CBL | 0.0 | 0.0 | — | 0.1 | 0.1 | 0.1 | 0.1 | 0.1 |
| CBC | 0.0 | 0.1 | 0.0 | 0.1 | 0.1 | 0.1 | 0.1 | 0.1 |
| THCA | 0.1 | 0.2 | 0.0 | 0.1 | 0.3 | 0.4 | 0.3 | 0.5 |
| CBCA | 0.0 | 0.5 | 0.1 | 0.3 | 0.5 | 0.9 | 0.5 | 0.7 |
| CBLA | 0.0 | 0.0 | 0.0 | — | — | 0.0 | 0.0 | 0.0 |
| CBNM | — | — | — | — | — | 0.0 | 0.0 | 0.0 |

| | Hemp Samples | | | | | | |
|---|---|---|---|---|---|---|---|
| | Sour | | | | | Plain | |
| | Space | Special | Hawaiian | Grape | | Jane | CBG |
| | Candy | Sauce | Haze | Soda | CBGenius | CBG | Bliss |
| CBDVA | 0.1 | 0.1 | 0.1 | 0.1 | 0.0 | 0.0 | 0.0 |
| CBND | 0.0 | — | — | — | — | — | — |
| CBGVA | — | — | — | — | — | — | — |
| CBDV | 0.0 | — | 0.0 | — | — | — | — |
| CBDA | 12.4 | 13.1 | 13.6 | 16.8 | 0.0 | 0.0 | 0.0 |
| CBGA | 0.2 | 0.7 | 0.3 | 0.3 | 7.6 | 15.8 | 8.9 |
| CBG | 0.1 | 0.1 | 0.0 | 0.1 | 0.3 | 0.8 | 1.1 |
| CBD | 1.6 | 0.5 | 0.5 | 1.2 | 0.0 | 0.0 | 0.0 |
| THCV | 0.0 | 0.0 | 0.0 | — | 0.0 | 0.0 | 0.0 |
| CBCV | 0.0 | — | — | — | — | — | 0.0 |
| THCVA | 0.0 | 0.0 | 0.0 | 0.0 | — | 0.0 | 0.0 |
| CBN | 0.0 | — | 0.0 | 0.0 | 0.0 | 0.0 | 0.0 |
| CBNA | 0.0 | — | 0.0 | 0.0 | — | — | 0.0 |
| Δ9-THC | 0.2 | 0.1 | 0.1 | 0.1 | 0.0 | 0.1 | 0.1 |
| Δ8-THC | 0.0 | 0.0 | 0.0 | 0.0 | — | 0.0 | 0.0 |
| CBL | 0.1 | 0.1 | 0.1 | 0.1 | 0.0 | 0.1 | 0.0 |
| CBC | 0.1 | 0.0 | 0.0 | 0.1 | 0.0 | 0.1 | 0.2 |
| THCA | 0.3 | 0.5 | 0.4 | 0.6 | 0.1 | 0.1 | 0.1 |
| CBCA | 0.5 | 0.8 | 0.6 | 0.7 | 0.1 | 0.4 | 0.2 |
| CBLA | 0.0 | 0.0 | 0.0 | 0.0 | — | 0.0 | — |
| CBNM | 0.0 | 0.0 | 0.0 | 0.0 | — | — | — |

Table 3 shows percentages of various cannabinoids (by weight percent (weight %)) in extracts obtained from NIDA.

| | NIDA Extracts | | |
|---|---|---|---|
| (weight %) | CBD | THC1 | THC2 |
| CBDVA | 0.0 | — | — |
| CBND | 0.0 | — | 0.2 |
| CBGVA | — | — | — |
| CBDV | 0.9 | 0.3 | 0.3 |
| CBDA | 0.2 | 0.1 | 0.1 |
| CBGA | — | — | 0.4 |
| CBG | 1.5 | 0.5 | 0.3 |
| CBD | 50.0 | 1.6 | 1.4 |
| THCV | 0.1 | 0.8 | 0.5 |
| CBCV | 0.0 | 0.0 | 0.0 |
| THCVA | 0.0 | — | — |
| CBN | 0.2 | 1.3 | 2.4 |
| CBNA | — | 0.1 | 0.1 |
| Δ9-THC | 2.0 | 29.6 | 19.3 |
| Δ8-THC | 0.0 | 0.0 | 0.0 |
| CBL | 0.1 | 0.3 | 0.3 |
| CBC | 2.5 | 2.3 | 2.0 |
| THCA | 0.0 | 0.2 | 0.1 |
| CBCA | — | 0.3 | 0.2 |
| CBLA | — | 0.1 | 0.1 |
| CBNM | — | 0.1 | — |

### Example 7

### Cannabinoid Inhibition of SOCE in HTS Bioassays

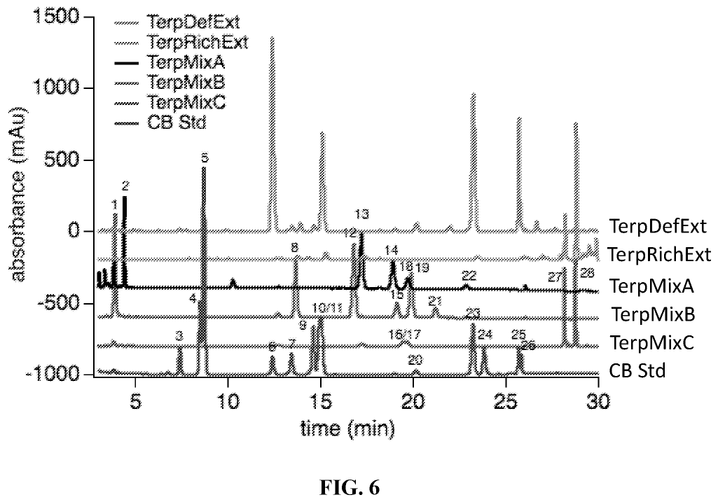

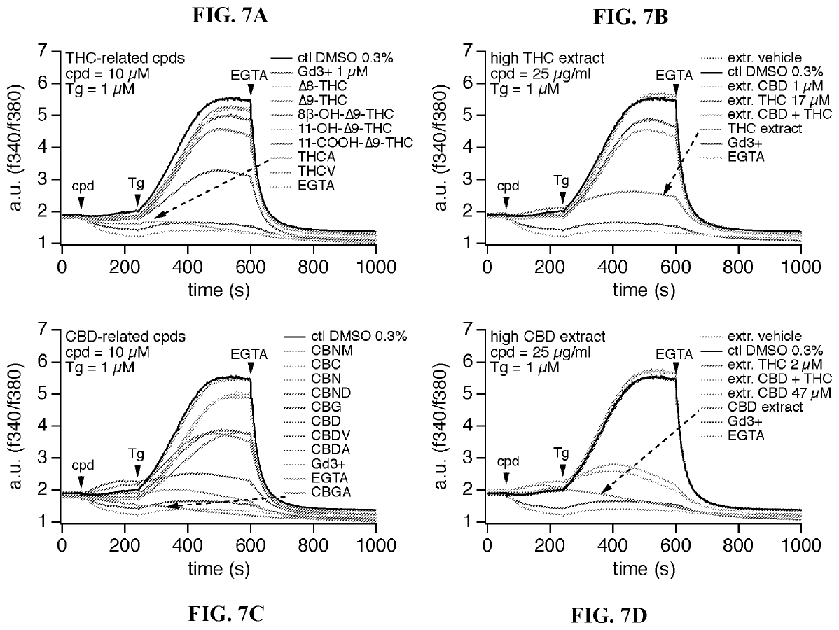

This example shows IC₅₀ values for various cannabinoids in NIDA raw plant material samples, extracts, and hemp in a SOCE bioassay. SOCE is the main Ca²⁺ entry mechanism and upstream signaling pathway in immune cell activation. Cannabis extracts and cannabinoids were screened against SOCE in Jurkat T cells (FIGS. 7A-7D). SOCE was experimentally solicited in intact Jurkat cells by applying 1 μM thapsigargin (Tg). Prior to this, the cells were pre-loaded with the Ca²⁺ sensitive dye Fura-2-AM (2 μM) for 1 hour. After washing the excess dye, cells were seeded in the assay plate (96-well plate) in physiological Ringer's solution containing 1 mM Ca²⁺. Fura-2 fluorescence emitted at 500 nm was then measured at excitation wavelengths of 340 and 380 nm. Emitted fluorescence intensities were processed by ratio analysis to obtain free intracellular Ca²⁺ concentrations [Ca²⁺]. After obtaining baseline levels for 60 s, Cannabis extracts or individual components were added to the individual wells of the assay plate and the resulting changes in [Ca²⁺]ᵢ were continuously monitored.

Table 4 shows IC₅₀ values for various cannabinoids in NIDA raw plant material samples, extracts, and hemp in a SOCE bioassay with various human cell lines.

| | IC50 (μg/mL) | Jurkat | U937 | Luva | RBL-2H3 | HEK293 |
|---|---|---|---|---|---|---|
| NIDA | S01 | 1.7 | 9.2 | 16 | 8.3 | 14 |
| Raw | S02 | 1.6 | 10 | 18 | 7.4 | 12 |
| Plant | S03 | 2.1 | 7.8 | 8.8 | 9.4 | 16 |
| Material | S04 | 1.9 | 9.2 | 9.2 | 7 | 10 |
| | S05 | 1.1 | 2.4 | 3.1 | 5.2 | 6 |
| | S06 | 1.5 | 5.8 | 7.4 | 5.9 | 8.8 |
| | S07 | 1.1 | 2 | 3.2 | 5.4 | 4.7 |
| | S08 | 1.9 | 9.1 | 10 | 8.2 | 10 |
| | S09 | 1.7 | 5.4 | 11 | 8.9 | 9.4 |
| | S10 | 5.9 | 22 | >50 | 22 | 18 |
| | S11 | 2.1 | 6.6 | 11 | 9.2 | 10 |
| NIDA | CBD | 5.1 | 24 | 12 | >50 | 33 |
| Extracts | THC1 | 9.8 | 37 | >50 | >50 | >50 |
| | THC2 | 9 | 35 | 42 | 44 | 39 |
| Hemp | Otto | 12 | 31 | 4.2 | >50 | >50 |
| | Har | 4.2 | 6.6 | 1.2 | 10 | 19 |
| | Boax | 22 | 37 | >50 | >50 | >50 |

### Example 8

### Individual and Combinatorial Cannabinoid Inhibition of SOCE in HTS Bioassays

This example shows IC₅₀ values for various cannabinoids in NIDA raw plant material samples, extracts, and hemp in an SOCE inhibition SOCE is the main Ca²⁺ entry mechanism and upstream signaling pathway in immune cell activation. Cannabis extracts and cannabinoids were screened against SOCE in Jurkat T cells (FIGS. 7A-7D). SOCE was experimentally solicited in intact Jurkat cells by applying 1 μM thapsigargin (Tg). Prior to this, the cells were pre-loaded with the Ca sensitive dye Fura-2-AM (2 μM) for 1 hour. After washing the excess dye, cells were seeded in the assay plate (96-well plate) in physiological Ringer's solution containing 1 mM Ca²⁺. Fura-2 fluorescence emitted at 500 nm was then measured at excitation wavelengths of 340 and 380 nm. Emitted fluorescence intensities were processed by ratio analysis to obtain free intracellular Ca²⁺ concentrations. After obtaining baseline levels for 60 s, Cannabis extracts or individual components were added to the individual wells of the assay plate and the resulting changes in [Ca²⁺]ᵢ were continuously monitored. Applying these compounds first allowed a determination of whether or not these compounds had any effect on calcium mobilization in unstimulated cells. At 240 s, thapsigargin was then applied to trigger the activation of SOCE. At the end of each assay, the calcium chelator, EGTA, was applied to confirm that the recorded signals are indeed a result of Ca²⁺ influx and to test for possible inhibition of the plasma membrane calcium ATPase (PMCA). All extracts were screened at 25 μg/ml, whereas the pure compounds were tested at 10 μM.

Table 5 illustrates IC₅₀ values in various human cell lines for purified cannabinoids described herein. Tested cell lines include Jurkat T lymphocytes cells harboring an NFAT-driven luciferase reporter cassette, U937 human monocytes, THP-1 human monocytes harboring an NT-kB response element-driven luciferase reporter cassette, Luva human mast cells, RBL-2H3 rat mast cells, and HEK-293 human embryonic kidney cells.

| IC50 μM | Jurkat | U937 | THP-1 | Luva | RBL-2H3 | HEK-293 |
|---|---|---|---|---|---|---|
| CBD | 7.42 | >50 | >50 | 2.53 | >50 | >50 |
| CBDA | 4.07 | 5.83 | 5.17 | 2.4 | 16.13 | 26.94 |
| CBG | 2.36 | 33.52 | 33.63 | 3.29 | >50 | >50 |
| CBGA | 0.53 | 2.23 | 2.21 | 0.27 | 5.72 | 3.6 |
| Δ9-THC | >50 | >50 | >50 | 0.97 | >50 | >50 |
| THCA | 2.23 | 8.1 | 10.39 | 0.57 | 17.53 | 20.7 |
| CBDV | 6 | 17.49 | >50 | 0.17 | 30.18 | 17.84 |
| CBDVA | 11.29 | 10.64 | 8.45 | 11.32 | 23.52 | 20.69 |
| CBGV | 5.27 | 14.51 | 40.7 | 6.46 | 30.85 | 9.16 |
| CBGVA | 1.15 | 2.86 | 2.6 | 2.53 | 4.15 | 2.11 |
| CBL | >50 | >50 | >50 | >50 | >50 | >50 |
| CBLA | 9.29 | 28.32 | 12.37 | 20.85 | >50 | 31.09 |
| CBNA | 0.99 | 19.18 | 7.25 | >50 | 22.49 | 29.46 |
| CBCA | 1.13 | >50 | 29.13 | >50 | >50 | >50 |
| THCVA | 0.633 | 20.16 | 9.43 | 20.21 | 21.34 | 35.8 |
| CBCV | 10.6 | >50 | >50 | 36.06 | 49.11 | 41.77 |
| CBND | 6.7 | 24 | >50 | 19 | 32.8 | 21.4 |
| THCV | 10.73 | >50 | >50 | >50 | >50 | >50 |
| CBN | 9.09 | 45.3 | >50 | 33.4 | 42.6 | 26.9 |
| Δ8-THC | 14.18 | >50 | >50 | 39.7 | >50 | >50 |
| CBC | 13.06 | 40.8 | >50 | 26.8 | 31.6 | 18.7 |
| CBNM | >50 | >50 | >50 | >50 | >50 | >50 |

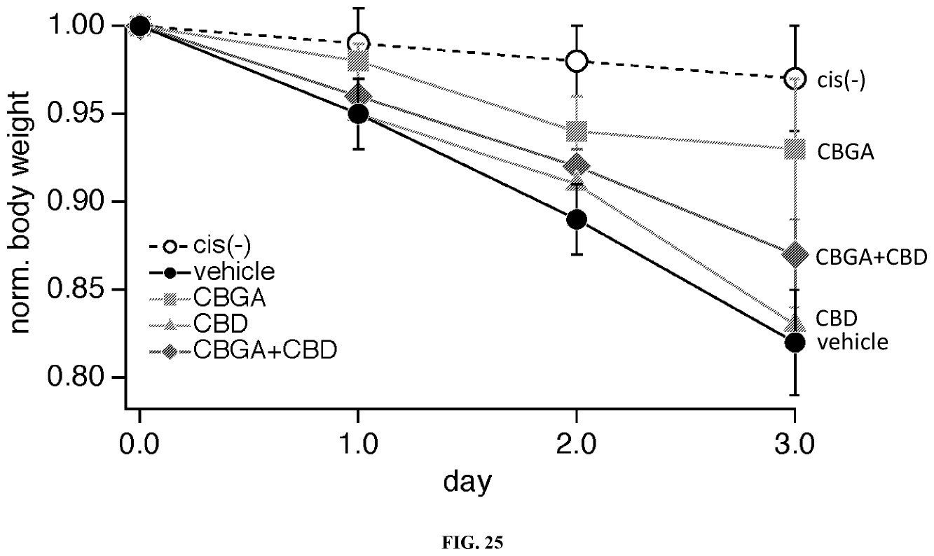

FIGS. 7A-7D show effects of cannabinoids on SOCE in Jurkat cells. Calcium signals are solicited in intact cells by applying 1 μM thapsigargin (Tg). Gadolinium (1 μM) is used here as a positive control (pos ctl) of SOCE inhibition. Seven THC derivatives (FIG. 7A), one high-THC extract (THC1 extract from NIDA) (FIG. 7B), nine CBD derivatives (FIG. 7C) and one high-CBD extract (CBD extract from NIDA) (FIG. 7D) were screened. The compounds and extracts were tested at 10 μM (FIGS. 7A and 7C) and 25 μg/ml (FIGS. 7B and 7D), respectively. To address the question whether the major cannabinoids CBD or Δ9-THC or a combination of both might account for the inhibitory effects of the extract, the equivalent concentrations of pure CBD, Δ9-THC, and their combination were tested based on their concentrations as derived from HPLC analysis. In the high-THC extract at 25 μg/ml, Δ9-THC was present at 17 μM and CBD at 1 μM (FIG. 7B) and in the high-CBD extract, CBD was present at 47 μM and Δ9-THC at 2 μM (FIG. 7D). All data are averages of 3 independent runs.

While CBD and THC show low to no effects at 10 μM, other cannabinoids (e.g., THCA, CBDA, CBGA) were able to fully block the Ca²⁺ signals, with potency similar to that of gadolinium (a known SOCE inhibitor, used here as a positive control) (FIG. 7A, FIG. 7C). High-THC extract inhibited SOCE (FIG. 7B, indicated by dotted black line). The high-CBD extract, on the other hand, showed full block of SOCE, an inhibition that may be due to its high CBD content of 47 μM (FIG. 7D, indicated by dotted black line).

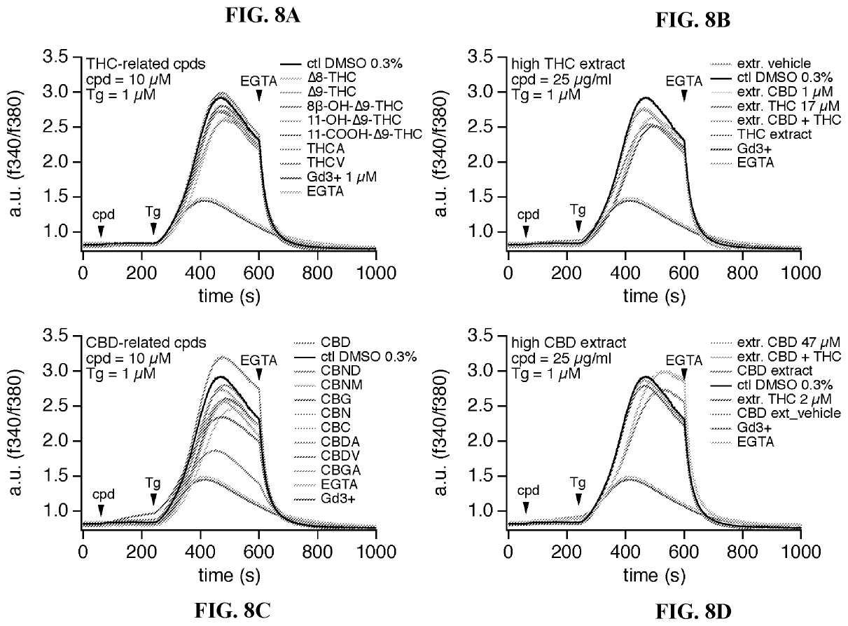

A screening of a non-immune cell line, HEK293 shown in FIGS. 8A-D, demonstrates that the inhibitory effects of the tested cannabinoids and extracts may be selective (e.g., 11-COOH-Δ9-THC, THCV, CBDV, CBDA) or more potent (e.g., CBGA) in immune cells. Without being bound by theory, these effects may be reflective of the difference in the expression profile of various Ca²⁺ players involved in immune cells vs. the ones involved in fibroblast HEK293 cells.

FIG. 8. shows effects of cannabinoids on SOCE in HEK293 cells. Calcium signals were solicited in intact cells by applying 1 μM thapsigargin (Tg). Gadolinium (1 μM) was used as a positive control (pos ctl) of SOCE inhibition. Seven THC derivatives were screened (FIG. 8A), a high-THC extract (FIG. 8B), nine CBD derivatives (FIG. 8C) and a high-CBD extract (FIG. 8D). The compounds and extracts were tested at 10 μM (FIGS. 8A and 8C) and 25 μg/ml (FIGS. 8B and 8D), respectively. All data are averages of 3 independent runs.

### Example 9

### Concentration-Response Effect of Cannabinoids

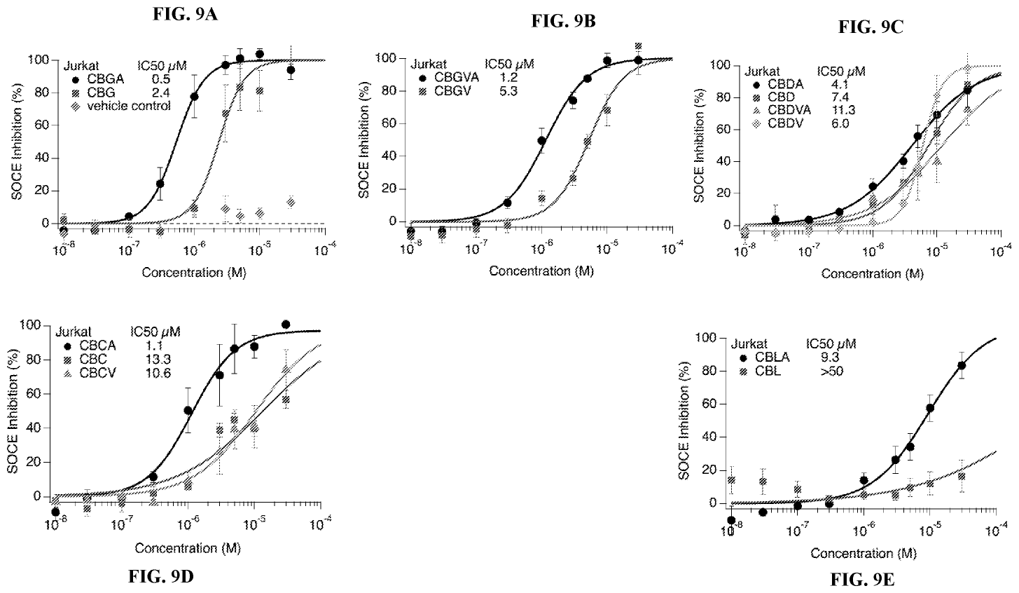

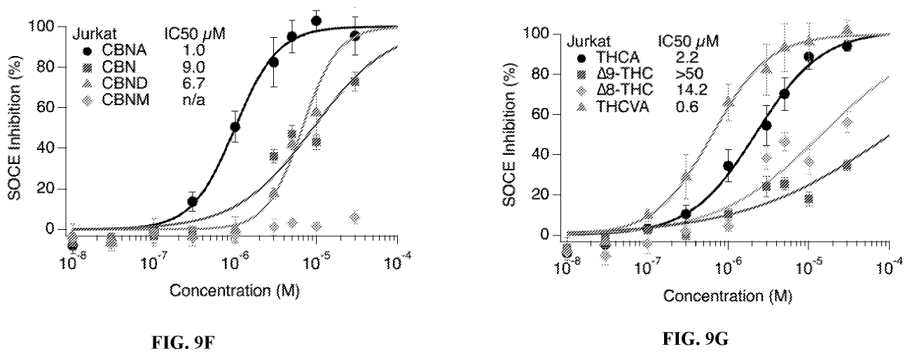

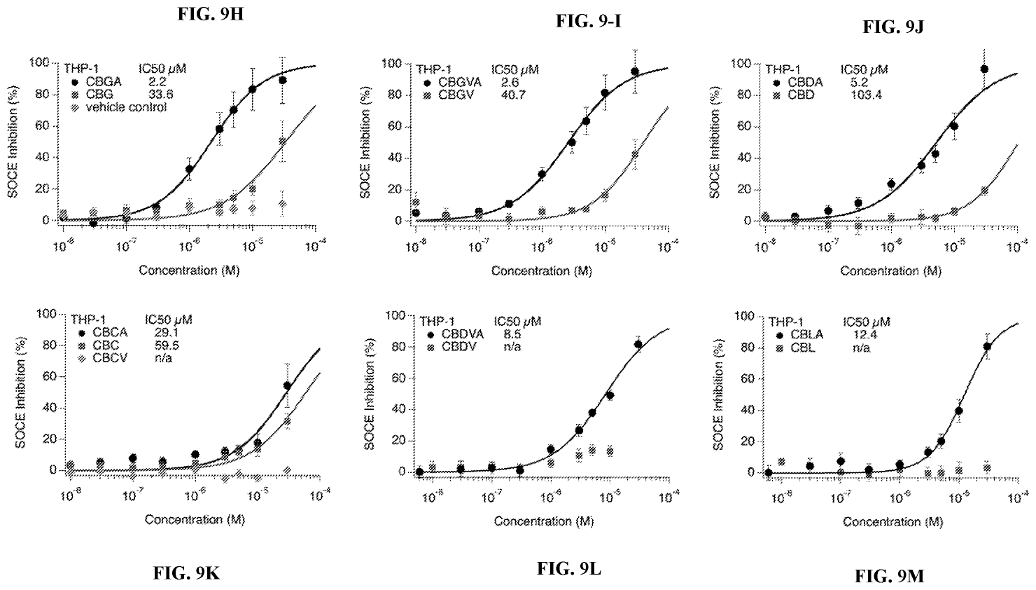

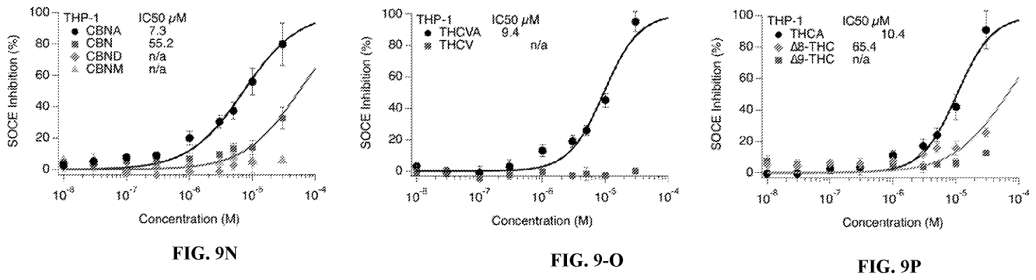

Cannabinoids that showed an inhibition rate of 40% or higher, (e.g., in Jurkat cells, as described in Example 8 and as shown in FIGS. 7A-7D), along with CBD and THC compounds, were screened for their dose-response behavior in SOCE assays performed as described in Example 8. The dose response experiments were carried out in Jurkat cells (FIGS. 9A-9G) and in THP-1 cells (FIGS. 9H-9P). Cannabinoids were tested at 9 different doses from 0 to 30 μM. SOCE inhibition was graphed as a percentage against molar concentration of the compounds assayed (FIGS. 9A-9P). IC₅₀ values were calculated based on dose response data and are shown in the inset legends of FIGS. 9A-9P and in Table 5. All data are averages of 3 independent runs±SEM. The effects on SOCE response (as calculated by area under the curve) were plotted in a dose-dependent manner to determine the IC₅₀ for each tested compound. FIGS. 9A-9F show compounds that are CBD derivatives tested in Jurkat cells. FIG. 9G shows compounds that are THC derivatives tested in Jurkat cells. FIGS. 9H-9N show compounds that are CBD derivatives tested in THP-1 cells. FIG. 9-O-FIG. 9P show compounds that are THC derivatives tested in THP-1 cells. FIGS. 9A-9P and Table 5 show cannabinoids like CBD, CBG, and THC have a higher IC₅₀ than their acidic variants.

### Example 10

### Combinatory Effects of Cannabinoids

This example shows analysis of combinatory effect of cannabinoids on store-operated calcium entry (SOCE) in human cells.

Part of the complexities of Cannabis use, as well as the large variability in outcomes, are linked to a combinatory effect, which is also known as entourage effect or ensemble effect. The existence of various chemovars of Cannabis as well as the multiple modes of use (e.g., edibles, vaporization, etc.) will affect the amounts and compositions of what is administered to the patients and, hence, the outcomes and degrees of efficacy for a given indication. In some cases, combinatory effect is beneficial. In some cases, combinatory effect is undesired. A prominent example of an undesired combinatory effect is in the use of THC for the treatment of glaucoma by lowering intraocular pressure. Without being bound by theory, the presence of CBD in a treatment comprising THC may antagonize THC effects, in some cases.

Combinatory effect studies using the most potent hit (e.g., candidate) from initial screening against Ca²⁺ signaling, namely CBGA (for example, as shown in Examples 8 and 9), in combination with other cannabinoids was conducted (FIGS. 10A-10BB). Bar graphs shown in FIGS. 10A-10Z and 10BB display SOCE amplitudes calculated from area under the curve (AUC) of the SOCE signals and normalized to SOCE in the presence of gadolinium. Combinatory effects were assessed using an isobolographic analysis approach, where CBGA was paired with another cannabinoid using concentrations of CBGA corresponding to 50%, 40%, 30%, 20%, 10%, and 0% inhibition (as derived from the IC₅₀ curves of FIGS. 9A-9P) and combined with concentrations of the paired cannabinoid, so that each combination would be expected to inhibit 50% of the Ca²⁺ signal (e.g., the CBGA concentration causing 50% inhibition was tested alone without the other cannabinoid, the CBGA concentration expected to block 40% of the signal was combined with the concentration of the other cannabinoid expected to inhibit 10%, the CBGA concentration expected to block 30% of the signal was combined with the concentration of the other cannabinoid expected to inhibit 20%, and so on). Simple additivity of cannabinoid pairs would be expected to inhibit the Ca²⁺ signal by 50%. All the data in these experiments represent an average of 3 independent runs and the values are graphed as mean values±SEM.

FIGS. 10A-10Y illustrate the inhibition of SOCE by paired cannabinoids comprising CBGA (e.g., via combinatory effect). FIGS. 10A-10T show inhibition of SOCE by individual or paired cannabinoids in Jurkat cells. FIGS. 10U-10Y show inhibition of SOCE by individual or paired cannabinoids in THP-1 cells. Straight dotted black lines drawn across bars of each graph illustrate the expected block at 50% if the compounds acted in a simple additive manner. Simple additive behavior was observed in blends of CBGA and each of: CBGVA, THCA, CBCA, CBD, CBNA, CBN, CBND, and CBL (see, e.g., FIGS. 10E-10L). In some cases (e.g., as shown in FIGS. 10A-10D), combinations of CBGA and another cannabinoid compound (e.g., CBG, CBGV, THCVA, or THCV) showed stronger than predicted inhibitions (e.g., supra-additive combinatory effect) of store-operated calcium entry. In some cases (e.g., as shown in FIGS. 10M-10T) show cannabinoids (e.g., selected from CBDA, CBDVA, CBDV, CBLA, Δ8-THC, and CBCV) that inhibit less than predicted when paired with CBGA in SOCE inhibition experiments performed in Jurkat cells (e.g., sub-additive combinatory effects). Combinations of CBGA and another cannabinoid (e.g., selected from CBDA, CBGVA, THCA, THCVA, and CBNA) tested in THP-1 cells showed sub-additive combinatory effects (see, FIGS. 10U-10Y).

Additional isobolographic analysis of combinatory effects of cannabinoids having SOCE IC₅₀ values greater than 30 μM were performed in THP-1 cells. In these experiments, candidate cannabinoid extracts (selected from Δ8-THC, CBN, CBD, CBG, and CBGV) were administered at 10 μM alone or in combination with 2.2 μM CBGA, which is a concentration near the SOCE IC₅₀ value of CBGA (see FIG. 10Z). The combination of 2.2 μM CBGA and 10 μM of any of CBN, CBG, and CBGV resulted in SOCE inhibition values greater than 50%, indicating that CBN, CBG, and CBGV are simple additive agonists, e.g., when paired with CBGA. Experimental results showed that Δ8-THC and CBD did not increase SOCE inhibition past 50% when paired with 2.2 μM CBGA, indicating that Δ8-THC and CBD are sub-additive (e.g., inhibitory), for example, when paired with 2.2 μM CBGA.

A partial agonist or antagonistic (e.g., sub-additive) effect by a first cannabinoid (e.g., CBDA) may be important in reducing or otherwise modulating side effects that may result from treatment with a second cannabinoid (e.g., CBGA), for example, which may be co-administered with the first cannabinoid. In addition, the ability of other cannabinoids to modulate the physiological or therapeutic activity of, for example, CBGA, may be useful in optimizing an appropriate level or degree of therapeutic response when needed.

Effects observed in these experiments may suggest chemical structure-activity relationships and some selectivity over non-immune cells. In addition, combined administration of minor and major cannabinoids support potential combinatory effects. While high-THC and high-CBD Cannabis extracts were active against SOCE in immune cells in these experiments, their activity was fully or partially carried out by components that are not THC or CBD.

### Example 11

### Effects on SOCE Inhibition by Heated and Unheated Extracts

This example shows effects of temperature on store-operated calcium entry (SOCE) in various cell types.

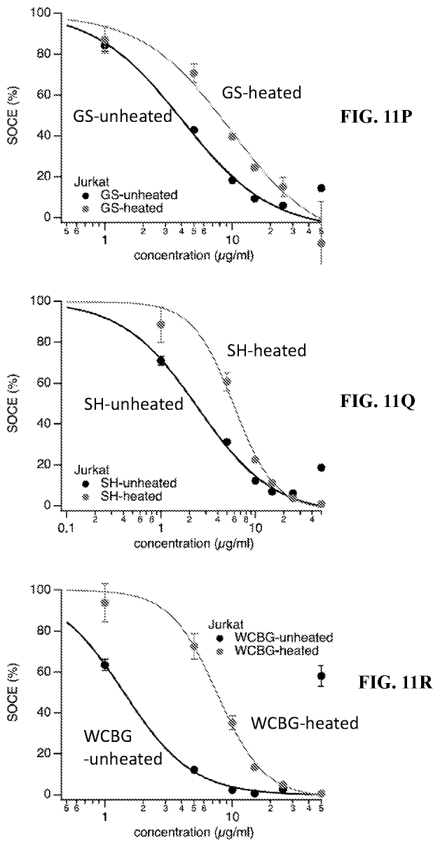

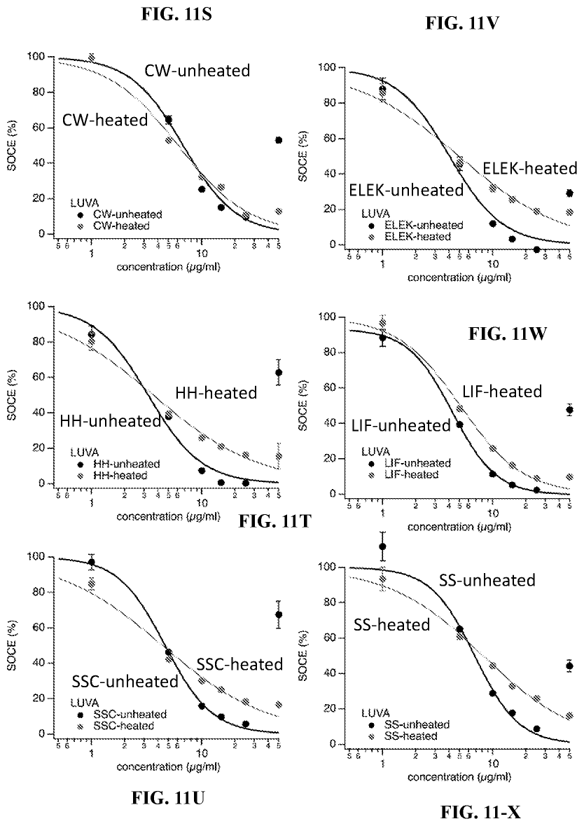

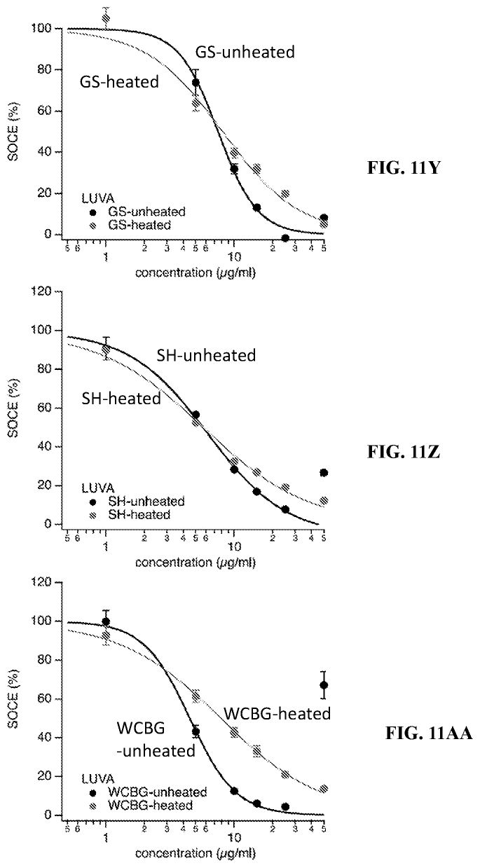

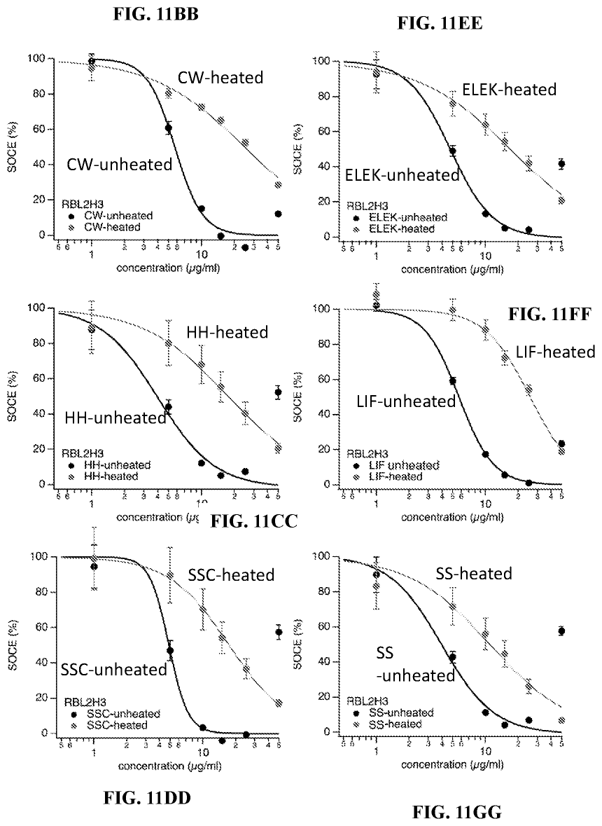

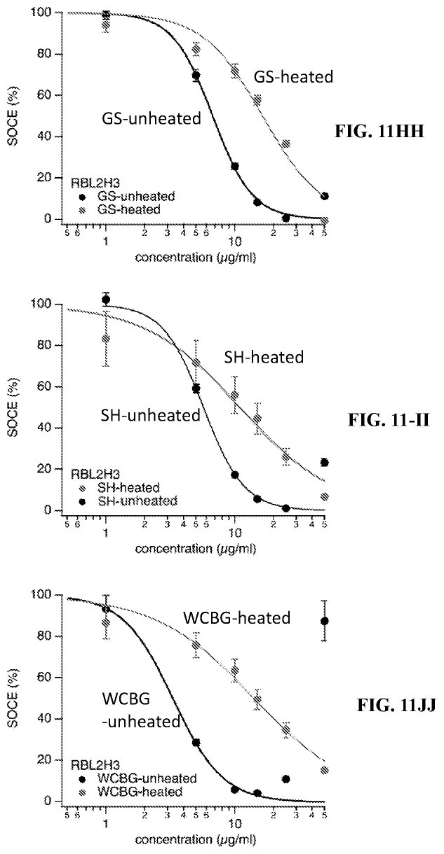

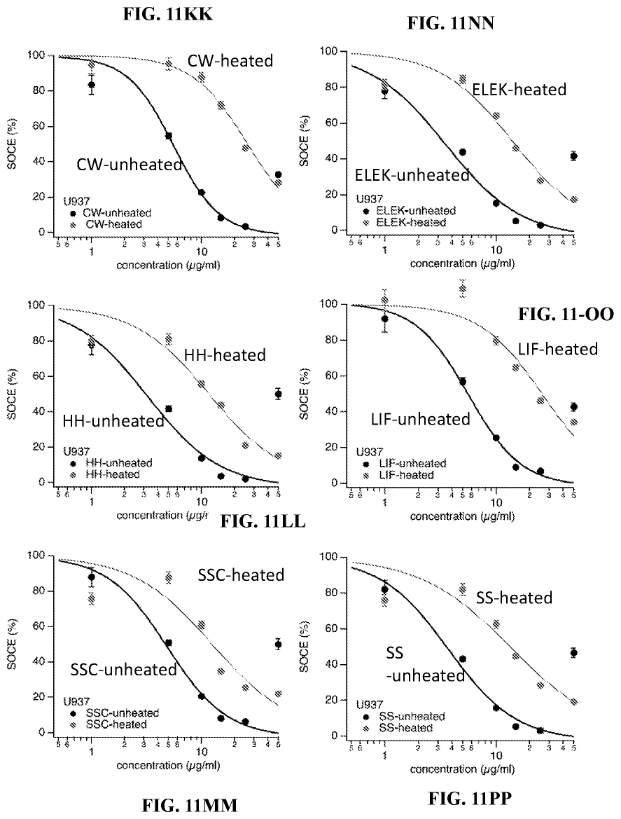

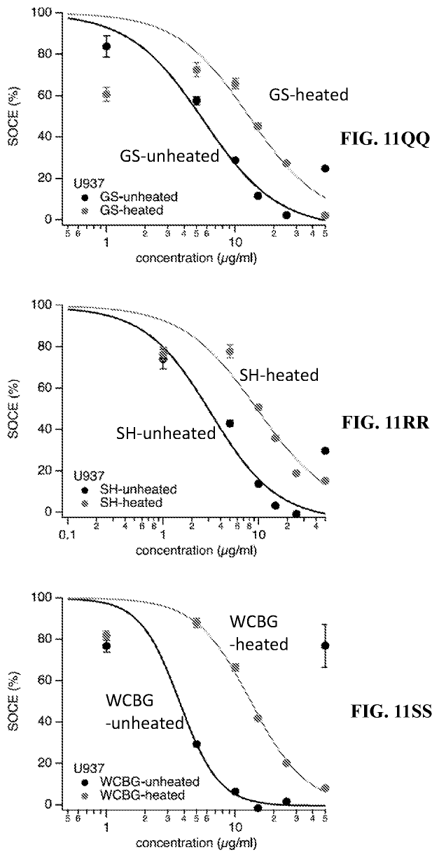

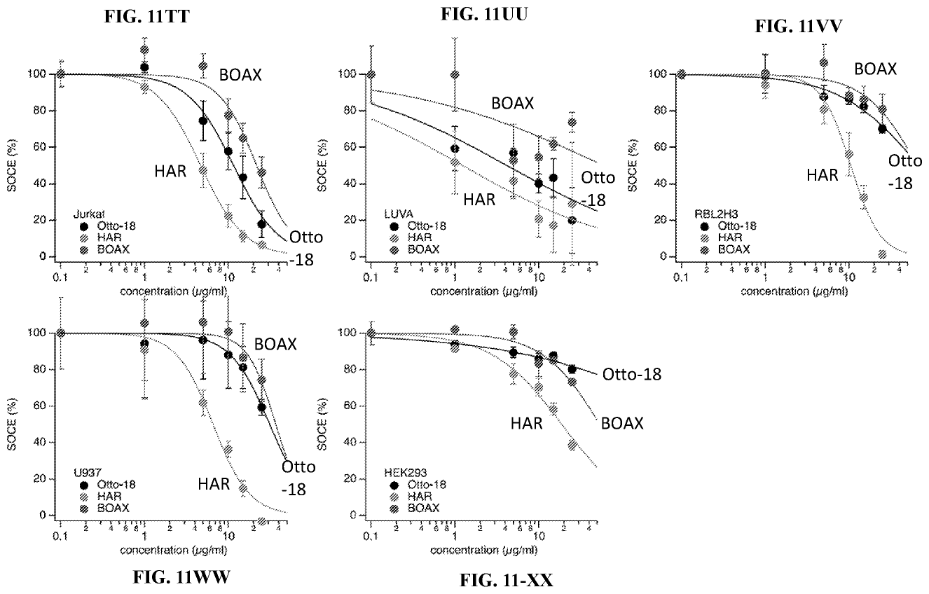

FIGS. 11A-11SS show dose-response curves for hemp varieties on store-operated calcium entry (SOCE) induced by thapsigargin in HEK-293 cells (FIGS. 11A-11-I), Jurkat cells (FIGS. 11J-11R), LUVA cells (FIGS. 11S-11AA), RBL-2H3 cells (FIGS. 11BB-11JJ), U937 (FIGS. 11KK-11SS). Briefly, SOCE inhibition experiments were performed as described in Example 8, using each of the hemp extracts listed below and in Tables 6A and 6B at six concentrations each in each of the five cell types listed above to obtain dose response curves, as shown in FIGS. 11A-11SS. The effects on SOCE amplitude were plotted in a dose-dependent manner to determine the IC50 for each tested extract. IC50 values are compiled in Table 6A and Table 6B for unheated and heated experiments, respectively. All data are averages of three independent runs±SEM. Hemp extracts were either extracted at room temperature (“unheated”, black data points) or exposed, after extraction, to an increased temperature of 115° C. for 60 min (“heated”, gray data points) and were tested at 6 different doses from 1 to 50 μg/ml. Hemp extracts used in FIGS. 11A-11SS are: Sour Space Candy (SSC), Hawaiian Haze (HH), Special Sauce (SS), Suver Haze (SH), White CBG (WCBG), Elektra (ELEK), Cherry Wine (CW), Lifter (LIF), Grape Soda (GS). Results from these experiments indicate that IC₅₀ of hemp extracts can be modulated by adjusting the temperature to which cannabinoids are exposed during extraction or thereafter. For example, these results indicate that increasing temperature can increase the IC₅₀ value of cannabinoids for inhibiting store-operated calcium entry in human cells. Furthermore, the data show that the tested extracts had the most potent effect on SOCE in Jurkat cells, when extracts were unheated (Table 6A). When heated, the tested extracts were most potent in Jurkat and Luva cells (Table 6B).

FIGS. 11TT-11-XX show dose-response curves for hemp varieties on store-operated calcium entry (SOCE) induced by thapsigargin in HEK-293, Jurkat, U937, LUVA, and RBL-2H3 cells. Hemp extracts (cold extracted and unheated) were tested at 6 different doses from 1 to 50 μg/ml. The effects on SOCE amplitude were plotted in a dose-dependent manner to determine the IC₅₀ for each tested compound (see Table 6A for determined IC₅₀ values). All data are averages of three independent runs±SEM. Tested hemp varieties were: Otto-18, Harlequin (HAR), and BOAX. Results showed that Harlequin had the lowest IC₅₀ values, indicating that Harlequin was the most potent variety of the three tested varieties.

Table 6A shows SOCE IC₅₀ values of various unheated hemp extracts tested on various cell lines.

| IC50 (μg/mL) | Jurkat | U937 | Luva | RBL-2H3 | HEK293 |
|---|---|---|---|---|---|
| Sour Space Candy (SSC) | 2.54 | 4.86 | 4.65 | 4.88 | 13.15 |
| Hawaiian Haze (HH) | 2 | 3.22 | 3.26 | 4 | 8.23 |
| Special Sauce (SS) | 2.52 | 3.72 | 6.85 | 3.96 | 8.86 |
| Suver Haze (SH) | 2.44 | 3.18 | 6.19 | 4.5 | 7.95 |
| White CBG (WCBG) | 1.4 | 3.7 | 4.5 | 3.31 | 7.86 |
| Elektra (ELEK) | 2.3 | 3.52 | 4.05 | 4.76 | 10.18 |
| Cherry Wine (CW) | 6.27 | 5.57 | 6.79 | 5.72 | 12.17 |
| Lifter (LIF) | 3.48 | 5.63 | 4.28 | 5.72 | 12.66 |
| Grape Soda (GS) | 4 | 5.67 | 7.49 | 6.74 | 16.51 |
| BOAX | 21.84 | 36.85 | 59.7 | 66 | 53.77 |
| HAR | 4.62 | 6.57 | 1.23 | 10.5 | 18.83 |
| Otto-18 | 11.54 | 31.17 | 4.18 | 68 | 1052.3 |

Table 6B shows SOCE IC₅₀ values of various heated hemp extracts tested on various cell lines.

| IC50 (μg/mL) | Jurkat | U937 | Luva | RBL-2H3 | HEK293 |
|---|---|---|---|---|---|
| Sour Space Candy (SSC) | 5.64 | 12.45 | 4.41 | 4.88 | 17.56 |
| Hawaiian Haze (HH) | 6.23 | 11.71 | 4.41 | 17.58 | 44.85 |
| Special Sauce (SS) | 6.66 | 13.3 | 8.18 | 14.49 | 47.03 |
| Suver Haze (SH) | 5.95 | 9.62 | 5.74 | 10.96 | 21.19 |
| White CBG (WCBG) | 7.61 | 13.2 | 7.85 | 14.16 | 19.76 |
| Elektra (ELEK) | 6.69 | 14.02 | 4.93 | 16.79 | 33.81 |
| Cherry Wine (CW) | 7.61 | 26.23 | 6.12 | 24.67 | 42.24 |
| Lifter (LIF) | 6.47 | 25.85 | 5.06 | 25.73 | 41.61 |
| Grape Soda (GS) | 9.45 | 12.77 | 8.12 | 16.51 | 39.52 |

### Example 12

### Effects on the TRPM7 Pathway

This example shows effects of treatment with cannabinoids, such as CBGA, on cell physiology, for example, as it pertains to the TRPM7 pathway. Receptor agonists stimulate receptors (R) and G proteins (G), resulting in activation of phospholipase C (PLC), which produces the second messenger inositol 1,4,5-trisphosphate (IP₃) and causes the release of Ca²⁺ from the endoplasmic reticulum (ER) through TP receptors (IP₃R). This store depletion of Ca²⁺ is sensed by STIM molecules in the (ER), which then couple to and open calcium release-activated calcium (CRAC) channels in the plasma membrane (PM). The ensuing store-operated calcium entry (SOCE) causes a long-lasting increase in intracellular calcium concentration that can cause production and release of inflammatory cytokines as well as cell proliferation (e.g. cancer and fibrosis). TRPM7 is a dual-function protein with both ion channel and kinase activities. It is found both in the plasma and ER membranes and participates in calcium signaling and SOCE in several ways. The ion channel function enables Ca²⁺ and Mg²⁺ influx and helps filling the ER store with Ca²⁺. The kinase function can phosphorylate targets that enhance GPCR signaling to promote Ca²⁺ release and store depletion as well STIM signaling to enable and promote SOCE. Therefore, blocking the kinase activity by, for example, treatment with CBGA would suppress STIM coupling to CRAC channels and indirectly reduce SOCE, in many cases.

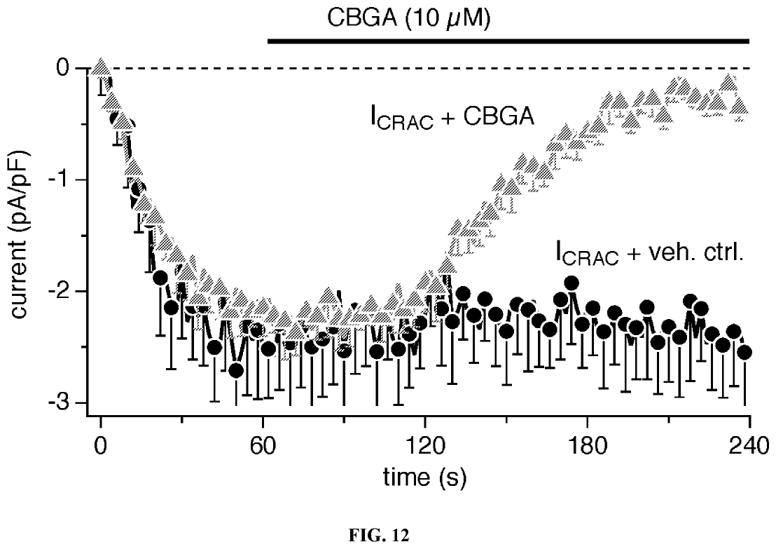

The ability of CBGA to inhibit store-operated calcium entry (SOCE) within cells was experimentally interrogated (see FIG. 12). Jurkat cells were perfused with 50 μM IP₃ to induce store depletion and activation of CRAC channels (the resulting Ca²⁺ inward current is known as I_{CRAC}). I_{CRAC} is a long-lasting inward current at −120 mV membrane potential (black trace) that can be blocked by 10 μM CBGA (gray trace). FIG. 12 shows treatment with 10 μM CBGA completely blocks the inward calcium current (gray trace), while treatment with a vehicle control (veh. ctrl.) does not affect IP₃-induced I_{CRAC} (black trace).

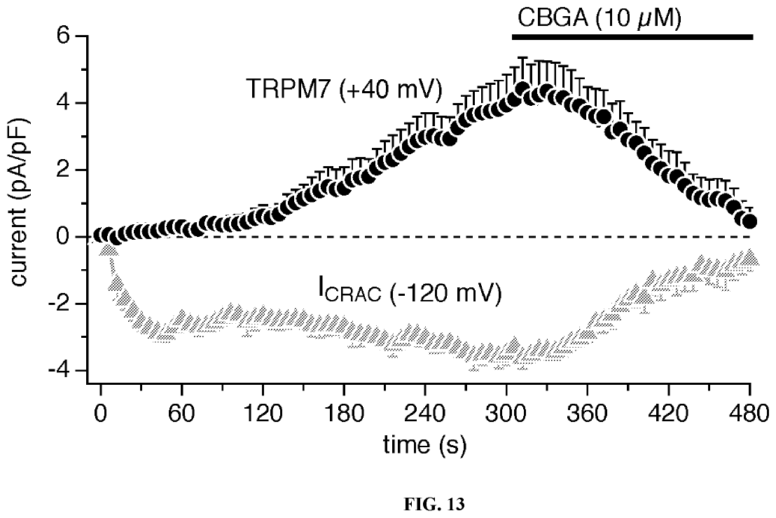

FIG. 13 shows an experiment using the same protocol and cell type (Jurkat) as in FIG. 12 over a longer period of time. Inward CRAC currents were induced at −120 mV (gray trace) and outward currents at +40 mV (black trace, where CRAC currents reverse and are essentially absent). At the +40 mV voltage potential, TRPM7 channels produce monovalent outward currents. Perfusion of 50 μM IP3 activates I_{CRAC} at −120 mV as above and the removal of intracellular ATP slowly activates TRPM7 current at +40 mV. Application of 10 μM CBGA blocks both outward TRPM7 and inward CRAC currents (see FIG. 13, black and gray traces, respectively). Without being bound by theory, CBGA may also block TRPM7's kinase activity, similar to other TRPM7 blockers such as NS8593.

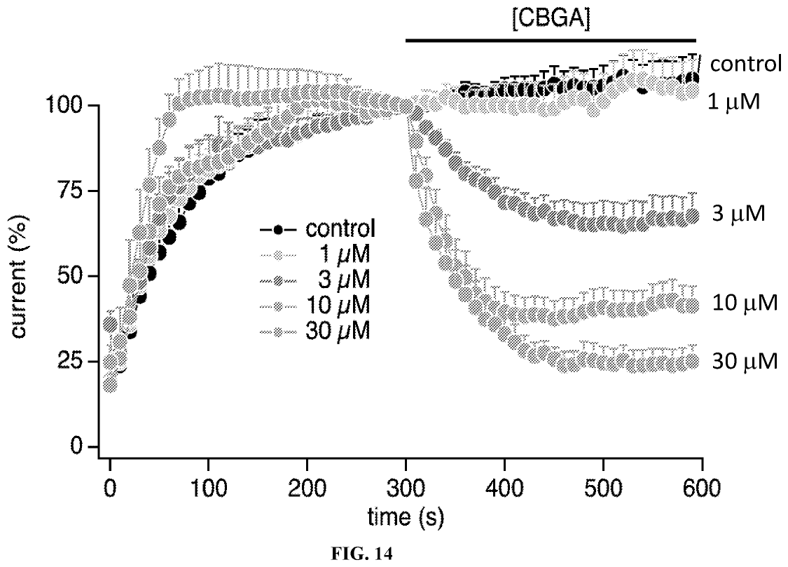

Activation of TRPM7 over-expressed in HEK293 cells by perfusing the cells with intracellular solution containing 0 ATP and 0 Mg²⁺, resulting in fast and maximal activation of TRPM7 outward currents at +40 mV is shown in FIG. 14. Application of 0 μM (control), 1 μM, 3 μM, 10 μM, or 30 μM CBGA causes dose-dependent block of TRPM7 current.

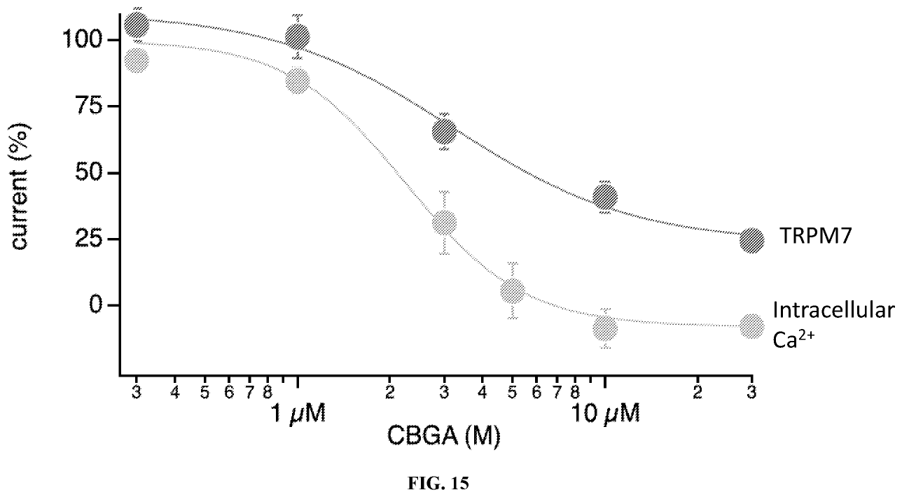

Data from dose-response curves for the inhibition of TRPM7 currents (dark gray symbols) obtained in experiments shown in FIG. 14 and SOCE-mediated increases in intracellular Ca²⁺ (light gray symbols) are shown in FIG. 15. Results from these experiments show that CBGA blocks both mechanisms with similar low micromolar IC₅₀ values of about 3 μM and 2 μM, respectively.

### Example 13

### CBGA Treatment of Chronic Kidney Disease

Chronic kidney disease (CKD) is amongst the 10 top leading causes of death in America. Among the main risk factors for CKD are diabetes and high blood pressure. TRPM7 belongs to the Transient Receptor Potential Melastatin family of ion channels. It is a Ca²⁺- and Mg²⁺-conducting ion channel fused with a functional kinase. TRPM7 can play a key role in a variety of diseases, including neuronal death in ischemia and fibrosis of the lung, liver and heart.

The Unilateral Ureteral Obstruction (UUO) model is a mouse model of kidney disease that is associated with and characterized by progressive tubulointerstitial injury and fibrosis. This model can be used to identify many of the molecular and cellular events that occur in progressive kidney fibrosis. In some cases, TRPM7 may be upregulated during inflammatory renal damage in this UUO mouse model, particularly in tubular epithelial cells. The TRPM7 inhibitor NS8593 can inhibit cell proliferation in a kidney cell-line model and ameliorates the progression of kidney damage and fibrosis in the UUO mouse model. TRPM7 represents a promising therapeutic target in kidney fibrosis and TRPM7 inhibitors may act as anti-fibrotic pharmacological tools.

C57BL/6 male mice (6 wks weighing 20-25 g) were used in the UUO model. Under isoflurane (3.0% for induction and 1.5% for maintenance) anesthesia, ureteral obstruction was achieved by ligating the left ureter with a 3-0 silk suture through a left lateral incision. Control and experimental groups for these experiments were created as follows:

Group 1: Control (5 mL/kg body weight) 5% ethanol and 5% Tween80 in 0.9% NaCl

Group 2: CBGA (10 mg/kg body weight) 2 mg/mL in 5% ethanol, 5% Tween80 and 0.9% NaCl

Group 3: CBD (10 mg/kg body weight) 2 mg/mL in 5% ethanol, 5% Tween80 and 0.9% NaCl

Group 4: CBGA (10 mg/kg body weight)+CBD (10 mg/kg body weight), 2 mg/mL each in 5% ethanol, 5% Tween80 and 0.9% NaCl

Mice were injected once daily with 10 mg/kg cannabinoids (CBGA, CBD, or both) as well as vehicle control daily beginning immediately after surgery and until Day 6. Mice were euthanized at Day 7 after surgery. Obstructed kidneys (UUO) and non-obstructed contralateral kidneys (CLK) were collected and stored.

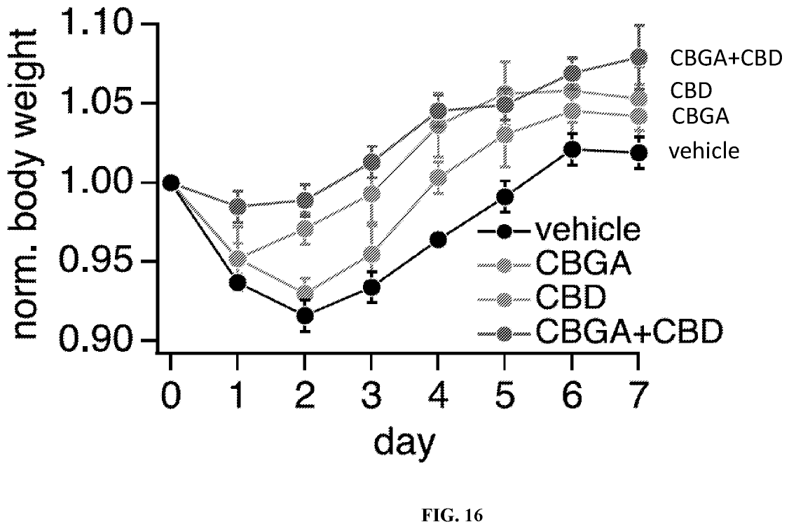

Body weight of mice was measured daily before injection (see FIG. 16 and Table 7). Body weight measurements were normalized by the weight before surgery, and the average is indicated in FIG. 16 (n=5). Black circles represent the vehicle treatment control group, light gray circles are data points from the CBGA treatment group, medium gray circles are from the CBD treatment group, and dark gray circles are data from the CBGA+CBD treatment group. It was observed that the CBGA-treated group of mice presented a faster recovery of body weight loss and less kidney atrophy compared to the group treated with vehicle.

TABLE 7
physiological parameters of UUO mice at day 7.

| | | kidney | kidney | | |
|---|---|---|---|---|---|
| | | weight | weight | water | |
| | body | (mg) | (mg) | intake | urine |
| | weight (g) | CLK | UUO | (mL) | (mL) |
| vehicle | 20.56 ± 0.20 | 171 ± 0.4 | 137 ± 0.8^{#} | 4.3 ± 0.2 | 1.24 ± 0.16 |
| CBGA | 21.55 ± 0.60 | 167 ± 0.7 | 163 ± 0.9 | 4.7 ± 0.2 | 1.88 ± 0.17 |
| CBD | 22.08 ± 0.45 | 175 ± 0.7 | 172 ± 0.8 | 4.9 ± 0.4 | 2.01 ± 0.44 |
| CBGA + | 22.85 ± 0.21** | 175 ± 0.3 | 167 ± 0.5 | 4.5 ± 0.4 | 1.54 ± 0.22 |
| CBD | | | | | |

Mice were sacrificed at day 7 after surgery and collected CLK and UUO kidneys (n = 5).
**p < 0.01 vs. vehicle. ^{#}p < 0.05 vs. CLK kidney.

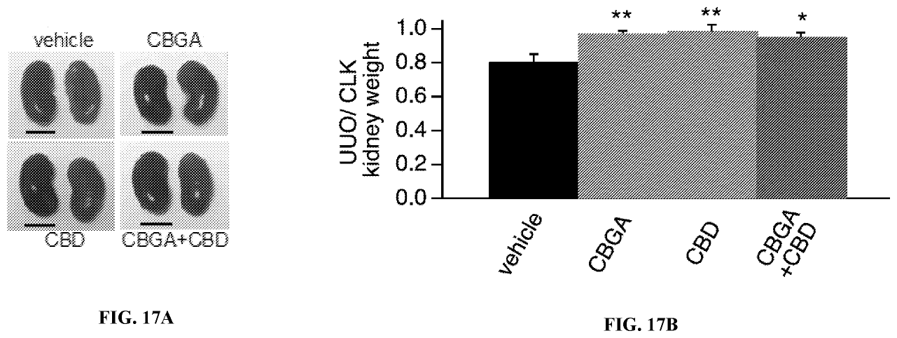

Images of representative CLK (left side of each panel) and UUO kidneys (right side of each panel) isolated from UUO mice were taken following euthanization at day 7 after ureteral obstruction surgery (FIG. 17A). Scale bars represent 5 mm. During experimentation, the vehicle control group was injected with vehicle daily (n=5, upper left panel). CBGA (10 mg/kg, n=5, upper right panel), CBD (10 mg/kg, n=5, lower left panel) and CBGA+CBD (each 10 mg/kg, n=5, lower right panel) were injected intraperitoneally daily from post-surgery day 0 to day 6. The weight of UUO kidney was measured at day 7 (see FIG. 17B and Table 7). Each UUO kidney weight was normalized to the weight of the corresponding CLK kidney. From left to right in FIG. 17B, bars represent: vehicle control group, CBGA treatment group, CBD treatment group, and CBGA+CBD treatment group. *p<0.05, **p<0.01 vs. vehicle UUO kidneys. The cis(+) vehicle control group lost kidney weight during experimentation and CBGA treatment of mice ameliorated the loss of kidney weight.

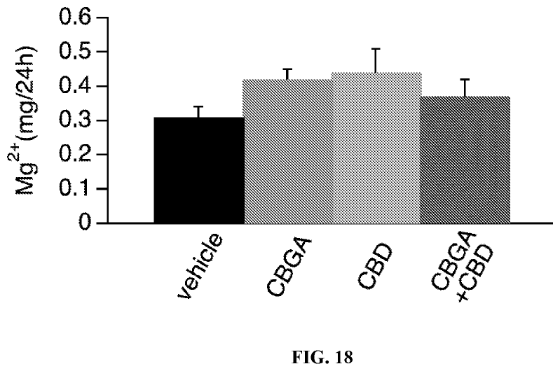

The level of magnesium was measured in urine collected from UUO mice at day 7 (see FIG. 18). Urine was collected using a metabolic cage for 24 hours before sacrifice. Magnesium concentration in urine was assessed by magnesium assay kit (n=5). Output of magnesium in urine was reduced in UUO mice treated with vehicle. Magnesium output in urine recovered in mice treated with cannabinoids (i.e., CBGA, CBD, or co-treatment with CBGA and CBD), relative to vehicle control treatment group results.

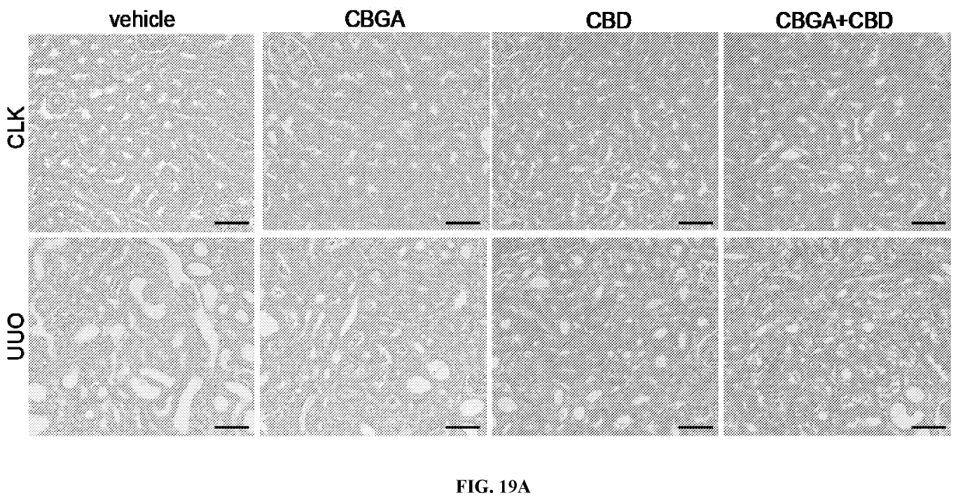

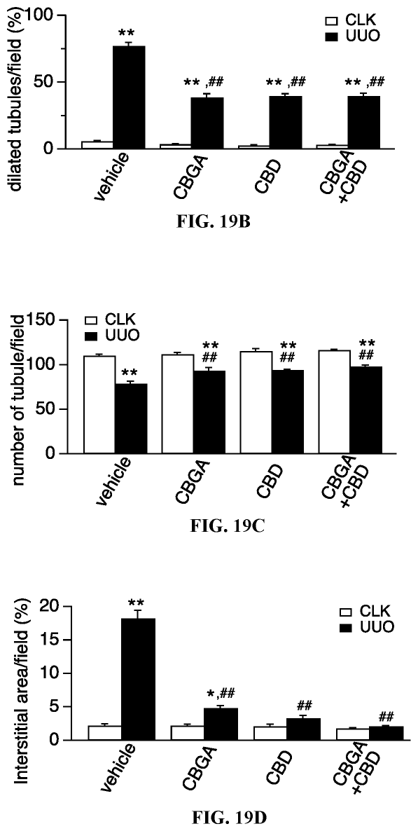

FIGS. 19A-19D depict the protection of kidney structure following cannabinoid treatment, as observed during experiments. Immunostaining was performed using Ki-67 as a cell proliferation marker, and ten randomly selected non-overlapping fields of sectioned and stained renal cortex samples from UUO and CLK kidneys were examined at 400× magnification in individual kidneys as follows: (3) quantification of the number of dilated and total renal tubules (e.g., as shown in FIGS. 19B and 19C, respectively); Interstitium areas were measured by image J using the slides of collagen type I immunostaining.

FIG. 19A shows representative images of H&E-stained CLK and UUO kidney sections (magnification ×200). Scale bars, 100 m. FIG. 19B shows a quantification of the number of dilated tubules observed on average in each field of view in histology sections. White bars represent CLK kidneys and black bars represent UUO kidneys. **p<0.01 vs. CLK kidneys, ##p<0.01 vs. vehicle UUO kidneys. As shown in FIG. 19B, CBGA inhibited tubular dilation in UUO kidneys relative to CLK kidneys. CBD and CBGA+CBD also inhibited tubular dilation in UUO kidneys, relative to CLK kidneys. FIG. 19C shows a quantification of the total number of renal tubules assessed per field of view in histology sections. **p<0.01 vs. CLK kidneys, ##p<0.01 vs. vehicle UUO kidneys. As shown in FIG. 19C, CBGA inhibited tubule loss in UUO kidneys, relative to CLK kidneys. CBD and CBGA+CBD also inhibited tubule loss in UUO kidneys, relative to CLK kidneys. FIG. 19D shows a quantification of interstitial area per field of view in histology sections. *p<0.05, **p<0.01 vs. CLK kidneys, ##p<0.01 vs. vehicle UUO kidneys. As shown in FIG. 19D, CBGA inhibited increases in interstitial area in UUO kidneys during experiments, relative to CLK kidneys and as compared to changes observed in vehicle-treated mice. CBD also inhibited interstitial area increase in UUO kidneys, relative to CLK kidneys and as compared to changes observed in vehicle-treated mice. CBGA+CBD had the strongest effect in inhibiting interstitial area increase in UUO kidneys, relative to CLK kidneys and as compared to changes observed in vehicle-treated mice. These results show that CBGA treatment suppressed the dilation of renal tubules and maintained the structure of renal tubules. In UUO kidneys treated with vehicle, the interstitial area increased due to substitution of extracellular matrix for collapsed renal tubules, which slightly increased in UUO kidneys treated with CBGA.

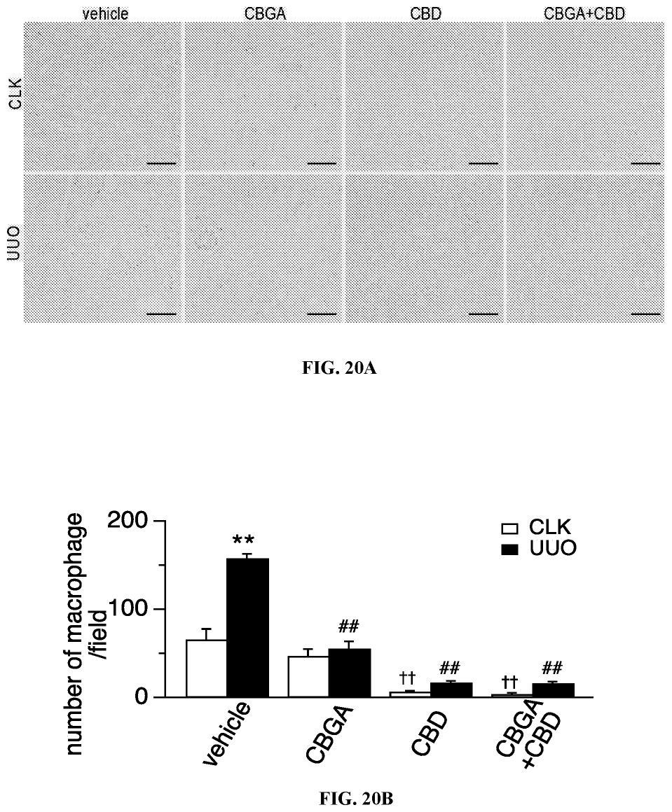

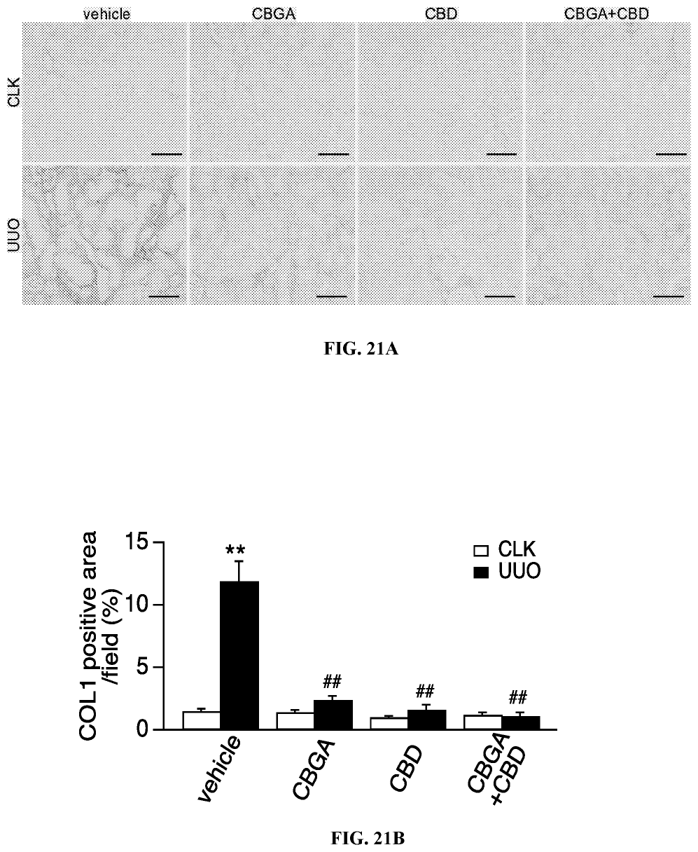

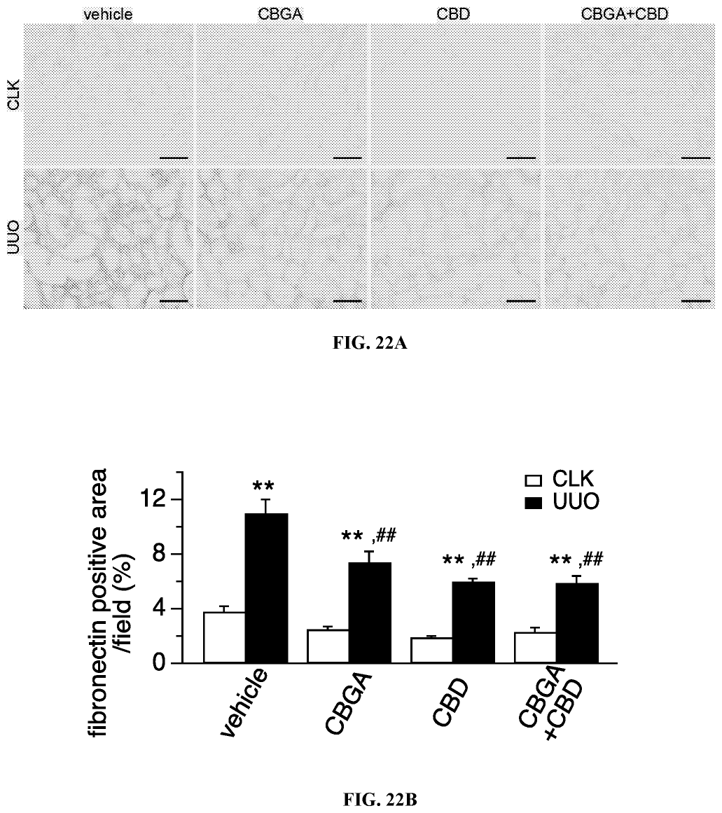

Immunohistochemical analysis. To evaluate renal fibrosis in UUO kidneys, immunoreactivities of F4/80 (macrophage marker), collagen type I, fibronectin, and α-smooth muscle actin (α-SMA) were determined using a standard biotin-streptavidin-peroxidase method. The staining-positive areas were calculated by Image J. The threshold was set to automatically compute the positive areas for each staining and the ratio of the positive areas to the whole interstitial area. Number of macrophages (FIG. 20A and FIG. 20B), collagen type I production in stained kidney tissue (FIG. 21A and FIG. 21B), and fibronectin production in stained kidney tissue (FIG. 22A and FIG. 22B) were quantified in UUO kidneys, showing that extracellular matrix production and/or deposition in kidney tissue were also affected with CBGA treatment.

FIG. 20A shows representative images of kidney sections immunostained for F4/80 as a marker for macrophages in CLK (upper panels) and UUO kidneys (lower panels, magnification ×200) after vehicle or cannabinoid treatment (i.e., CBGA, CBD, or CBGA co-administered with CBD). Scale bars represent 100 m.

FIG. 20B shows a quantification of the number of macrophages counted in immunostained CLK kidney sections (white bars) and UUO kidney sections (black bar). **p<0.01 vs. CLK kidneys, ##p<0.01 vs. vehicle UUO kidneys. ††p<0.01 vs. vehicle CLK kidneys. These results show that CBGA inhibited macrophage infiltration in UUO kidneys and that CBD and CBGA+CBD strongly inhibited macrophage infiltration in UUO kidneys (e.g., more strongly than CBGA treatment).

FIG. 21A shows representative images of kidney sections immunostained for collagen type I in CLK (upper panels) and UUO kidneys (lower panels, magnification ×200) after vehicle or cannabinoid treatment (i.e., CBGA, CBD, or CBGA co-administered with CBD). Scale bars represent 100 m.

FIG. 21B shows a quantification of the average percentage of the collagen type I-positive area in kidneys in CLK kidneys (white bars) and UUO kidneys (black bar). The staining intensity in the interstitium was computed using Image J software. **p<0.01 vs. CLK kidneys, ##p<0.01 vs. vehicle UUO kidneys. These data show that CBGA inhibited collagen type I produce in UUO kidneys relative to the vehicle control group. CBD and CBGA+CBD were also shown to inhibit collagen type I produce in UUO kidneys relative to the vehicle control group.

FIG. 22A shows representative images of kidney sections immunostained for fibronectin in CLK (top panels) and UUO kidneys (lower panels, magnification ×200) with or without cannabinoid treatment. Scale bars represent 100 m.

FIG. 22B shows a quantification of the average percentage of the fibronectin-positive area in kidneys in CLK (white bar) and UUO (black bar) kidneys. The staining intensity in the interstitium was computed using Image J software. **p<0.01 vs. CLK kidneys. ##p<0.01, vs. vehicle UUO kidneys. These data show that CBGA inhibited fibronectin production in UUO kidneys relative to vehicle controls. CBD and CBGA+CBD were also shown to inhibit fibronectin production in UUO kidneys relative to vehicle controls.

These data suggest that CBGA may have an inhibitory effect on the progress of kidney fibrosis and a reno-protective effect against kidney disease.

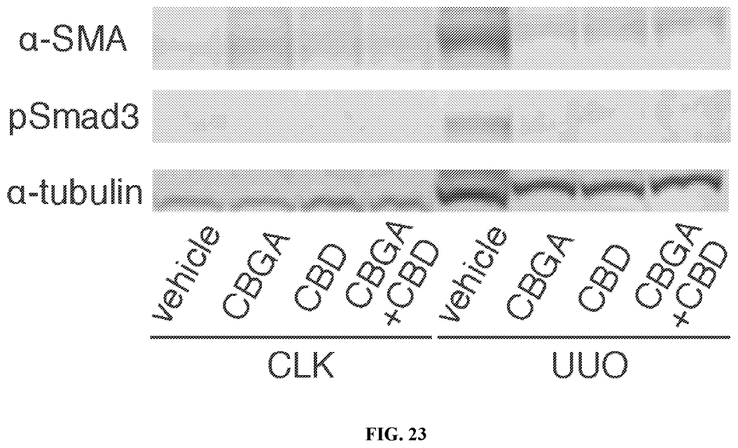

FIG. 23 shows representative images of Western blotting assay for α-SMA and phosphorylated Smad3 in UUO kidneys with vehicle or cannabinoid treatment. The protein expressions of α-SMA and phosphorylated Smad3 were examined in cortical kidney tissue using Western blotting, with α-tubulin used as an internal control. The data show that α-SMA and phosphorylated Smad3 proteins increased in UUO kidneys treated with vehicle and were suppressed in UUO kidneys treated with cannabinoids (e.g., CBGA, CBD, and co-treatment with CBGA and CBD).

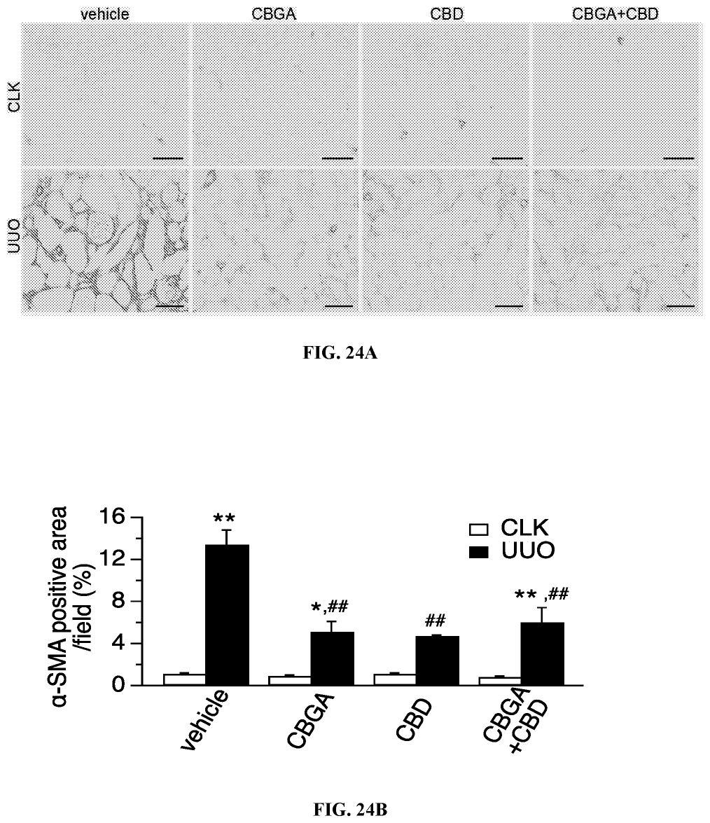

FIG. 24A shows representative images of kidney sections immunostained for α-SMA in CLK (top panels) and UUO kidneys (lower panels, magnification ×200) with or without cannabinoid treatment (e.g., CBGA, CBD, or co-treatment with CBGA and CBD). Scale bars represent 100 m.

FIG. 24B shows a quantification of the average percentage of the α-SMA-positive area in kidneys in CLK (white bar) and UUO (black bar) kidneys, as assessed in immunostained kidney sections. The staining intensity in the interstitium was computed using Image J software. *p<0.05, **p<0.01 vs. CLK kidneys. #p<0.05, ##p<0.01, vs. vehicle UUO kidneys. These data show that CBGA inhibited α-SMA production in UUO kidneys relative to vehicle control experiments. CBD and CBGA+CBD also inhibited α-SMA production in UUO kidneys relative to vehicle control experiments.

These experiments suggest that the cannabinoid Cannabigerolic Acid (CBGA), acting as a potent inhibitor of TRPM7 ion channel activity, may account for the anti-inflammatory and reno-protective in vivo effects in the UUO mouse model of progressive kidney fibrosis.

### Example 14

Cisplatin-induced Kidney Damage Model

This example shows assessment of effects of cannabinoid extracts in a cisplatin-induced kidney damage model.

Cisplatin is an anti-tumor drug that is used clinically in various malignancies. However, cisplatin is known to induce dose-related nephrotoxicity. Twenty to thirty percent of patients receiving cisplatin develop acute kidney damage. The cisplatin-induced mouse model is recognized as a reproducible model of acute kidney injury with a clinical relevance.

CBGA and/or CBD was injected intraperitoneally (i.p) 2 hours into C57BL/6 male mice (8 wks weighing 23-30 g) before cisplatin injection according to dosage schemes outlined below, and cisplatin-induced kidney damage was compared to normal kidneys. Sham treatment without cisplatin cis(−) was used as a negative control and cisplatin injection cis(+) without cannabinoid extract treatment was used as a positive vehicle control. Mice in group 1 (“vehicle”) received treatment with cisplatin (16 mg/kg body weight, lmg/mL in 0.90% NaCl, i.p) following intraperitoneal injection of vehicle control solution (5 mL/kg body weight) comprising 5% ethanol and 5% Tween80 in 0.9% NaCl. Mice in group 2 (“CBGA”) received treatment with cisplatin (16 mg/kg body weight, 1 mg/mL in 0.9% NaCl, i.p) following injection of CBGA (10 mg/kg body weight) 2 mg/mL in 5% ethanol, 5% Tween80 and 0.9% NaCl Mice in group 3 (“CBD”) received treatment with cisplatin (16 mg/kg body weight, 1 mg/mL in 0.9% NaCl, i.p) following intraperitoneal injection of CBD (10 mg/kg body weight) 2 mg/mL in 5% ethanol, 5% Tween80 and 0.9% NaCl. Mice in group 4 (“CBGA+CBD”) received treatment with cisplatin (16 mg/kg body weight, 1 mg/mL in 0.9% NaCl, i.p) following intraperitoneal injection of CBGA (10 mg/kg body weight)+CBD (10 mg/kg body weight) 2 mg/mL each in 5% ethanol, 5% Tween80 and 0.9% NaCl. Mice in group 5 (“cis(−)”) received sham injections comprising 0.9% NaCl (16 mL/kg body weight) following intraperitoneal injection of vehicle control solution (5 mL/kg body weight) 5% ethanol, 5% Tween80 and 0.9% NaCl.

Mice were euthanized at Day 3 following cisplatin injection, and kidneys were harvested and stored for processing and analysis.

FIG. 25 shows body weight measurements of mice was measured daily before vehicle or cannabinoids injection. Daily body weight measurements were normalized to Day 0 initial body weight (e.g., just prior to cisplatin administration), and the average is indicated (n=4-5). The body-weight loss in cisplatin-induced nephritis mice with vehicle treatment recovered with CBGA treatment. CBD and CBGA+CBD didn't affect body-weight loss in mice with cisplatin-induced nephritis.

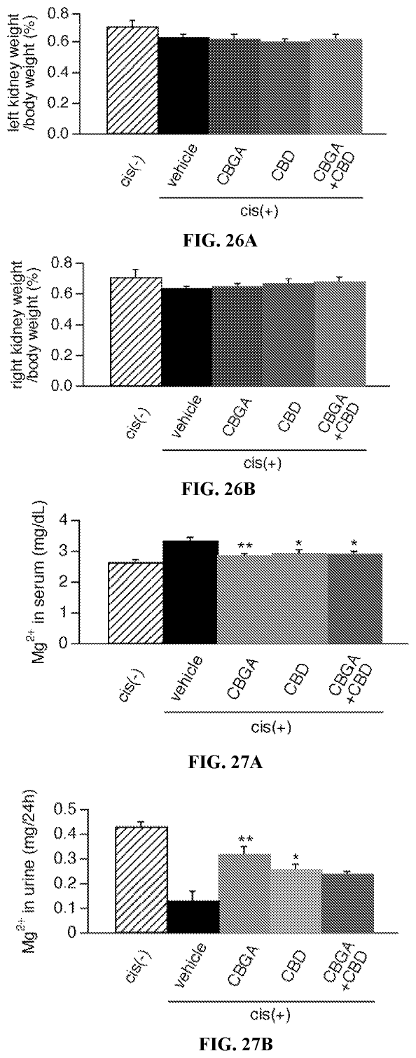

FIG. 26. The weight of kidney from cisplatin injected mice at day 3. Each left (FIG. 26A) and right (FIG. 26B) kidney weight were normalized to the body weight of each mouse. From left to right, bars represent: sham treatment group, vehicle control group, CBGA treatment group, CBD treatment group, and CBGA+CBD treatment group. The kidney weight was reduced in cisplatin-induced nephritis mice with vehicle treatment. No significant difference was determined for CBGA, CBD or CBGA+CBD treatment. Numerical values for body weight, kidney weight, water intake, and urine collected measurements can be found in Table 8.

TABLE 8
Measured physiological parameters of cisplatin-induced mice at day 3.

| | | kidney weight | kidney weight | water intake | |
|---|---|---|---|---|---|
| | body weight (g) | (mg) left | (mg) right | (mL) | urine (mL) |
| cisplatin(−) | 24.59 ± 1.03 | 172 ± 14.3 | 175 ± 16.7 | 4.65 ± 0.62 | 1.586 ± 0.137 |
| vehicle | 18.36 ± 0.58** | 116 ± 1.9** | 117 ± 6.2** | 1.14 ± 0.13** | 0.513 ± 0.198** |
| CBGA | 20.85 ± 1.29 | 128 ± 8.2** | 139 ± 10.8 | 2.50 ± 0.50* | 1.162 ± 0.128 |
| CBD | 20.23 ± 0.82* | 121 ± 5.0** | 132 ± 6.8 | 1.79 ± 0.37** | 1.042 ± 0.131 |
| CBGA + CBD | 19.70 ± 0.66* | 123 ± 7.3** | 135 ± 8.1 | 1.14 ± 0.16** | 0.877 ± 0.073** |

Mice were sacrificed at day 3 after cisplatin administration and collected kidneys (n = 4-5); *p < 0.05; **p < 0.01 vs. cisplatin(−).

FIGS. 27A-27B. Levels of magnesium at day 3 in mice administrated cisplatin are shown in FIGS. 27A-27B. Blood was collected when mice were sacrificed and then serum was separated from blood. Urine was collected using a metabolic cage for 24 hours before sacrifice. Magnesium concentration in urine was assessed by magnesium assay kit (n=4-5). *p<0.05 vs. cisplatin (+) vehicle treatment group. An increase in serum magnesium concentration (FIG. 27A) and a reduction in output of magnesium in urine (FIG. 27B) were observed in cisplatin-induced nephrotic. Without being bound by theory, the data suggest that these results are a result of loss of kidney function. CBGA maintained magnesium regulation in cisplatin-induced nephritis mice, for example, as demonstrated by increased blood serum concentration of magnesium (FIG. 27A) and reduction in magnesium loss in the urine (FIG. 27B) (e.g., relative to “vehicle” group, which was treated with cisplatin and cannabinoid delivery vehicle solution). CBD and CBGA+CBD protected magnesium regulation in cisplatin-induced nephritis mice as well as CBGA, e.g., relative to “vehicle” cisplatin-treated group.

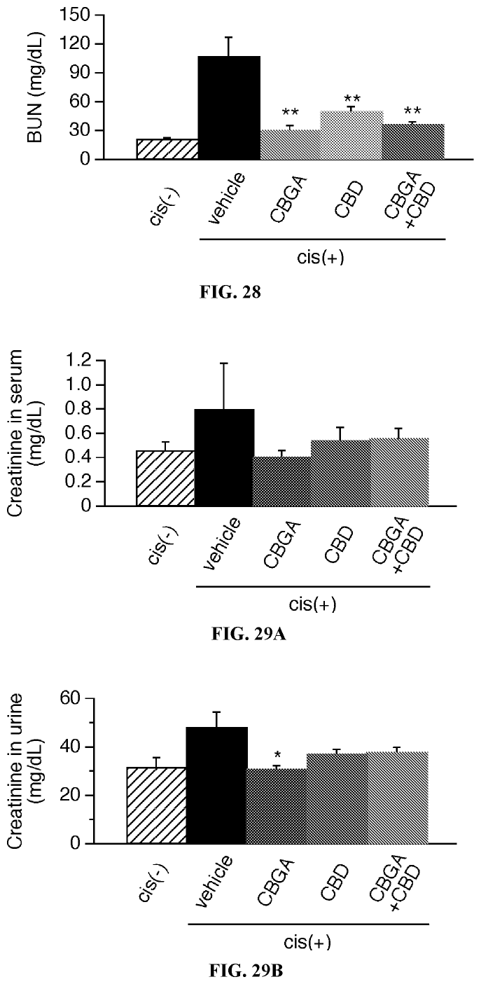

Blood and urine were collected and analyzed to check kidney function. Blood was collected following euthanization, and urine was collected for 24 hrs before euthanasia in metabolic cages. Markers of kidney function such as creatinine and BUN (Blood Urea Nitrogen) levels did not change between vehicle group and CBGA or CBD treatment groups in the cisplatin model. Creatinine levels and BUN or urea value were measured from blood serum and urine samples to evaluate the kidney function. FIG. 28 shows measured BUN levels in cisplatin-induced nephrotic mice at day 3BUN in serum were assessed by assay kit (n=4-5). *p<0.05, **p<0.01 vs. cis(+) vehicle treatment group. CBGA maintained kidney function in cisplatin-induced nephritis mice experiments and did not increase blood urea nitrogen (BUN) (FIG. 28). CBD and CBGA+CBD also maintained kidney function in cisplatin-induced nephritis mice as determined by BUN concentration levels. FIG. 29A-29B show measured creatinine levels in cisplatin-induced nephrotic mice at day 3. Urine was collected using a metabolic cage for 24 hours before sacrifice. Creatinine was assessed by an assay kit (n=4-5). Creatinine concentration in serum (FIG. 29A) and creatinine in urine (FIG. 29B) were maintained in cannabinoid-treated mice. *p<0.05 vs. cis(+) vehicle treatment group.

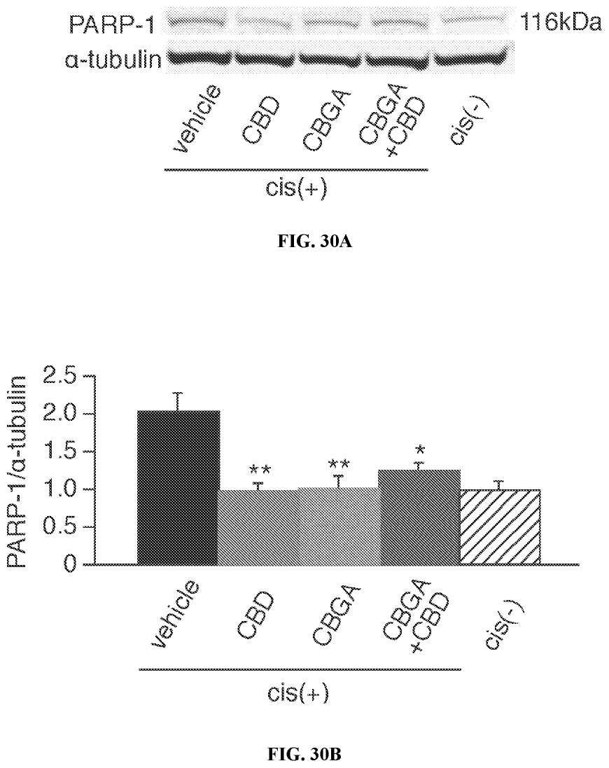

PARP-1 in cisplatin- and cannabinoid-treated kidney samples was evaluated by Western blot quantification. PARP activity increases to repair DNA damage, while it induces apoptosis when the cells have severe damage and can not be repaired in cisplatin-induced inflammatory kidneys. FIG. 30A shows a representative PARP-1 (full-length, 116 kDa) Western blot in sham-treated or cisplatin-induced nephritic kidneys with vehicle or cannabinoid treatments. (FIG. 30A) PARP-1 in kidney tissue was evaluated by quantification of Western blot results, wherein α-tubulin was used as an internal control to normalize PARP-1 band quantification. Cisplatin increased the prevalence of PARP-1, indicating that cisplatin induced kidney cell damage and led to apoptosis in cisplatin-treated mice. PARP-1 was reduced in mice treated with cisplatin and cannabinoids, relative to mice treated with cisplatin and “vehicle” solution (FIG. 30B). Densitometric analysis was performed from the result of western blotting and presented as a bar graph (n=4-5). From left to right, FIG. 30B shows “vehicle” control group data (e.g., cisplatin and vehicle solution treatment), CBGA treatment group, CBD treatment group, and CBGA+CBD treatment group. The rightmost bar represents the sham treatment (“cis(−)”), wherein mice received vehicle injections instead of both cisplatin and cannabinoid extracts as a negative control. **p<0.01 vs. cis(+) vehicle treatment group. CBGA reduced the amount of PARP-1 protein detected in cisplatin-induced nephritis mice. CBD and CBGA+CBD also reduced the amount of PARP-1 in cisplatin-induced nephritis mice.

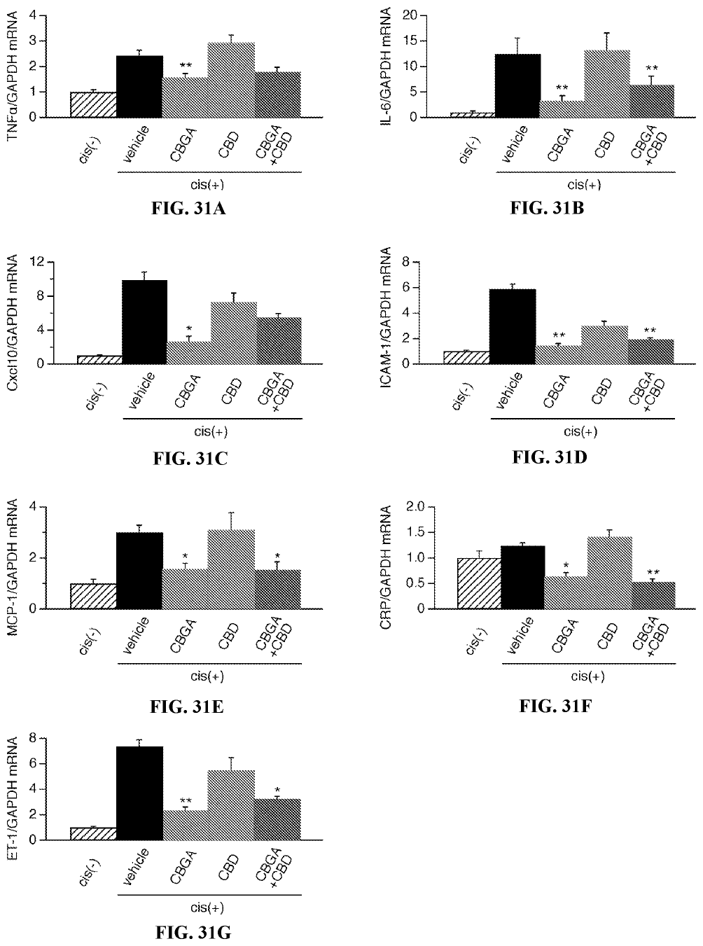

FIGS. 31A-31G show mRNA expression analysis of several cytokines and inflammation markers indicative of nephritic damage markers used to evaluate kidney injury in cisplatin mouse model experiments. Alleviation of kidney damage by drug injection was evaluated using qRT-PCR analysis of expression of the following cytokines and nephritic damage markers: TNF-α, IL-6, Cxcl10, MCP-1, ICAM1, CRP and endothelin-1 (ET-1). qRT-PCR data was normalized using measured GAPDH internal control values. From left to right in each of FIGS. 31A-31G, sham treatment (“cis(−)”), “vehicle” control group (e.g., treated with cisplatin and cannabinoid vehicle), CBGA treatment group (e.g., treated with cisplatin and CBGA), CBD treatment group (e.g., treated with cisplatin and CBD), and CBGA+CBD treatment group (e.g., treated with cisplatin, CBGA, and CBD) are shown. *p<0.05, **p<0.01 vs. cis(−) sham treatment group. The mRNA expressions of (FIG. 31A) tumor necrosis factor alpha (TNF-α), (FIG. 31B) interleukin-6 (IL-6), (FIG. 31C) C-X-C motif chemokine ligand 10 (CxCl 10), (FIG. 31D) intercellular adhesion molecule-1 (ICAM-1), (FIG. 31E) monocyte chemoattractant protein-1 (MCP-1), (FIG. 31F) C-reactive protein (CRP, a marker of acute kidney injury) and (FIG. 31G) endothelin-1 (ET-1) were measured. In all cases, consistent with increased inflammation, cis(+)/vehicle was significantly increased over cis(−), with the exception of FIG. 31F. In all cases, CBGA significantly reduced the cis(+)-induced increase of the inflammatory markers. Statistical analysis was performed using a one-way ANOVA and a post hoc analysis using Bonferroni/Dunn analysis.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A pharmaceutical composition comprising cannabigerolic acid (CBGA) and a second cannabinoid compound, wherein the CBGA is present in an amount from 1 mg to 2500 mg, and the second cannabinoid compound is present in an amount from 1 mg to 2500 mg.

2. The pharmaceutical composition of claim 1, wherein the CBGA is present in an amount from 5 mg to 1200 mg.

3. The pharmaceutical composition of claim 1, wherein the second cannabinoid compound is present in an amount from 5 mg to 1200 mg.

4. The pharmaceutical composition of claim 1, wherein the pharmaceutical composition inhibits secretion of inflammatory cytokines from at least one immune cell.

5. The pharmaceutical composition of claim 4, wherein at least one immune cell type is a lymphocyte.

6. The pharmaceutical composition of claim 4, wherein at least one immune cell type is a monocyte or a macrophage.

7. The pharmaceutical composition of claim 4, wherein at least one immune cell type is a microglia cell.

8. The pharmaceutical composition of claim 1, wherein the composition inhibits secretion of inflammatory cytokines by at least two immune cell types.

9. The pharmaceutical composition of claim 8, wherein at least one immune cell type is a lymphocyte and at least one immune cell type is a monocyte or a macrophage.

10. The pharmaceutical composition of claim 8, wherein at least one immune cell type is a lymphocyte and at least one immune cell type is a mast cell.

11. (canceled)

12. (canceled)

13. The pharmaceutical composition of claim 1, wherein the second cannabinoid and the CBGA have an additive, supra-additive, or sub-additive effect as measured by combination indices (CI) according to the method of isoboles.

14. The pharmaceutical composition of claim 1, wherein the second cannabinoid is cannabidiolic acid (CBDA), cannabidivarin (CBDV), cannabigerol (CBG), cannabidiol (CBD), tetrahydrocannabinolic acid (THCA), cannabigerovarinic acid (CBGVA), or tetrahydrocannabivarinic acid (THCVA).

15-27. (canceled)

28. The pharmaceutical composition of claim 1, wherein the pharmaceutical composition is in a unit dose form.

29-31. (canceled)

32. A method of treating a pain, inflammation, or fibrosis in a subject in need thereof, comprising administering to the subject a pharmaceutical composition comprising cannabigerolic acid (CBGA) and a second cannabinoid compound, wherein the CBGA is present in an amount from 1 mg to 2500 mg, and the second cannabinoid compound is present in an amount from 1 mg to 2500 mg.

33. The method of claim 32, wherein the CBGA is present in an amount from 5 mg to 1200 mg.

34. The method of claim 32, wherein the second cannabinoid compound is present in an amount from 5 mg to 1200 mg.

35-44. (canceled)

45. The method of claim 32, wherein the second cannabinoid is cannabidiolic acid (CBDA), cannabidivarin (CBDV), cannabigerol (CBG), cannabidiol (CBD), tetrahydrocannabinolic acid (THCA), cannabigerovarinic acid (CBGVA), or tetrahydrocannabivarinic acid (THCVA).

46-91. (canceled)

92. A method of treating fibrosis in a subject in need thereof, comprising administering to the subject a pharmaceutical composition comprising a therapeutically effective amount of cannabigerolic acid (CBGA).

93. The method of claim 92, wherein the therapeutically effective amount of CBGA is between 0.1-50 mg/kg.

94-100. (canceled)

101. The method of claim 92, wherein the fibrosis is associated with Chronic Kidney Disease (CKD).

102-122. (canceled)
